(19) United States
(12) Patent Application Publication
(10) Pub. No.: US 2026/0151492 A1
(43) Pub. Date: Jun. 4, 2026

(54) DENDRIMER CONJUGATES OF ANTIDEPRESSANT AND ANTIPSYCHOTIC AGENTS AND THEIR METHODS OF USE

(71) Applicant: THE JOHNS HOPKINS UNIVERSITY, Baltimore, MD (US)

(72) Inventors: Kannan RANGARAMANUJAM, Baltimore, MD (US); Sujatha KANNAN, Baltimore, MD (US); Kunal PARIKH, Baltimore, MD (US); Anjali SHARMA, Baltimore, MD (US); Wathsala LIYANAGE, Baltimore, MD (US)

(21) Appl. No.: 19/106,541

(22) PCT Filed: Aug. 25, 2023
(86) PCT No.: PCT/US2023/072932

§ 371 Date: Feb. 25, 2025

Related U.S. Application Data
(60) Provisional application No. 63/401,483, filed on Aug. 26, 2022.

(51) Int. Cl. A61K 47/59; A61K 47/61; A61P 25/24; A61P 25/28

(52) U.S. Cl. CPC A61K 47/595; A61K 47/61; A61P 25/24; A61P 25/28

(57) ABSTRACT

Formulations of dendrimer conjugated to one or more antidepressant or antipsychotic agents, having greater selectivity of targeting to cell types and decreased risk of side effects, and methods of use thereof, have been developed. Preferably, the dendrimer-conjugated antidepressant or antipsychotic agents selectively bind to one or more receptors on the surface or inside the target cells. The formulations are suitable for enteral or parenteral delivery for treating one or more diseases, conditions, and injuries in the central and peripheral nervous system.

### CROSS-REFERENCE TO RELATED APPLICATIONS

This application claims benefit of U.S. Provisional Application No. 63/401,483 filed Aug. 26, 2022, entitled DENDRIMER CONJUGATES OF ANTIDEPRESSANT AND ANTIPSYCHOTIC AGENTS AND THEIR METHODS OF USE′ by The Johns Hopkins University, listing inventors Kunal Parikh, Kannan Rangaramanujam, Sujatha Kannan, and Anjali Sharma, hereby incorporated herein by reference in its entirety.

### FIELD OF THE INVENTION

The invention is generally in the field of antipsychotic/antidepressant drug formulations, specifically dendrimer-antipsychotic/antidepressant conjugates for selective delivery to the nervous system and peripheral sites of disease, and to specific receptors on target cells.

### STATEMENT REGARDING FEDERALLY SPONSORED RESEARCH

None.

### BACKGROUND OF THE INVENTION

Depression is a chronic and recurring illness that may require lifelong treatment with different modalities. A significant proportion of patients with depression remain inadequately treated, especially in primary care settings. Nonadherence and premature discontinuation of treatment are important factors that may significantly contribute to suboptimal outcomes. Adverse effects associated with the use of antidepressant drugs (ADs) are some of the most common factors responsible for nonadherence and the discontinuation of treatment. Studies have shown that up to 43% of patients with depression may discontinue antidepressants due to treatment-emergent adverse effects. Existing frontline treatments have significant side effects, are not effective in up to 33% of patients, and can take up to six weeks to achieve clinical efficacy in patients who do respond.

The introduction of tricyclic antidepressants (TCAs) and monoamine oxidase inhibitors in the 1950s revolutionized the treatment of depression. Since then, the search for more selective and possibly better tolerated ADs has continued. Rational drug development led to selective serotonin reuptake inhibitors (SSRIs). SSRIs have become the first-line drugs for the treatment of depression among several other indications. Following the marketing success of SSRIs, many newer generation antidepressants have gained approval as treatments for depression, including serotonin and noradrenaline reuptake inhibitors (e.g., venlafaxine, desvenlafaxine and duloxetine), bupropion (a noradrenaline and dopamine reuptake Inhibitor), mirtazapine (noradrenaline and selective serotonin antagonist) and trazodone (serotonin antagonist and reuptake inhibitor). With the exception of agomelatine (melatonin receptor agonist with 5-HT_{2C} receptor antagonist properties), all other agents primarily act through the modulation of monoaminergic neurotransmission. More recently, the US Food and Drugs Administration (FDA) has approved four additional antidepressants for the treatment of depression, vilazodone, levomilnacipran, vortioxetine, and combination dextromethorphan/bupropion.

Over the years, there has been a consistent effort to develop more efficacious ADs with better safety and tolerability profiles. Tricyclic antidepressants (TCAs) increase levels of norepinephrine and serotonin, two neurotransmitters, and block the action of acetylcholine, another neurotransmitter. These may restore the balance in these neurotransmitters in the brain to alleviate depression. In addition to relieving depression, tricyclic antidepressants also cause sedation and somewhat block effects of histamine.

There is no unequivocal evidence to support clinically significant differences in efficacy and tolerability among the various newer antidepressant agents and controversies remain. No clinically significant differences were found in the efficacy of SSRIs and TCAs. Differences in tolerability between TCAs and SSRIs appear to be modest. In addition, concerns about safety and tolerability related to the long-term use of newer generation antidepressants have been raised.

The Centre for Adverse Reactions Monitoring (CARM) continues to receive reports of sexual dysfunction associated with the use of antidepressants and antipsychotics. Since 1965, the most frequently reported medicines have been fluoxetine, citalopram, paroxetine, venlafaxine, risperidone and clozapine.

Disorders of sexual functioning can be classified into four categories:

Sexual desire disorders, including partial or total lack of libido.

Sexual arousal disorders including erectile dysfunction and lack of vaginal lubrication.

Orgasm disorders, including premature, delayed or absent orgasm (anorgasmia); also failure of ejaculation.

Sexual pain disorders including dyspareunia and vaginismus.

The prevalence of sexual dysfunction in major depression may be up to 70% of patients. Most often this takes the form of lack of libido. Sexual desire tends to improve with treatment of depression; however, successful antidepressant treatment often causes other detrimental effects on sexual function. Antidepressant-associated sexual dysfunction may also result from inadequate treatment of depression, comorbid alcohol abuse, comorbid physical illness, relationship problems, or a combination of these factors.

Management of sexual dysfunction during treatment with antidepressants can be difficult, in part due to often multifactorial etiology. Options for patients experiencing problems, include reducing dose (may increase risk of relapse), drug holidays (may jeopardize compliance with treatment; does not allow for spontaneity; withdrawal symptoms may be experienced in patients using antidepressants with short half-lives), switching antidepressant (may increase risk of relapse or different side effects); continuing treatment (‘wait and see’), or addition of an reversal agent (e.g., phosphodiesterase inhibitor which may cause additional side effects.

Newer generation antidepressant drugs (ADs) are widely used as the first line of treatment for major depressive disorders and are considered to be safer than tricyclic agents. Several side effects are transient and may disappear after a few weeks following treatment initiation, but potentially serious adverse events may persist or ensue later. These encompass gastrointestinal symptoms (nausea, diarrhea, gastric bleeding, dyspepsia), hepatotoxicity, weight gain and metabolic abnormalities, cardiovascular disturbances (heart rate, QT interval prolongation, hypertension, orthostatic hypotension), genitourinary symptoms (urinary retention, incontinence), sexual dysfunction, hyponatremia, osteoporosis and risk of fractures, bleeding, central nervous system disturbances (lowering of seizure threshold, extrapyramidal side effects, cognitive disturbances), sweating, sleep disturbances, affective disturbances (apathy, switches, paradoxical effects), ophthalmic manifestations (glaucoma, cataract) and hyperprolactinemia. At times, such adverse events may persist after drug discontinuation, yielding iatrogenic comorbidity. Other areas of concern involve suicidality, safety in overdose, discontinuation syndromes, risks during pregnancy and breast feeding, as well as risk of malignancies. Thus, the rational selection of ADs should consider the potential benefits and risks, likelihood of responsiveness to the treatment option and vulnerability to adverse events. The findings of this review should alert the physician to carefully review the appropriateness of AD prescription on an individual basis and to consider alternative treatments if available.

A significant proportion of patients generally do not achieve complete response and remission. Antipsychotics are commonly used as adjunctive therapy in adults. To date, the US Food and Drug Administration has approved four atypical antipsychotics (aripiprazole, quetiapine, brexpiprazole, and olanzapine) for this purpose. However, augmentation with antipsychotics is associated with a higher discontinuation rate and more adverse events (AEs) than antidepressant monotherapy. Adjunctive antipsychotics with antidepressants are often associated with movement disorders and seizures compared with antidepressant monotherapy in children and adolescents with depression.

Individuals with attention deficit disorders, and variations thereof including those diagnosed with attention deficit disorder as well as those having symptoms thereof, (“ADHD”) are another class of individuals for whom current treatments are of limited efficacy and have numerous side effects. ADHD drugs include methylphenidate, amphetamine, atomoxetine, clonidine, guanfacine, viloxazine, and their analogs/modifications. These drugs are commonly used in patients with depression, PTSD, and bipolar disorder. See https://www.webmd.com/add-adhd/adhd-medication-chart and https://www.additudemag.com/adhd-medication-for-adults-and-children.

Some of them have similar mechanisms of action to antidepressants/antipsychotics such as atomoxetine (SNRI), viloxazine (SNRI) and methylphenidate which blocks reuptake of dopamine and norepinephrine. Examples of antidepressants currently used to treat the symptoms of ADHD include bupropion, desipramine, imipramine, and nortriptyline.

ADHD drugs are generally divided into stimulant and non-stimulants. Both are generally associated with significant side effects associated with systemic, non-targeted delivery. Although non-stimulants typically are not as effective as stimulants, they are used in up to 30% of patients who cannot tolerate or do not benefit from stimulants. There is significant need for alternatives with reduced side effects and increased selectivity.

It is therefore an object of the present invention to provide formulations and methods of use thereof to increase the specificity of delivery with respect to specific cells and receptors, decrease the side effects of anti-depressants and anti-psychotics, and improve treatment efficacy (including onset of action).

### SUMMARY OF THE INVENTION

Formulations of dendrimer conjugated to one or more antidepressant or antipsychotic agents, having greater selectivity of targeting to cell types and decreased risk of side effects, and methods of use thereof, have been developed. Preferably, the dendrimer-conjugated antidepressant or antipsychotic agents selectively bind to one or more receptors on the surface or inside the target cells. The formulations are suitable for enteral or parenteral delivery for treating one or more diseases, conditions, and injuries in the central and peripheral nervous system.

Representative antidepressant and antipsychotic agents include Selective Serotonin Reuptake Inhibitors (SSRIs), Serotonin and Norepinephrine Reuptake Inhibitors (SNRIs), Norepinephrine-Dopamine Reuptake Inhibitors (NDRIs), Tricyclic Antidepressants (TCAs), Monoamine Oxidase Inhibitors (MAOIs), Benzodiazepines, GABA modulators (e.g., neurosteroids), antipsychotics, atypical antipsychotics, or analogues thereof. Preferred dendrimers include glucose dendrimers and PAMAM dendrimers, which may be modified through hydroxylation of functional groups, PEGylation, or other means to alter uptake. Preferred glucose dendrimers include G1, G2, and G3 glucose dendrimers, while preferred PAMAM dendrimers include G3, G4, G5, and G6 hydroxyl-terminated PAMAM dendrimers. In preferred embodiments, the antidepressant or antipsychotic agents are conjugated to the dendrimers by linkers, preferably cleavable by hydrolysis. In some forms, the linkers contain a triazole moiety.

Formulations can be administered orally, to a mucosal surface, or by injection.

Formulations are useful for the prevention, treatment, or management of the symptoms of disorders such as major depressive disorder, treatment-resistant depression, and post-partum depression, post-traumatic stress disorder, panic disorder, social anxiety disorder, anorexia nervosa, suicidal ideation, obsessive-compulsive disorder, premenstrual dysphoric disorder, anorexia, substance abuse disorders, epilepsy, bi-polar disorder, autism spectrum disorders, attention-deficit hyperactivity disorders, schizophrenia, cluster headaches, migraines, seizures, fibromyalgia, narcolepsy, obesity, Alzheimer's disease, Tourette's syndrome, pain such as neuropathic pain and chronic pain, phobias, and cardiovascular diseases.

### BRIEF DESCRIPTION OF THE DRAWINGS

FIGS. 1A and 1B are schematics showing an exemplary synthesis route for dendrimer-fluoxetine conjugate with a non-cleavable linkage using click chemistry. Fluoxetine is first conjugated to a linker with an azide moiety (FIG. 1A), followed by conjugation to a dendrimer modified with surface alkyne groups via azide-alkyne click reactions (FIG. 1B).

FIGS. 2A and 2B are schematics showing an exemplary synthesis route for dendrimer-paroxetine conjugate with a non-cleavable linkage. Paroxetine is first conjugated to a linker with an azide moiety (FIG. 2A), followed by conjugation to a dendrimer modified with surface alkyne groups via azide-alkyne click reactions (FIG. 2B).

FIGS. 3A and 3B are schematics showing an exemplary synthesis route for dendrimer-venlafaxine conjugate with an enzyme-cleavable ester linkage. Venlafaxine is first conjugated to a linker with an azide moiety (FIG. 3A), followed by conjugation to a dendrimer modified with surface alkyne groups via azide-alkyne click reactions (FIG. 3B).

FIGS. 4A and 4B are schematics showing an exemplary synthesis route for dendrimer-venlafaxine analog conjugate with a non-cleavable amide linkage. Venlafaxine analog is first conjugated to a linker with an azide moiety (FIG. 4A), followed by conjugation to a dendrimer modified with surface alkyne groups via azide-alkyne click reactions (FIG. 4B).

FIGS. 5A and 5B are schematics showing an exemplary synthesis route for dendrimer-citalopram analog conjugate with a non-cleavable amide linkage. Citalopram analog is first conjugated to a linker with an azide moiety (FIG. 5A), followed by conjugation to a dendrimer modified with surface alkyne groups via azide-alkyne click reactions (FIG. 5B).

FIG. 6 is a schematic showing a synthesis route for glucose dendrimer-fluoxetine conjugate with a non-cleavable linkage.

FIG. 7 is a schematic showing a synthesis route for the synthesis of glucose dendrimer-paroxetine conjugate with a non-cleavable linkage.

FIG. 8 is a schematic showing a synthesis route for the synthesis of glucose dendrimer-venlafaxine conjugate with an enzyme-cleavable ester linkage.

FIG. 9 is a schematic showing a synthesis route for the synthesis of glucose dendrimer-venlafaxine analog conjugate with a non-cleavable amide linkage.

FIG. 10 is a schematic showing a synthesis route for the synthesis of glucose dendrimer-citalopram analog conjugate with a non-cleavable amide linkage.

FIG. 11 is a schematic of the stepwise synthetic route for the synthesis of glucose dendrimer-DMT analog conjugate with a non-cleavable amino-alkyl linkage.

FIG. 12 is a schematic of the stepwise synthetic route for the synthesis of glucose dendrimer-lysergic acid diethylamide (LSD) conjugate with a non-cleavable amino-alkyl linkage.

FIG. 13 is a schematic overview of the main pharmacological targets of LSD, psilocybin, DMT, MDMA, and ketamine, the signaling cascades involved, hormonal modulation, as well as main behavioral outcomes following their administration in both animals and humans.

FIG. 14A is a schematic of the synthesis of a PAMAM dendrimer-norketamine conjugate. FIG. 14B is a schematic of the synthesis of a Glucose dendrimer-norketamine conjugate.

FIG. 15A is a graph of an NMDAR 1A/2B antagonist assay for glucose dendrimer-ketamine (IC50=4.54 μM), hydroxyl dendrimer-ketamine (IC50>100), and norketamine (IC50=6.96 μM). FIG. 15B is the % binding efficacy of the log concentration of compound in micromolar in a D2L human dopamine GPCR cell based agonist cAMP assay. Norketamine (solid circle), glucose dendrimer-ketamine EC50=13.08 micromolar (open circle), and hydroxyl dendrimer-ketamine EC50=4.263 micromolar (triangle). FIG. 15C is the % efficacy of the log concentration of ketamine in micromolar in the TA1 human trace amine GPCR cell based agonist cAMP assay. Norketamine (solid circle), glucose dendrimer-ketamine EC50=13.08 micromolar (open circle), and hydroxyl dendrimer-ketamine EC50-4.263 micromolar (triangle).

FIGS. 16A and 16B are graphs of wild type, knock out saline (controls) versus knockout mice treated with dendrimer-ketamine conjuate composite neurobehavior score of (FIG. 16A) and probability of survival over post natal day (FIG. 16B). FIG. 16C is a graph of the distance traveled (m); FIG. 16D is a graph of the speed at which the mice traveled; FIG. 16E is a graph of the time spent in corners.

FIGS. 17A and 17B are exemplary chemical structures of a dendrimer conjugated to D-Fluoxetine. FIG. 17C is an exemplary chemical structure of a dendrimer conjugated to D-Paroxetine. FIGS. 17D-17F are exemplary chemical structures of a dendrimer conjugated to D-Venlafaxine (FIGS. 17D and 17F) or a D-Venlafaxine analog (FIG. 17E). FIGS. 17G and 17H are exemplary chemical structures of a dendrimer conjugated to D-Citalopram (FIG. 17G) or a D-Citalopram analog (FIG. 17H).

### DETAILED DESCRIPTION OF THE INVENTION

### I. Definitions

The term “antidepressant agents” refer to compounds that modulate the reuptake of one or more monoamines (such as serotonin, noradrenaline, norepinephrine, dopamine) into the presynaptic cell. In some forms, “antidepressant agents” inhibit the reuptake of monoamines (such as serotonin, noradrenaline, norepinephrine, and dopamine) into the presynaptic cell; persistence of these monoamines in the synaptic cleft results in increased postsynaptic receptor stimulation and hence in increased postsynaptic transmission. In some forms, these effects, presumably, corrects or compensates for the physiological deficits that may underlie depression. Examples of drugs that inhibit monoamine reuptake are the tricyclic antidepressants (TCA), the selective serotonin reuptake inhibitors (SSRIs), the serotonin-norepinephrine reuptake inhibitors (SNRIs) and others. In some forms, “antidepressant agents” refers to compounds that inhibit the breakdown of monoamines (such as serotonin, noradrenaline, and dopamine) in the storage vesicles of the presynaptic cell. Preservation of these monoamines presumably improves the efficiency of synaptic transmission. This may correct or compensate for the physiological deficits that underlie depression. Drugs that inhibit monoamine breakdown include the monoamine oxidase inhibitors (MAOIs). In other forms, “antidepressant agents” refer to compounds that increase the reuptake of serotonin (tianeptine), increase the release of serotonin and/or norepinephrine (mirtazapine), act directly on serotonin and melatonin receptors (agomelatine) or otherwise influence synaptic neurotransmission. These drugs, including ADHD drugs such as methylphenidate, amphetamine, atomoxetine, clonidine, guanfacine, viloxazine, as well as gabapentin and lithium/lithium salts, are conjugated or complexed with dendrimer for selective delivery to neurons and activated microglia for prevention, treatment, or management of a variety of disorders. In some forms, “antidepressant agents” may also improve mental health or neurological disorders via anti-inflammatory effects.

The term “antipsychotic agent” refers to psychotropic compounds administered to manage one or more symptoms of a psychosis (e.g., including delusions, hallucinations, paranoia, or disordered thought), for example, in psychosis, schizophrenia and bipolar disorders, but also in a range of other disorders such as symptoms associated with a mood disorder, an anxiety disorder, or a non-neurological disorder. Antipsychotic agents inhibit dopaminergic or dopaminergic and serotonin transmission, and may also exert noradrenergic, cholinergic, and/or histaminergic blocking action.

The terms “active agent” or “biologically active agent” are used interchangeably to refer to a chemical or biological compound that induces a desired pharmacological and/or physiological effect, which may be prophylactic, therapeutic, or diagnostic. These may be a nucleic acid, a nucleic acid analog, a small molecule having a molecular weight less than 2 kD, more typically less than 1 kD, a peptidomimetic, a protein or peptide, carbohydrate or sugar, lipid, or a combination thereof. The terms also encompass pharmaceutically acceptable, pharmacologically active derivatives of agents, including, but not limited to, salts, esters, amides, prodrugs, active metabolites, and analogs. The term “therapeutic agent” refers to an agent that can be administered to treat one or more symptoms of a disease or disorder. The term “diagnostic agent” generally refers to an agent that can be administered to reveal, pinpoint, and define the localization of a pathological process. The diagnostic agents can label target cells that allow subsequent detection or imaging of these labeled target cells.

“Analog” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered an analog of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified compound, if it induces the same pharmacological effect, physiological effect, or both, as the specified compound. “Analog” can also refer to a modification including, but not limited to, hydrolysis, reduction, or oxidation products, of the compounds. Hydrolysis, reduction, and oxidation reactions are known in the art.

The term “therapeutically effective amount” refers to an amount of the therapeutic agent that, when incorporated into and/or onto dendrimers, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases.

The terms “inhibit” or “reduce” in the context of inhibition, mean to reduce, or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, dendrimer compositions including one or more inhibitors may inhibit or reduce the activity and/or quantity of diseased neurons by about 10%, 20%, 30%, 40%, 50%, 75%, 85%, 90%, 95%, or 99% from the activity and/or quantity of the same cells in equivalent tissues of subjects that did not receive, or were not treated with the dendrimer compositions. In some embodiments, the inhibition and reduction are compared at levels of mRNAs, proteins, cells, tissues, and organs. For example, an inhibition and reduction in the rate of neural loss, in the rate of decrease of brain weight, or in the rate of decrease of hippocampal volume, as compared to an untreated control subject.

The term “treating” or “preventing” mean to ameliorate, reduce or otherwise stop a disease, disorder or condition from occurring or progressing in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with depression are mitigated or eliminated, including, but are not limited to, reducing the level of anxiety, agitation, or restlessness, improving feelings of sadness, tearfulness, emptiness or hopelessness, increasing the quality of life of those suffering from the disease, decreasing the dose of other medications required to treat the disease, delaying the progression of the disease.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “biodegradable” generally refers to a material that will degrade or erode under physiologic conditions to smaller units or chemical species that are capable of being metabolized, eliminated, or excreted in vivo. The degradation time is a function of composition and morphology.

The term “dendrimer” includes, but is not limited to, a molecular architecture with an interior core, interior layers, or “generations” of repeating units regularly attached to this initiator core, and an exterior surface of terminal groups attached to the outermost generation.

The term “functionalize” means to modify a compound or molecule in a manner that results in the attachment of a functional group or moiety. For example, a molecule may be functionalized by the introduction of a molecule that makes the molecule a strong nucleophile or strong electrophile.

The term “targeting moiety” refers to a moiety that localizes to or away from a specific location. The moiety may be, for example, a protein, nucleic acid, nucleic acid analog, carbohydrate, or small molecule. The location may be a tissue, a particular cell type, a subcellular compartment, or a molecule such as a receptor.

The term “prolonged residence time” refers to an increase in the time required for an agent to be cleared from a patient's body, or organ or tissue of that patient. In certain embodiments, “prolonged residence time” refers to an agent that is cleared with a half-life that is 10%, 20%, 50% or 75% longer than a standard of comparison such as a comparable agent without conjugation to a delivery vehicle such as a dendrimer. In certain embodiments, “prolonged residence time” refers to an agent that is cleared with a half-life of 2, 5, 10, 20, 50, 100, 200, 500, 1000, 2000, 5000, or 10000 times longer than a standard of comparison such as a comparable agent without a dendrimer that specifically target specific cell types.

The terms “incorporated” and “encapsulated” refer to incorporating, formulating, or otherwise including an agent into and/or onto a composition that allows for release, such as sustained release, of such agent in the desired application. The agent or other material can be incorporated into a dendrimer, by binding to one or more surface functional groups of such dendrimer (by covalent, ionic, or other binding interaction), by physical admixture, by enveloping the agent within the dendritic structure, and/or by encapsulating the agent inside the dendritic structure.

As used herein, central nervous system (“CNS”) includes the brain and spinal cord. As used herein, peripheral nervous system (“PNS”) refers to the nerves other than in the brain and spinal cord.

“Hydroxyl-terminated,” as relates to dendrimers, refers to dendrimers that have a hydroxyl group on their surface. These hydroxyl groups are not attached to the termini of the dendrimers via a sugar moiety (such as a saccharide moiety).

“Sugar-terminated,” as relates to dendrimers, refers to dendrimers that contain a sugar moiety (such as a saccharide moiety) on their surface and not in their core.

“Sugar-based,” as relates to dendrimers, refers to dendrimers that contain a sugar moiety (such as a saccharide moiety) in their core, or their core and on their surface.

### II. Compositions

Dendrimer-active agent conjugates suitable for delivering one or more antidepressants or antipsychotics to one or more target cells expressing receptors therefor, such as neural cells, glial cells and/or peripheral cells enabled by the dendrimer, and further to specific receptors on these cells dictated by the drug, have been developed. Generally, the antidepressants or antipsychotics bind to a receptor on the surface of the target cells and/or a receptor inside the target cells. Exemplary target cells include, but not limited to, brain cells such as microglia, astrocytes, and/or neurons, for example, those within the site of pathology in the brain or the CNS; cells in the peripheral nervous system, such as peripheral neurons and glia, and/or peripheral cells such as gastrointestinal cells, cardiovascular cells, and immune system cells. The microglia and/or astrocytes to which the antidepressants or antipsychotics are delivered may be activated or inactive microglia and/or astrocytes.

The antidepressants or antipsychotics of the dendrimer-active agent conjugate binds to a target receptor on the surface of the target cell or inside the target cell. In some embodiments, when antidepressants or antipsychotics bind to the target receptor, the agent remains conjugated to the dendrimer. In these embodiments, following binding, the agent may be released from the dendrimer or may remain conjugated to the dendrimer. In some embodiments, the antidepressants or antipsychotics are released from the dendrimer in close proximity to the target receptor and then bind to the target receptor on the target neural and/or glial cell.

### A. Dendrimers

Dendrimers are three-dimensional, hyperbranched, monodispersed, globular and polyvalent macromolecules including surface end groups (Tomalia, D. A., et al., Biochemical Society Transactions, 35, 61 (2007); and Sharma, A., et al., ACS Macro Letters, 3, 1079 (2014)).

The term “dendrimer” includes, but is not limited to, a molecular architecture with an interior core (“G0”) and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, the dendrimers herein have a diameter between about 1 nm and about 60 nm, more preferably between about 1 nm and about 50 nm, between about 1 nm and about 40 nm, between about 1 nm and about 30 nm, between about 1 nm and about 20 nm, between about 1 nm and about 10 nm, or between about 1 nm and about 5 nm. In some embodiments, the diameter is between about 1 nm to about 2 nm. In some embodiments, the dendrimers have a diameter effective to penetrate blood brain barrier (“BBB”) of less than 5 nm and are retained close to or within target neural and/or glial cells for delivery of the agents conjugated thereto. In some embodiments, the dendrimers have a diameter effective to penetrate the BBB and to be internalized into target neural and/or glial cells for delivery of the agents conjugated thereto, such as for example, neurons, oligodendrocytes, astrocytes, microglial, and neuroglial support cells. In some embodiments, the dendrimers have a diameter effective to penetrate a barrier interface, such as a blood nerve barrier (“BNB”), and to be internalized into neural and glial cells of the peripheral nervous system for delivery of the agents conjugated thereto such as for example, neurons, Schwann cells, satellite cells, and neuroglial support cells. These are typically greater than 5 nm in diameter. In some embodiments, the dendrimers have a diameter effective to be retained in the peripheral circulation for delivery of the agents conjugated thereto to target cells of the peripheral nervous system. In some embodiments, the dendrimers have a diameter effective to be retained in the peripheral circulation for delivery of the agents conjugated thereto to target cells of the gastrointestinal system, cardiovascular system, and/or immune system.

In some embodiments, dendrimers have a molecular weight between about 500 Daltons and about 100,000 Daltons inclusive, between about 500 Daltons and about 50,000 Daltons inclusive, or between about 1,000 Daltons and about 20,000 Daltons inclusive. Dendrimer sizes <30,000 Da are preferred for transport across the BBB, and sizes of >50,000 Da are preferred for confinement to the periphery.

In some embodiments, the dendrimers have a hypercore (e.g., dipentaerythritol) and one or more monosaccharide branching units. In some embodiments, the monosaccharide branching units are conjugated to the core or the prior layer of monomers via linkers such as polyethylene glycol chains. In preferred embodiments, the hypercore is dipentaerythritol and the monosaccharide branching unit is glucose-based branching unit such as shown in Structures II-IV. In the most preferred embodiment, the dendrimers are made entirely of glucose building blocks. PAMAM dendrimers modified by sugar may also work, but dendrimers made of sugars, especially glucose, are most preferred. Particularly preferred glucose dendrimers are G1 to G3 glucose dendrimers, such as G1, G2, and/or G3 glucose dendrimers.

Preferred dendrimers are glucose dendrimers, although other dendrimers can be used. Suitable dendrimers scaffolds include, but are not limited to, poly(amidoamine), also known as PAMAM, or STARBURST™ dendrimers; polypropylamine (POPAM), polyethylenimine, polylysine, polyester, iptycene, aliphatic poly(ether), aromatic polyether dendrimers, dendrimer of a sugar (e.g., glucose, galactose, mannose, fructose, etc.), and copolymers thereof, such as a copolymer of a sugar and an alkylene glycol (e.g., a dendrimer formed by glucose and ethylene glycol building blocks). The dendrimers can have a plurality of surface functional groups, such as carboxylic, amine, hydroxyl, and/or acetamide. The terms “surface functional groups” and “terminal groups” are used interchangeably herein. The preferred dendrimers have surface hydroxyl groups to insure selective uptake in neurons and in activated microglia. In some embodiments, one or more of these surface functional groups are further modified with other molecules, such as further modified with a sugar (e.g., glucose, galactose, mannose, fructose, etc.) and/or a polyalkylene glycol, for example, polyethylene glycol, and thus have sugar molecules and/or polyalkylene glycols as terminal moieties/molecules. Preferred PAMAM dendrimers include hydroxyl-terminated PAMAM dendrimers, particularly G3 to G6 hydroxyl-terminated PAMAM dendrimers, such as G3, G4, G5, and G6 hydroxyl-terminated PAMAM dendrimers.

Dendrimers can be any generation including, but not limited to, generation 1, generation 2, generation 3, generation 4, generation 5, generation 6, generation 7, generation 8, generation 9, or generation 10.

In some embodiments, the dendrimer-active agent conjugates can be confined to the peripheral circulation and specifically target a particular tissue region and/or cell type, such as peripheral neural and glial cells and/or gastrointestinal, cardiovascular and/or immune system cells, by using higher generation dendrimer (such as generation 4, 5, or 6 PAMAM dendrimer, or generation 2, 3, or higher glucose-based dendrimers). Additionally, or alternatively, the dendrimer-active agent conjugates can be confined to the peripheral circulation by appropriate functionalization of the dendrimer (such as PEGylation).

In some embodiments, the dendrimers can specifically target a particular tissue region and/or cell type of the central nervous system (CNS), the peripheral nervous system (PNS), and/or the periphery, such as neurons and glia of the CNS, neurons and/or glia of the PNS, and/or peripheral cells such as gastrointestinal cells, cardiovascular cells, and/or immune system cells by using dendrimers of a certain generation, such as PAMAM dendrimers and/or glucose dendrimers of generation 2 (G2), G3, G4, and G5.

The term “PAMAM dendrimer” refers to poly(amidoamine) dendrimer, which may contain different cores, with amidoamine building blocks, and can have carboxylic, amine, acetamide, and/or hydroxyl terminations of any generation. In some embodiment, the dendrimers are generation (“G”) 4, 5 or 6 dendrimers. In the most preferred embodiment, the dendrimers are made entirely of glucose building blocks. PAMAM dendrimers modified by sugar may also work, but dendrimers made of sugars, especially glucose, are most preferred.

### Monosaccharide-Based Dendrimers

In some embodiments, the branching units include monosaccharides. In some embodiments, the monosaccharide branching units are conjugated to the core or the prior layer of monomers via linkers such as polyethylene glycol chains. In some embodiments, the monosaccharide branching units are glucose-based branching units. In some embodiments, the branching units can include PEG and/or alkyl chain linkers between different dendrimer generations. For example, the glucose layers are connected via PEG linkers and triazole rings. In some embodiments, the branching units are the same for each generation of dendrimers generated from the core. Therefore, for example, the branching units are glucose-based branching units for generating generation 1 dendrimers, for generating generation 2 dendrimers, and for generating generation 3 dendrimers.

In some embodiments, the dendrimers have a hypercore (e.g., dipentaerythritol) and one or more monosaccharide branching units. In some embodiments, the hypercore is dipentaerythritol and the monosaccharide branching unit is glucose-based branching unit. In further embodiments, spacer molecules can also be alkyl (CH₂)ₙ-hydrocarbon-like units.

In some embodiments, dendrimers synthesized using glucose building blocks, with a surface made predominantly of glucose moieties, allow specific targeting in cells including injured neurons, ganglion cells, and other neuronal cells in the brain, the eye, and/or in peripheral nervous system. In some embodiments, the glucose-based dendrimer selectively targets or is enriched inside target neural and/or glial cells. In some embodiments, the glucose-based dendrimer selectively targets or enriched on the surface of target neural and/or glial cells. In some embodiments, the glucose-based dendrimer selectively targets or enriched inside target neuronal cells and on the surface of the target neural and/or glial cells. In some embodiments, the glucose-based dendrimer selectively targets or enriched inside and/or on the surface of injured, diseased, and/or hyperactive neurons and/or glial cells.

In some cases, the dendrimers include an effective number of sugar molecules and terminal groups, for example, glucose and/or hydroxyl groups, for targeting to one or more neurons and/or glia of the CNS, PNS, and/or the eye. The terminal hydroxyl groups of these dendrimers may be part of terminal glucose molecules or extra hydroxyl groups that are not part of the glucose molecules, or a combination thereof. In some embodiments, all the terminal hydroxyl groups are part of the terminal glucose molecules. In some embodiments, the number of sugar molecules on the termination of dendrimer is determined by the generation number.

In some embodiments, dendrimers are made of glucose and oligoethylene glycol building blocks. Exemplary glucose dendrimers are shown in Structures V and VII.

Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. For example, the glucose dendrimer is a generation 2 glucose-based dendrimer that has 24 glucose molecules at the periphery and 6 embedded glucose molecules in the backbone held together by PEG segments.

Dendrimer compositions that can selectively accumulate inside neurons, particularly in the nucleus of injured and/or hyperactive neurons, referred to as “glucose dendrimers” have been developed. These dendrimers can also accumulate at a high level inside activated microglia. However, compared to hydroxyl dendrimers which primarily accumulate in microglia, these dendrimers primarily go to neurons. Glucose dendrimers are described in U.S. Ser. No. 63/327,610 “Dendrimer Compositions for Targeted Delivery of Therapeutics to Neurons” by The Johns Hopkins University, inventors Kannan Rangaramanujam, Rishi Sharma, Anjali Sharma, Sujatha Kannan, Nirnath Sah, Mira Sachdeva, and Siva P. Kambhampati filed Apr. 5, 2022.

Glucose dendrimers include (a) a central core, (b) one or more branching units, wherein the branching units are monosaccharide glucose-based branching units, optionally with a linker conjugated thereto; and optionally (c) one or more therapeutic, prophylactic and/or diagnostic agents. Generally, the one or more branching units are conjugated to the central core, and the surface groups of the dendrimer are monosaccharide glucose molecules. In some embodiments, the central core is dipentaerythritol, or a hexa-propargylated derivative thereof. In some embodiments, the branching unit is conjugated to the central core via a linker such as a hydrocarbon or an oligoethylene glycol chain. In a preferred embodiment, the branching units are β-D-Glucopyranoside tetraethylene glycol azide having the following structure,

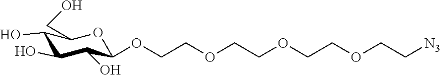

or peracetylated derivatives thereof.

In some embodiments, the glucose dendrimer is a generation 1, generation 2, generation 3, generation 4, generation 5, or generation 6 dendrimer. In one embodiment, the dendrimer is a generation 1 dendrimer having the following structure:

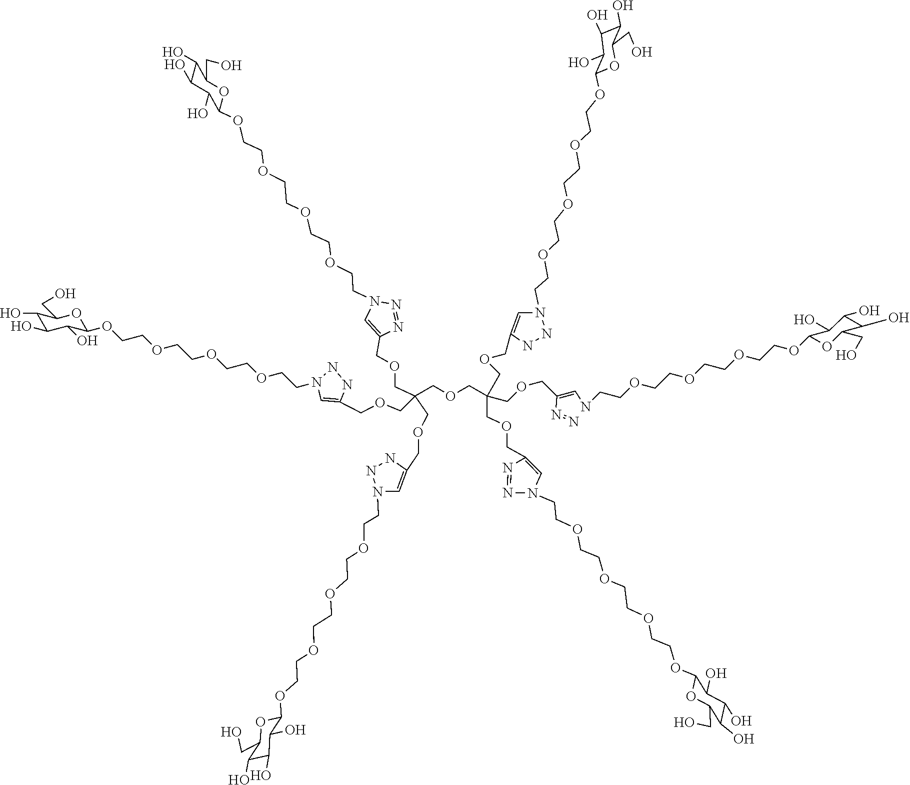

In a preferred embodiment, the dendrimer is a generation 2 dendrimer having the following structure:

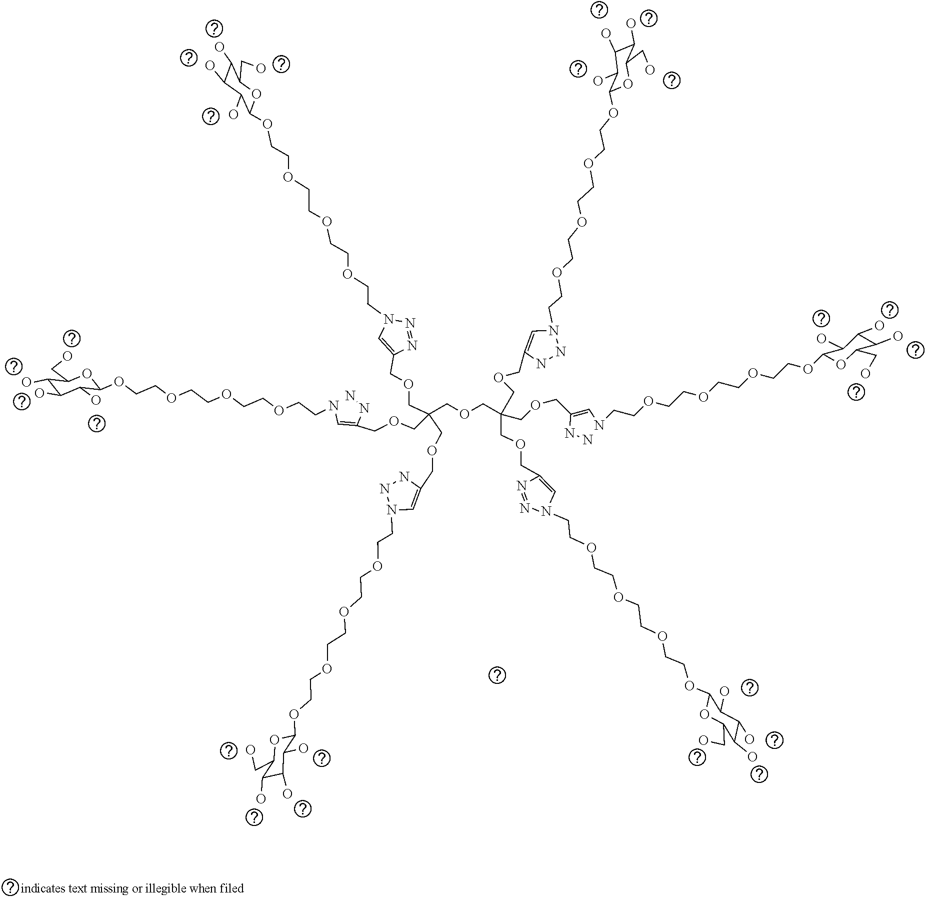

In some embodiments, the one or more therapeutic agents, prophylactic agents, and/or diagnostic agents are encapsulated, associated, and/or conjugated in the dendrimer, at a concentration of between about 0.01% to about 30%, preferably about 1% to about 20%, more preferably about 5% to about 20% by weight. In some embodiments, the dendrimer is conjugated to a small molecule, an antibody or antigen-binding fragment thereof, a nucleic acid, or a polypeptide. In some embodiments, the therapeutic agents conjugated to the dendrimer are anti-inflammatory agents, antioxidant agents, or immune-modulating agents. In other embodiments, the dendrimers are conjugated to one or more diagnostic agents such as fluorescent dyes, near infra-red dyes, SPECT imaging agents, PET imaging agents, and radioisotopes.

In some embodiments, the dendrimer and the therapeutic, prophylactic, or diagnostic agent(s) are conjugated via one or more linkers or coupling agents such as one or more hydrocarbon or oligoethylene glycol chains. Exemplary linkages are disulfide, ester, ether, thioester, and amide linkages.

### PAMAM Dendrimer

The term “PAMAM dendrimer” refers to poly(amidoamine) dendrimer, which may contain different cores, with amidoamine building blocks, and can have carboxylic, amine, acetamide, and/or hydroxyl terminations of any generation including, but not limited to, generation 1 to generation 10 PAMAM dendrimers. In some embodiment, the dendrimers are generation (“G”) 4, 5 or 6 dendrimers. In some embodiments, the PAMAM dendrimers have hydroxyl terminations or are surface modified with monosaccharides.

Generally, the complete architecture of dendrimers can be distinguished into the inner core moiety followed by radially attached branching units (i.e., generations) which are further decorated with chemical functional groups carrying desired terminal groups at the exterior surface of the dendrimers.

In some embodiments, the dendrimers are in nanoparticle form, as described in US 2011/0034422, US 2012/0003155, and US 2013/0136697. For example, the molecular weight of the dendrimers can be varied to prepare polymeric nanoparticles that form particles having properties, such as drug release rate, optimized for specific applications.

In some embodiments, different variations of dendrimers may be used as a delivery vehicle to conjugate and deliver one or more active agents, including, but not limited to, dendrons and tectodendrimers. Dendrons are dendritic wedges that comprise one type of functionality at the core (functional groups, f=1) and another at the periphery (f=8, 16, 32, etc. . . . ). Tectodendrimers are generally composed of a central dendrimer with multiple dendrimers attached at its periphery.

### 1. Core

In some embodiments, dendrimers are prepared using methods in which the dendrimer is assembled from a multifunctional core, which is extended outward by a series of reactions. A multifunctional core moiety allows stepwise addition of branching units (i.e., generations) around the core.

Exemplary chemical structures suitable as core moieties include dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′″-silanetetrayltetrakis (propane-1-thiol), 3,3-divinylpenta-1,4-diene, 3,3′,3″-nitrilotripropionic acid, 3,3′,3″-nitrilotris(N-(2-aminoethyl) propanamide), 3,3′,3″,3′-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl) silane, 1-Thioglycerol, 2,2,4,4,6,6-hexachloro-1,3,5,2l5,4l5,6l5-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl)benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2,2′,2″-nitrilotris(ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof. In some embodiments, the core moiety is chitosan. Thus, azide-modified chitosan, or alkyne-modified chitosan are suitable for conjugating to branching units using click chemistry.

In some embodiments, the core moiety is ethylenediamine, or tetra(ethylene oxide). In some embodiments, the core moiety is dipentaerythritol. Exemplary chemical structures suitable for use as core moieties are shown in Table 1 below.

TABLE 1
Structural representation of various building blocks (cores, branching units,
surface functional groups, monomers) for the synthesis of dendrimers.

| Building Blocks | Structure |
|---|---|
| Dipentaerythritol | 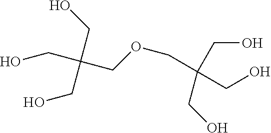 [SMILES: OCC(CO)(CO)COCC(CO)(CO)CO] |
| Pentaerythritol | 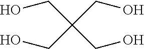 |
| 2-(aminomethyl)-2- (hydroxymethyl) propane-1,3-diol | 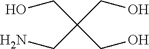 |
| 2-ethyl-2-(hydroxymethyl) propane-1,3-diol | 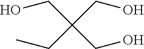 |
| 3,3′,3″,3′′′-silanetetrayltetrakis (propane-1-thiol) | 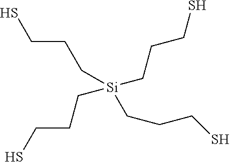 [SMILES: SCCC[Si](CCCS)(CCCS)CCCS] |
| 3,3-divinylpenta-1,4-diene | 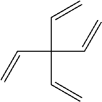 |
| 3,3′,3″-nitrilotripropionic acid | 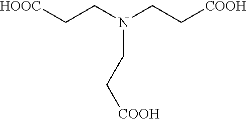 [SMILES: O=C(O)CCN(CCC(=O)O)CCC(=O)O] |
| 3,3′,3″-nitrilotris(N-(2- aminoethyl)propanamide) | 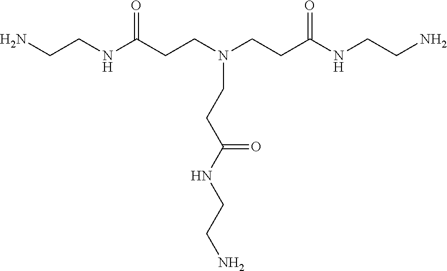 [SMILES: NCCNC(=O)CCN(CCC(=O)NCCN)CCC(=O)NCCN] |
| 3,3′,3″,3′′′-(ethane-1,2- diylbis(azanetriyl)) tetrapropanamide | 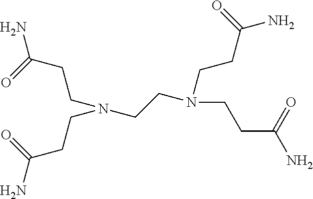 [SMILES: NC(=O)CCN(CCC(N)=O)CCN(CCC(N)=O)CCC(N)=O] |
| 3-(carboxymethyl)-3- hydroxypentanedioic acid | 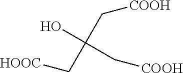 [SMILES: O=C(O)CC(O)(CC(=O)O)CC(=O)O] |
| 2,2′-((2,2-bis((2- hydroxyethoxy)methyl) propane-1,3- diyl)bis(oxy))bis(ethan-1-ol) | 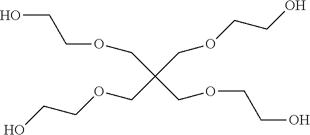 [SMILES: OCCOCC(COCCO)(COCCO)COCCO] |
| tetrakis(3-(trichlorosilyl) propyl)silane | 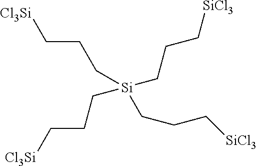 |
| 1-Thioglycerol | 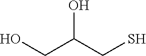 |
| All the sugar based scaffolds including mono, di, tri, or oligomers | 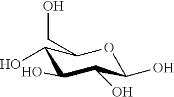 |
| 2,2,4,4,6,6-hexachloro-1,3,5,215, 415,615-triazatriphosphinine | 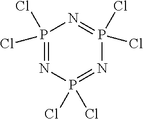 [SMILES: ClP1(Cl)=NP(Cl)(Cl)=NP(Cl)(Cl)=N1] |
| 3-(hydroxymethyl)-5,5- dimethylhexane-2,4-diol | 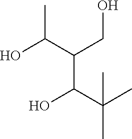 |
| 4,4′,4″-(ethane-1,1,1- triyl)triphenol | 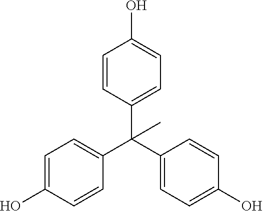 [SMILES: CC(c1ccc(O)cc1)(c1ccc(O)cc1)c1ccc(O)cc1] |
| 2,4,6-trichloro-1,3,5-triazine | 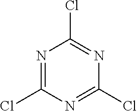 |
| 5-(hydroxymethyl) benzene-1,2,3-triol | 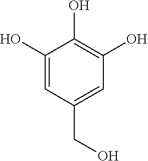 [SMILES: OCc1cc(O)c(O)c(O)c1] |
| 5-(hydroxymethyl)benzene-1,3- diol | 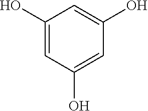 |
| 1,3,5- tris(dimethyl(vinyl)silyl)benzene | 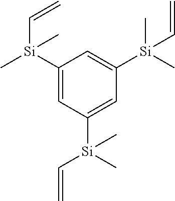 [SMILES: C=C[Si](C)(C)c1cc([Si](C)(C)C=C)cc([Si](C)(C)C=C)c1] |
| Carbosiloxane core | 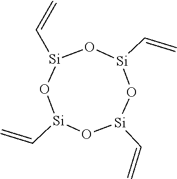 |
| nitrilotrimethanol | 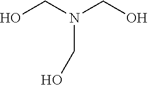 |
| ethylene diamine | 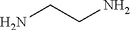 |
| propane-1,3-diamine | 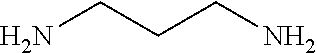 |
| butane-1,4-diamine | 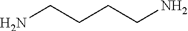 |
| 2,2′,2″-nitrilotris(ethan-1-ol) | 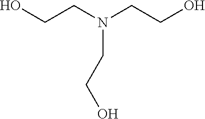 |
| | 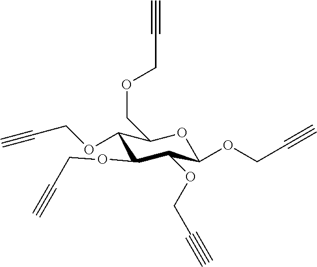 |
| | 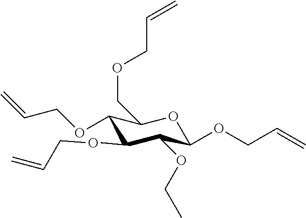 |
| | 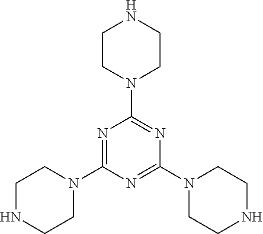 [SMILES: C1CN(c2nc(N3CCNCC3)nc(N3CCNCC3)n2)CCN1] |
| benzene-1,2,3,4,5,6-hexathiol | 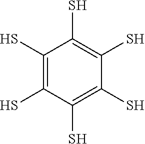 [SMILES: Sc1c(S)c(S)c(S)c(S)c1S] |
| alpha cyclodextrin | 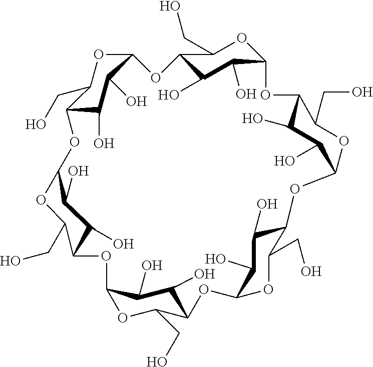 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@H]1[C@H](O)C2O)C(O)[C@H]7O)C(O)[C@H]6O)C(O)[C@H]5O)C(O)[C@H]4O)C(O)[C@H]3O] |
| beta cyclodextrin | 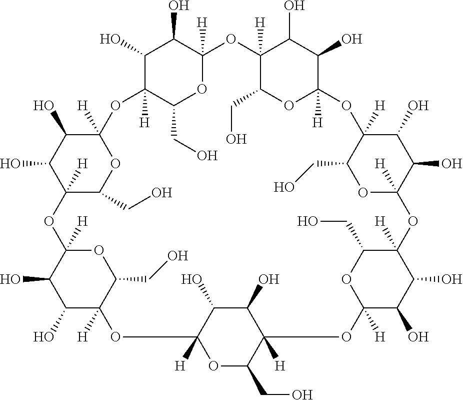 [SMILES: OC[C@H]1O[C@@H]2O[C@H]3C(O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]1[C@H](O)[C@H]2O)O[C@@H]8CO)O[C@@H]7CO)O[C@@H]6CO)O[C@@H]5CO)O[C@@H]4CO)O[C@@H]3CO] |
| gamma cyclodextrin | 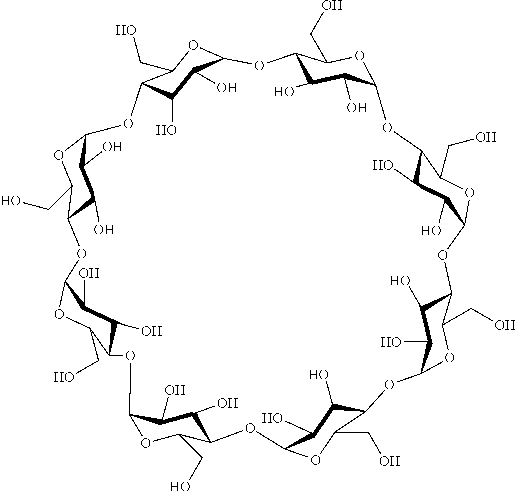 [SMILES: OCC1O[C@@H]2O[C@@H]3C(CO)O[C@H](O[C@@H]4C(CO)O[C@H](O[C@@H]5C(CO)O[C@H](O[C@@H]6C(CO)O[C@@H](O[C@@H]7C(CO)O[C@H](O[C@@H]8C(CO)O[C@H](O[C@@H]9C(CO)O[C@H](O[C@H]1[C@H](O)C2O)C(O)[C@H]9O)C(O)[C@H]8O)C(O)[C@H]7O)C(O)[C@H]6O)C(O)[C@H]5O)C(O)[C@H]4O)C(O)[C@H]3O] |
| Cucurbituril | 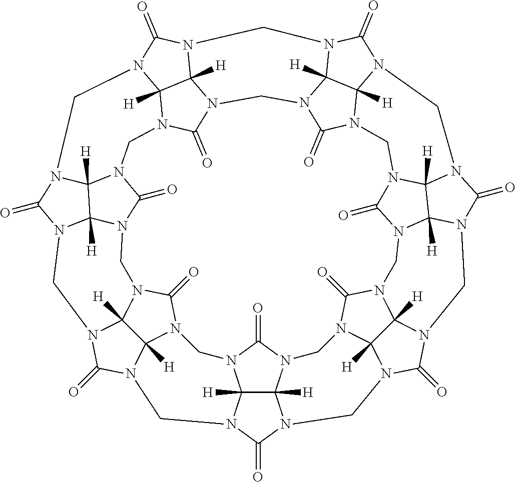 |

Exemplary chemical structures suitable as branching units include monosaccharides. In some embodiments, the monosaccharide branching units are conjugated to the core or the prior layer of monomers via linkers such as polyethylene glycol chains. In preferred embodiments, the monosaccharide branching units are glucose-based branching units. Exemplary glucose-based branching units are shown in Structures II-IV. These are spacer molecules, so can also be alkyl (CH₂)ₙ-hydrocarbon-like units.

The branching units are the PEG or alkyl chain linkers between different dendrimer generations, for example, the glucose layers are connected via PEG linkers and triazole rings.

In preferred embodiments, the branching units are the same for each generation of dendrimers generated from the core. Therefore, in one embodiment, the branching units are glucose-based branching units for generating generation 1 dendrimers as shown in Structures V-VII.

In some embodiments, the branching units are hyper-monomers i.e., ABₙ building blocks. Exemplary hyper-monomers include AB₄, AB₅, AB₆, AB₇, A₈; building blocks. Hyper-monomer strategy drastically increases the number of available end groups. An exemplary AB₄ hypermonomer is peracetylated β-D-Glucopyranoside tetraethylene glycol azide as shown in Structure III.

The chemical structures listed in Table 1, are also suitable as building blocks to form the branching units of the dendrimer. For example, the branching units of the dendrimers are formed by dipentaerythritol, pentaerythritol, 2-(aminomethyl)-2-(hydroxymethyl) propane-1,3-diol, 2-ethyl-2-(hydroxymethyl) propane-1,3-diol, 3,3′,3″,3′″-silanetetrayltetrakis (propane-1-thiol), 3,3-divinylpenta-1,4-diene, 3,3′,3″-nitrilotripropionic acid, 3,3′,3″-nitrilotris(N-(2-aminoethyl) propanamide), 3,3′,3″,3′″-(ethane-1,2-diylbis(azanetriyl)) tetrapropanamide, 3-(carboxymethyl)-3-hydroxypentanedioic acid, 2,2′-((2,2-bis((2-hydroxyethoxy)methyl) propane-1,3-diyl)bis(oxy))bis(ethan-1-ol), tetrakis(3-(trichlorosilyl) propyl)silane, 1-Thioglycerol, 2,2,4,4,6,6-hexachloro-1,3,5,2l5,4l5,6l5-triazatriphosphinine, 3-(hydroxymethyl)-5,5-dimethylhexane-2,4-diol, 4,4′,4″-(ethane-1,1,1-triyl)triphenol, 2,4,6-trichloro-1,3,5-triazine, 5-(hydroxymethyl)benzene-1,2,3-triol, 5-(hydroxymethyl)benzene-1,3-diol, 1,3,5-tris(dimethyl(vinyl)silyl)benzene, Carbosiloxane core, nitrilotrimethanol, ethylene diamine, propane-1,3-diamine, butane-1,4-diamine, 2,2′,2″-nitrilotris(ethan-1-ol), alpha cyclodextrin, beta cyclodextrin, gamma cyclodextrin, Cucurbituril, benzene-1,2,3,4,5,6-hexathiol, monosaccharide, disaccharides, trisaccharides, oligosaccharides, or azide-, alkyne-modified moieties thereof, or a combination thereof.

Other examples of chemical structures that are suitable for forming the branching units of the dendrimers disclosed herein include, but are not limited to, sugar moieties, such as glucose, galactose, mannose, and fructose, and alkylene glycol, such as ethylene glycol, and combinations thereof. In some embodiments, the branching unit is chitosan. Thus, azide-modified chitosan, or alkyne-modified chitosan are suitable for conjugating to the core moiety or additional same or different branching units using click chemistry. In some embodiments, the branching unit is methyl acrylate or ethylenediamine, or a combination thereof. In some embodiments, the branching unit is polyethylene glycerol linear or branched. In some embodiments, the branching unit is a copolymer of an alkylene glycol (such as ethylene glycol) and a sugar moiety, such as glucose, galactose, mannose, and/or fructose.

### 2. Surface Functional Groups

Surface functional groups/molecules of the dendrimers are not limited to a primary amine end group, a hydroxyl end group, a carboxylic acid end group, an acetamide end group, a sugar molecule, an oligo- or poly-alkylene glycol, and/or a thiol end group. In some embodiments, the desired terminal functional groups can be added via one of the conjugation methods for the core and branching unit.

In some embodiments, the surface functional groups are hydroxyl groups, for example those of PAMAM dendrimers, of generation 2 PEG dendrimer as shown in Structure I, or of the terminal glucose of dendrimers prepared with glucose-based branching units as shown in Structures V and VII. In some embodiments, desired surface functional groups can be modified or added via one of the conjugation methods for the core and branching unit. Exemplary surface functional groups include hydroxyl end groups, amine end groups, carboxylic acid end groups, acetamide end group, and thiol end groups, and combinations thereof.

In some embodiments, the dendrimers can specifically target a particular tissue region and/or cell type, such as the cells and tissues of the central nervous system (CNS), the peripheral nervous system (PNS), and/or the eye. In some embodiments, the dendrimers specifically target neurons and/or glia of the CNS. In some embodiments, the dendrimers specifically target neurons and/or glia of the PNS. In some embodiments, the glucose dendrimers are those of generation 1 (G1), G2, G3, G4, and G5, preferably G1, G2, and/or G3.

In some embodiments, the dendrimers include an effective number of terminal glucose and/or hydroxyl groups for targeting to one or more neurons and/or glia of the CNS, the PNS, and/or the eye.

Glucose dendrimers are preferred. In some embodiments, dendrimers are made of glucose and oligoethylene glycol building blocks. Exemplary generation 1 glucose dendrimer is shown in Structure VI, and generation 2 glucose dendrimers is shown in Structure VIII.

In some embodiments, the dendrimers have a plurality of surface functional groups, such as hydroxyl (—OH) groups, amine groups, acetamide groups, and/or carboxyl groups on the periphery of the dendrimers (also referred to herein as surface functional groups or peripheral functional groups). In some embodiments, the surface density of such peripheral functional groups is at least 1 group/nm² (number of the surface functional groups/surface area in nm²). For example, in some embodiments, the surface density of the surface functional groups, such as hydroxyl groups, is more than 0.5, 1, 2, 3, 4, 5, 6, 7, 8, 9, 10 OH groups/nm² such as at least 10, 15, 20, 25, 30, 35, 40, 45, 50, or more than 50 OH group/nm². In some embodiments, the volumetric density of surface functional groups, such as hydroxyl groups, is between about 1 and about 50 groups/nm³, between about 5 and about 30 groups/nm³, or between about 10 and about 20 groups/nm³. In further embodiments, the surface density of the surface functional groups, such as hydroxyl groups, is between about 1 and about 50, preferably 5-20 group/nm² (number of surface functional groups/surface area in nm²) while each surface functional moiety has a molecular weight of between about 100 Da and about 10 kDa, preferably between about 100 Da and 1000 Da.

In some embodiments, the amount of the surface functional groups, such as any one of those described above, e.g., hydroxyl groups, of the dendrimer is at least 40%, at least 50%, more than 40%, more than 50%, or in a range from more than 40% to 100%.

In some embodiments, one or more of the surface functional groups, such as any one of those described above, on the periphery of the dendrimers are further modified by conjugating with one or more carbohydrate molecules and/or more or more polyalkylene glycols, such as polyethylene glycols. In these embodiments, the surface density of the terminal carbohydrate moieties/molecules and/or polyalkylene glycols can be in any of the ranges described above for hydroxyl groups. Hydroxyl-terminated PAMAM dendrimers, PAMAM dendrimer modified on the surface with sugar moieties with >10% of surface groups modified by sugars, especially by glucose, and glucose dendrimers (where the dendrimers are made of glucose building blocks) are preferred. For delivery to the brain, constructs with a total molecular weight of <30,000 Da are preferred. For confinement primarily to the peripheral circulation, constructs with a total molecular weight of >50,000 Da are preferred. When dendrimers are formed of, or include sugar moieties/molecules at termination, for example, glucose, the terminal hydroxyl groups of these dendrimers may be part of the terminal sugar moieties/molecules or extra hydroxyl groups that are not part of the sugar moieties/molecules, or a combination thereof. In some embodiments, all of the terminal hydroxyl groups are part of the terminal sugar moieties/molecules.

a. Hydroxyl-terminated Dendrimers

In some embodiments, the dendrimers include a plurality of hydroxyl groups. Some exemplary high-density hydroxyl groups-containing dendrimers include commercially available polyester dendritic polymer such as hyperbranched 2,2-Bis (hydroxyl-methyl) propionic acid polyester polymer (for example, hyperbranched bis-MPA polyester-64-hydroxyl, generation 4), dendritic polyglycerols. In some embodiments, the hydroxyl terminated dendrimers include hydroxyl-terminated PAMAM dendrimers, particularly G3 to G6 hydroxyl-terminated PAMAM dendrimers, such as G3, G4, G5, and G6 hydroxyl-terminated PAMAM dendrimers.

In some embodiments, the high-density hydroxyl groups-containing dendrimers are oligo ethylene glycol (OEG)-like dendrimers. For example, a generation 2 OEG dendrimer (D2-OH-60) as shown in Structure I can be synthesized using highly efficient, robust and atom economical chemical reactions such as Cu (I) catalyzed alkyne-azide click and photo catalyzed thiol-ene click chemistry. Highly dense polyol dendrimer at very low generation in minimum reaction steps can be achieved by using an orthogonal hypermonomer and hypercore strategy, for example as described in WO2019094952. In some embodiments, the dendrimer backbone has non-cleavable polyether bonds throughout the structure to avoid the disintegration of dendrimer in vivo and to allow the elimination of such dendrimers as a single entity from the body (non-biodegradable).

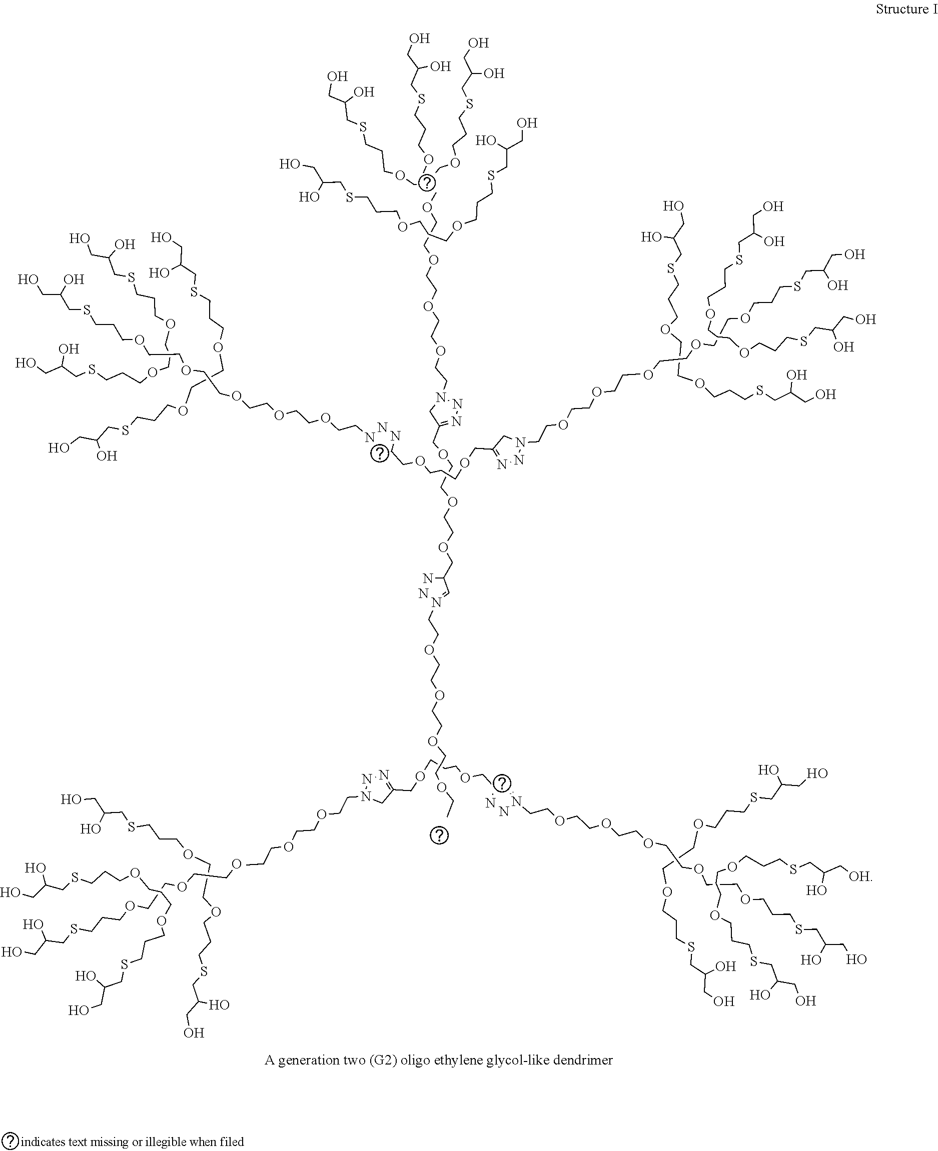

In some embodiments, the dendrimers have a plurality of hydroxyl (—OH) groups on the periphery of the dendrimers. In some embodiments, the surface density of hydroxyl (—OH) groups is at least 1 OH group/nm² (number of surface hydroxyl groups/surface area in nm²). For example, in some embodiments, the surface density of hydroxyl groups, per nm², is more than 0.5, 1, 2, 3, 4, 5, 6, 7, 8, 9, 10 OH groups/nm², such as at least 10, 15, 20, 25, 30, 35, 40, 45, 50, or more than 50. In some embodiments, the volumetric density of hydroxyl groups is between about 1 and about 50 groups/nm³, between about 5 and about 30 groups/nm³, or between about 10 and about 20 groups/nm³. In further embodiments, the surface density of hydroxyl (—OH) groups is between about 1 and about 50, or between 5 and 20 OH group/nm² (number of surface hydroxyl groups/surface area in nm²) while having a molecular weight of between about 100 Da and about 10 kDa, preferably between about 100 Da and 1000 Da. In some embodiments, the amount of the surface hydroxyl groups of the dendrimer is preferably greater than 35%, at least 40%, at least 50%, more than 40%, more than 50%, or in a range from more than 40% to 100%. In some embodiments, the dendrimers may have a fraction of the hydroxyl groups exposed on the outer surface, with the others in the interior core of the dendrimers.

In some embodiments, the dendrimer specifically targets a particular tissue region and/or cell types following administration into the body. In some embodiments, the dendrimer specifically targets a particular tissue region and/or cell type without a targeting moiety. In some embodiments, the dendrimers include an effective number of hydroxyl groups for targeting CNS cells and/or PNS cells, such as microglial, astrocytes, and/or neurons associated with a disease, disorder, or injury of the central nervous system or the peripheral nervous system. In some embodiments, the dendrimer specifically targets a particular tissue region and/or cell type without a targeting moiety and the active agent conjugated thereto bind directly to a receptor on the surface and/or interior of target neural and/or glial cells.

In some embodiments, the dendrimers are able to specifically target a particular tissue region and/or cell type, preferably the cells and tissues of the central nervous system (CNS) and the eye. In some embodiments, the dendrimers specifically target neurons of the CNS and the eye. Unmodified PAMAM dendrimers with hydroxyl end groups do not enrich in the neurons of brain and/or retinal ganglion cells (RGCs) in the eye as much as these glucose dendrimers. The glucose dendrimers with terminal glucose monosaccharide and a high density of hydroxyl functional groups effectively target the neurons in a generation dependent manner. Examples demonstrate efficacy with generation 2 (G2), and G3 and G4 should be efficacious. G5 and above are more difficult to use.

In preferred embodiments, the dendrimers include an effective number of terminal glucose and/or hydroxyl groups for targeting to one or more neurons of the CNS, or the eye. The hydroxyl groups on the dendrimer surface are part of glucose molecules. There are no extra hydroxyls in addition to the glucose molecules on the surface. The number of sugar molecules on the surface is determined by the generation number. All generations are expected to target neurons.

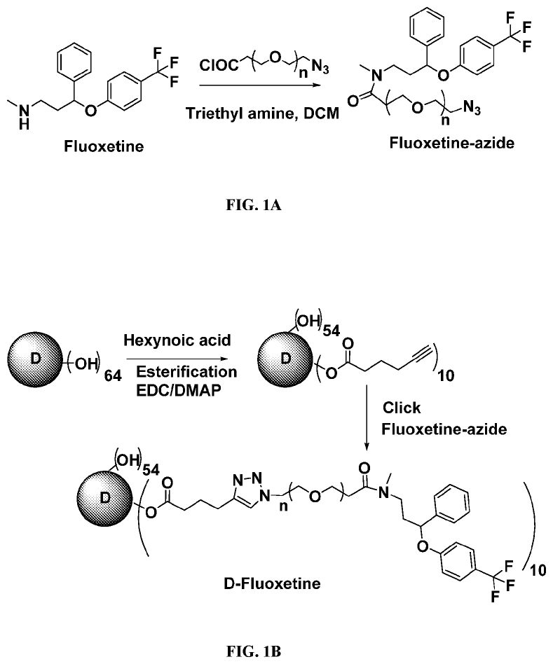

In some embodiments, dendrimers are made of glucose and oligoethylene glycol building blocks. Exemplary glucose dendrimers are shown in the Examples, for example, generation 1 dendrimers as shown in Structures IV-VI, and generation 2 dendrimers as shown in FIGS. 1A and 1B. Some exemplary glucose dendrimers include a generation 1 glucose dendrimer having 24 hydroxyl (—OH) end groups, a generation 2 glucose dendrimer having 96 hydroxyl (—OH) end groups, a generation 3 glucose dendrimer having 396 hydroxyl (—OH) end groups, and generation 4 glucose dendrimer having 1584 hydroxyl (—OH) end groups. In a preferred embodiment, the glucose dendrimer is a generation 2 glucose based dendrimer that has 24 glucose molecules at the periphery and 6 embedded glucose molecules in the backbone held together by PEG segments.

b. Dendrimers Modified with Carbohydrates

In some embodiments, the dendrimers contain one or more carbohydrate molecules at the termination. These terminal carbohydrate molecules can be prepared by conjugating one or more surface functional groups of a dendrimers, such as amine groups, carboxyl groups, or hydroxyl groups, with one or more carbohydrate molecules. In preferred embodiments, the dendrimers, prior to carbohydrate conjugation, are hydroxyl-terminated dendrimers such as hydroxyl-terminated PAMAM dendrimers and one or more of the hydroxyl groups are conjugated with one or more carbohydrate molecules.

In some embodiments, hydroxyl-terminated dendrimers modified with surface glucose molecules selectively target central and/or peripheral neural and/or glial cells in vitro and in vivo; and/or selectively accumulate on the surface and/or within these target neural cells, glial cells, and/or macrophage cells, such that the active agent(s) conjugated thereto bind to one or more receptors on/in the target neural and/or glial cells.

In some embodiments, the carbohydrate moieties used to modify one or more surface functional groups of the dendrimers are monosaccharides. Exemplary monosaccharides suitable for modifying the dendrimers include glucose, glucosamine, galactose, mannose, fructose, dehydroascorbic acid, urate, myo-inositol. In some embodiments, the dendrimers are conjugated to glucose and thus contain glucose as terminal moieties/molecules. In some embodiments, hydroxyl-terminated dendrimers are modified with one or more glucose moieties to the dendrimer (“D-Glu”). In some embodiments, the dendrimers are conjugated to galactose. In some embodiments, the dendrimers are conjugated to mannose. In some embodiments, the dendrimers are conjugated to fructose. In some embodiments, the dendrimers are conjugated to one or more monosaccharides other than glucose, such as galactose, mannose, and/or fructose. For example, the carbohydrate moieties are oligosaccharides which terminate in one or more monosaccharides including glucose, glucosamine, mannose, fructose, thus exposing these sugar moieties on the surface for binding.

The glucose dendrimers or glucose-modified dendrimers are used to obtain selective uptake by the target cells. The drug conjugated to the dendrimer binds to receptors or other sites of action. In preferred embodiments, the dendrimers (e.g., glucose or hydroxyl-terminated PAMAM dendrimers) or carbohydrate-functionalized dendrimers, are conjugated to one or more carbohydrates moietiesactive agents that have affinity to and are suitable for binding one or more of serotonin (5HT) receptors e.g., 5HT-1A, 5HT-2B, 5HT-2A, 5HT-2B, 5HT-2C, 5HT-3, 5HT-4. In some embodiments, the dendrimers are conjugated to one or more carbohydrates moieties that have affinity to and are suitable for binding one or more norepinephrine (NE) receptors e.g., α1A adrenergic receptor, α1B-adrenergic receptor, α1D-adrenergic receptor, α2A-adrenergic receptor, α2B-adrenergic receptor, α2C-adrenergic receptor, β1-adrenergic receptor, and β2-adrenergic receptor. In some embodiments, the dendrimers are conjugated to one or more carbohydrates moieties that have affinity to and are suitable for binding directly or indirectly, dopamine D1 and D2 receptors. In some embodiments, the dendrimers are conjugated to one or more carbohydrates moieties that have affinity to and are suitable for binding one or more non-cannabinoid receptors such as G-protein coupled receptors e.g., GPR55, GPR18, GPR3, GPR6, GPR12 GPR40, GPR43, GPR41, GPR120, GPR23, GPR92, GPR84, GPR119, or GPR35; the adenosine receptor such as adenosine A3; the muscarinic acetylcholine receptors e.g., M1 and M4; the serotonin receptors e.g., 5-HT1A, 5-HT2A; opioid receptors e.g., μ- and δ-opioid receptors; and tachykinin NK2 receptors. In some embodiments, the dendrimers are conjugated to one or more carbohydrates moieties, or made of sugar moieties, that have affinity to and are suitable for transport via one or more of GLUT1, GLUT2, GLUT3, GLUT4, GLUT5, GLUT6, GLUT7, GLUT8, GLUT9, GLUT10, GLUT11, GLUT12, GLUT13, and GLUT14. In further embodiments, the dendrimers are conjugated to one or more glucose and/or glucosamine moieties. In other embodiments, the dendrimers are conjugated to one or more oligosaccharides terminating in glucose and/or glucosamine moieties, i.e., glucose and/or glucosamine moieties are exposed on the surface of the dendrimer conjugates suitable for binding to one or more of the GLUTs, GLUTs, 5HT receptors, DA receptors, NE receptors and/or transporters.

In some embodiments, the dendrimers have a plurality of carbohydrate moieties/molecules such as monosaccharides, e.g., glucose, on the periphery of the dendrimers, or have sugar building blocks for the dendrimers. In some embodiments, the surface density of carbohydrate molecules such as monosaccharides, e.g., glucose, is at least 1 carbohydrate molecule/nm² (number of surface carbohydrate groups/surface area in nm²). In some embodiments, the surface density of carbohydrate molecules, per nm², is more than 0.5, 1, 2, 3, 4, 5, 6, 7, 8, 9, 10 OH groups/nm², such as at least 10, 15, 20, 25, 30, 35, 40, 45, 50, or more than 50. For example, surface density of carbohydrate molecules, per nm², is more than 10. In some embodiments, the volumetric density of surface carbohydrate molecules is between about 1 and about 50 groups/nm³, between about 5 and about 30 groups/nm³, or between about 10 and about 20 groups/nm³. In further embodiments, the surface density of carbohydrate molecules is between about 1 and about 50, between about 5 and about 20, per nm² (number of surface carbohydrate molecules/surface area in nm²) while each carbohydrate moiety having a molecular weight of between about 100 Da and about 1000 Da. In these embodiments, i.e., one or more surface functional groups of the dendrimer are modified to introduce one or more sugar moieties/molecules at termination, the terminal hydroxyl groups may be part of the terminal sugar moieties/molecules or extra hydroxyl groups that are not modified with sugar moieties/molecules and thus are not part of the sugar moieties/molecules, or a combination thereof.

In some embodiments, carbohydrate molecules such as monosaccharides, e.g., glucose, are present in an amount by weight that is between about 1% and 40% of the total weight of the glycosylated dendrimer, for example, between about 2% and 20%, between about 5% and 15%, or between 9% and 12% of the total weight of the glycosylated dendrimer. For example, in some embodiments, the carbohydrate moieties are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the glycosylated dendrimer following conjugation. In some embodiments, conjugation of carbohydrate molecules through one or more surface functional groups occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of carbohydrate molecules occurs on less than 5%, less than 10%, less than 15%, less than 20%, less than 25%, less than 30%, less than 35%, less than 40% of total available surface functional groups of the dendrimers prior to the conjugation.

c. Dendrimers Modified with Polyalkylene Glycol

In some embodiments, the dendrimers contain one or more polyalkylene glycols at the termination. These terminal polyalkylene glycols can be prepared by conjugating one or more of surface functional groups of the dendrimers, such as hydroxyl groups, with a polyalkylene glycol, such as PEG. In some embodiments, the dendrimers, prior to conjugation, are hydroxyl-terminated dendrimers such as hydroxyl-terminated PAMAM dendrimers and at least a portion of the surface hydroxyl groups are conjugated with PEG.

In some embodiments, the dendrimers have a plurality of polyalkylene glycols such as PEG, on the periphery of the dendrimers. In some embodiments, the surface density of polyalkylene glycols such as PEG, is at least 1 polyalkylene glycol/nm² (number of surface polyalkylene glycol/surface area in nm²). In some embodiments, the surface density of polyalkylene glycols, per nm², is more than 0.5, 1, 2, 3, 4, 5, 6, 7, 8, 9, 10, such as at least 10, 15, 20, 25, 30, 35, 40, 45, 50, or more than 50. For example, surface density of polyalkylene glycols, per nm², is more than 10. In some embodiments, the volumetric density of surface polyalkylene glycols is between about 1 and about 50 groups/nm³, between about 5 and about 30 groups/nm³, or between about 10 and about 20 groups/nm³. In further embodiments, the surface density of polyalkylene glycols such as PEG is between about 1 and about 50, between about 5 and about 20, per nm² (number of surface polyalkylene glycols/surface area in nm²) while having a molecular weight of between about 100 Da and about 10 kDa.

In some embodiments, the polyalkylene glycol molecules such as PEG can be present in an amount by weight that is between about 1% and 40% of the total weight of the pegylated dendrimer, for example, between about 2% and 20%, between about 5% and 15%, or between 9% and 12% of the total weight of the pegylated dendrimer. For example, in some embodiments, the polyalkylene glycol molecules, such as PEG, are present in an amount that is about 1%, 2%, 3%, 4%, 5%, 6%, 7%, 8%, 9%, 10%, 11%, 12%, 13%, 14%, 15%, 16%, 17%, 18%, 19%, or 20% of the total weight of the pegylated dendrimer following conjugation.

In some embodiments, conjugation of polyalkylene glycol molecules such as PEG through one or more surface functional groups of the dendrimer occurs via about 5%, 6%, 7%, 8%, 9%, 10%, 15%, 20%, or 25% of the total available surface functional groups, preferably hydroxyl groups, of the dendrimers prior to the conjugation. In other embodiments, the conjugation of polyalkylene glycol molecules such as PEG occurs on less than 5%, less than 10%, less than 15%, less than 20%, less than 25%, less than 30%, less than 35%, less than 40% of total available surface functional groups of the dendrimers prior to the conjugation.

### B. Antidepressant and Antipsychotic Agents

The dendrimers are complexed to or covalently conjugated to one or more antidepressant and antipsychotic agents. Exemplary antidepressant and antipsychotic agents include, but are not limited to, a range of drug classes including selective serotonin reuptake inhibitors (SSRIs), serotonin-norepinephrine reuptake inhibitors (SNRIs), antipsychotics, atypical antipsychotics, tricyclic antidepressants (TCAs), monoamine oxidase inhibitors (MAOIs), benzodiazepines, beta-blockers (BB), and anticonvulsants. The antidepressant antipsychotic agents, and their derivatives typically bind to one or more receptors, thereby modulating signaling in neurotransmitter signaling in the central nervous system. Antidepressants typically inhibit reuptake of neurotransmitters, particularly serotonin, dopamine, and noradrenaline, through selective receptors thereby increasing the concentration of these specific neurotransmitters in the synaptic cleft. Antipsychotics typically reduce neurotransmission in dopamine pathways by either D2 antagonism or partial D2 receptor agonism. Thus, partial antagonism, functional selectivity and inverse agonism all play important roles in determining the cellular response to specific neurotransmitter receptor ligands.

### 1. Antidepressants

a. Selective Serotonin Reuptake Inhibitors (SSRIs)

The compositions may include a dendrimer complexed to one or more SSRIs. “SSRIs” or “Selective Serotonin Reuptake Inhibitors” are a class of drugs that are typically used as antidepressants in the treatment of major depressive disorder and anxiety disorders. SSRIs are believed to increase the extracellular level of the neurotransmitter serotonin by limiting its reabsorption (reuptake) into the presynaptic cell, increasing the level of serotonin in the synaptic cleft available to bind to the postsynaptic receptor.

SSRIs are known to selectively inhibit serotonin transport by inhibiting serotonin reuptake, particularly by binding to the Serotonin Transporter (SERT). This action of SSRIs results in abrupt increase in serotonin in the somatodendritic area of serotonergic neurons leading to desensitization of the somatodendritic serotonin-1A autoreceptors. Consequently, the neuronal impulse flow is increased. It causes increased release of serotonin from axon terminals, which culminates into desensitization of postsynaptic serotonin receptors. Desensitization of these receptors may contribute to the therapeutic actions of SSRIs or could account for the development of tolerance to acute side effects of SSRIs. SSRIs cause strong but slow disinhibition of 5-HT neurotransmission in the central nervous system (CNS) and peripheral nervous system (PNS). In this case, the actions of antidepressants are mediated by a pathway from midbrain raphe to prefrontal cortex. Since a majority of the serotonin in the human body is produced within the gut, SSRIs affect enteric neurons, thereby increasing serotonin availability, increasing enteric neurogenesis and faster in vitro GI motility. In fact, serotonin receptors are widely expressed within the GI tract, and five of the seven known families, 5-HT1, 5-HT2, 5-HT3, 5-HT4, and 5-HT7 receptors, are expressed in the gut and can affect gut functions (Mawe and Hoffman (2013), Nat Rev Gastroenterol Hepatol. 2013 October; 10 (10): 564).

In some forms, one or more SSRIs may be conjugated to the dendrimer including but not limited to fluoxetine (Prozac™), paroxetine (Paxil™), sertraline (Zoloft™), citalopram (Celexa™), fluvoxamine (Luvox™), escitalopram (Lexapro™). In other forms, the SSRI-like agents that may be included in the composition include, but is not limited to, one or more of Dapoxetine (Priligy), R-fluoxetine, Indalpine (Upstène), Zimelidine (Zelmid), Alaproclate (GEA-654), Centpropazine, Cericlamine (JO-1017), Femoxetine (Malexil; FG-4963), Ifoxetine (CGP-15210), Omiloxetine, Panuramine (WY-26002), Pirandamine (AY-23713), and Seproxetine ((S)-norfluoxetine).

In some forms, the compositions may include one or more SSRI-related compounds in an amount effective to deliver a low dose of the SSRI-related compound. For example, although described as SNRIs, duloxetine (Cymbalta), venlafaxine (Effexor), and desvenlafaxine (Pristiq) may selectively act as serotonin reuptake inhibitors (SRIs). They are about at least 10-fold selective for inhibition of serotonin reuptake over norepinephrine reuptake. The selectivity ratios are about 1:30 for venlafaxine, about 1:10 for duloxetine, and about 1:14 for desvenlafaxine. At low doses, these SNRIs act as SSRIs; only at higher doses do they also prominently inhibit norepinephrine reuptake. In another example, Milnacipran (Ixel, Savella) and its stereoisomer levomilnacipran (Fetzima) are the only widely marketed SNRIs that inhibit serotonin and norepinephrine to similar degrees, both with ratios close to 1:1. In a third example, Vilazodone (Viibryd) and vortioxetine (Trintellix) are SRIs that also act as modulators of serotonin receptors and are described as serotonin modulators and stimulators (SMS). Vilazodone is a 5-HT1A receptor partial agonist while vortioxetine is a 5-HT1A receptor agonist as well as a 5-HT3 and 5-HT7 receptor antagonist. Litoxetine (SL 81-0385) and lubazodone (YM-992, YM-35995) are SRIs, with litoxetine being a 5-HT3 receptor antagonist while lubazodone is also a 5-HT2A receptor antagonist.

In preferred embodiments, the dendrimer is conjugated to one or more SSRIs or SSRI derivatives as shown by the Structures I-VI below.

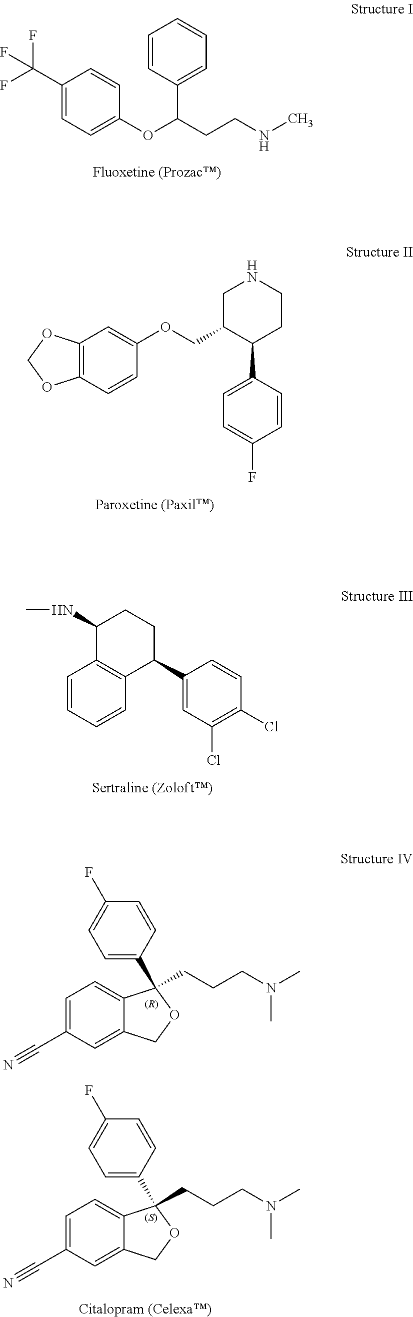

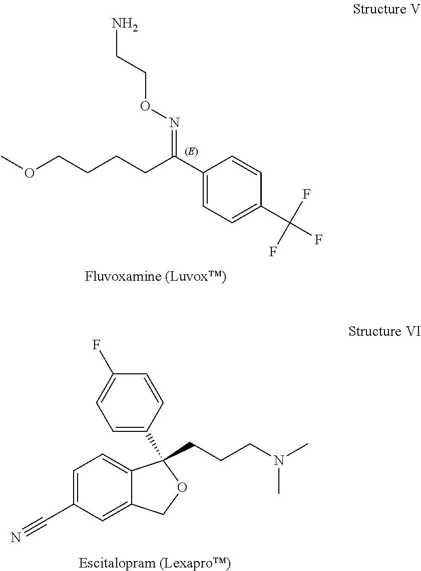 [SMILES: COCCCC/C(=N\OCCN)c1ccc(C(F)(F)F)cc1 ; CN(C)CCC[C@@]1(c2ccc(F)cc2)OCc2cc(C#N)ccc21]

b. Serotonin-norepinephrine reuptake inhibitors (SNRIs)

The compositions may include a dendrimer complexed to one or more SNRIs. By “SNRI” is meant any member of the class of compounds that act upon, and increase, the levels of two monoamine neurotransmitters in the brain, namely serotonin and norepinephrine (Arroll B, et al., Annals of Family Medicine, 2005; 3(5):449-456). SNRIs exert their action by (i) inhibiting the pre-synaptic reuptake of serotonin from the synaptic cleft by binding to the SERT receptors and (ii) inhibiting the pre-synaptic reuptake of norepinephrine from the synaptic cleft by binding to the norepinephrine (NAT) transporter. Noradrenaline (norepinephrine, NE) is found in cell bodies of the pons and medulla. These cell bodies project neurons to the hypothalamus, thalamus, limbic system, locus coeruleus, and lateral tegmental, and cerebral cortex. In the peripheral nervous system, noradrenaline is used as a neurotransmitter by sympathetic ganglia located near the spinal cord or in the abdomen, as well as Merkel cells located in the skin. NE is also released directly into the bloodstream by the adrenal glands, and abdominal viscera & sphincter contraction of the GI tract and urinary bladder. Prevention of reuptake prolongs the persistence of these monoamines in the synaptic cleft within the central nervous system (CNS) and the peripheral nervous system (PNS). Accordingly, this results in increased postsynaptic receptor stimulation and additional post synaptic neuronal transmission.

SNRIs are often considered to have a ‘dual action’ on account of their mechanism, sometimes referred to as non-tricyclic serotonin and norepinephrine reuptake inhibitors. However, the specific degree of reuptake inhibition of norepinephrine and serotonin is both dose- and agent-dependent. Suitable SNRIs that may be conjugated to the dendrimer compositions include but are not limited to Venlafaxine (Effexor XR™), Desvenlafaxine (Pristiq and Khedezla™), Duloxetine (Cymbalta, Irenka), Milnacipran (Savella), Levomilnacipran (Fetzima), Tramadol HCL, Sibutramine (Meridia), Atomoxetine (Strattera), and Bicifadine (DOV-220,075), and their derivatives.

In some forms, the SNRI conjugated to the dendrimer has a stronger binding affinity for serotonin receptors compared to norepinephrine receptors. For example, duloxetine and venlafaxine preferentially bind serotonin receptors compared to norepinephrine receptors, thereby exerting increased potency for serotonin reuptake relative to norepinephrine reuptake. In some forms, the SNRI conjugated to the dendrimer has a stronger binding affinity for norepinephrine receptors compared to serotonin receptors. In some forms, the SNRI conjugated to the dendrimer has equal affinity for binding serotonin receptors and norepinephrine receptors.

In some forms, the SNRI conjugated to the dendrimer indirectly modulates signaling at a non-serotonin and non-epinephrine receptor. In an exemplary embodiment, the dendrimer can be conjugated to venlafaxine, a synthetic phenethylamine bicyclic derivative and a substrate of P-glycoprotein (P-gp), which pumps it out of the brain. At high doses, venlafaxine reduces the pre-synaptic reuptake of dopamine, thereby enhancing dopamine levels, particularly in the prefrontal cortex. The mechanism of action behind the increase in dopamine levels involves the inhibition of norepinephrine transporters. These transporters have a significant affinity for dopamine, resulting in the transporter's ability to act on both dopamine and norepinephrine. Therefore, inhibition of norepinephrine transporters can lead to an increase in dopamine. This increase in dopamine specifically occurs in the prefrontal cortex, where dopamine transporters are scarce, and reuptake relies more heavily on norepinephrine transporters. Venlafaxine also indirectly modulates opioid receptors, muscarinic acetylcholine receptors (mAChR), histaminergic receptors, as well as the α1 and α2-adrenergic receptors.

In some embodiments, the dendrimer is conjugated to one or more SNRIs or derivatives thereof, in an amount effective to provide to the cells between about 40 mg/day to about 60 mg/day, between about 60 mg/day to about 80 mg/day, 80 between about 40 mg/day to about 100 mg/day, between about 100 mg/day to about 150 mg/day, between about 150 mg/day to about 200 mg/day, between about 200 mg/day to about 250 mg/day, 250-300 mg/day, between about 350 mg/day to about 450 mg/day of the one or more SNRIs or derivatives thereof.

In some embodiments, dendrimers conjugated to SSRIs may exert anti-inflammatory effects on certain cells which may improve treatment efficacy of mental health and neurological disorders.

In some embodiments, conjugation of SSRIs to dendrimers resulting in improved pharmacokinetics and receptor targeting, may also significantly reduce the time required for onset of action.

In preferred embodiments, the dendrimer is conjugated to one or more SSRIs or SSRI derivatives as shown by the Formulas VIII-XI below.

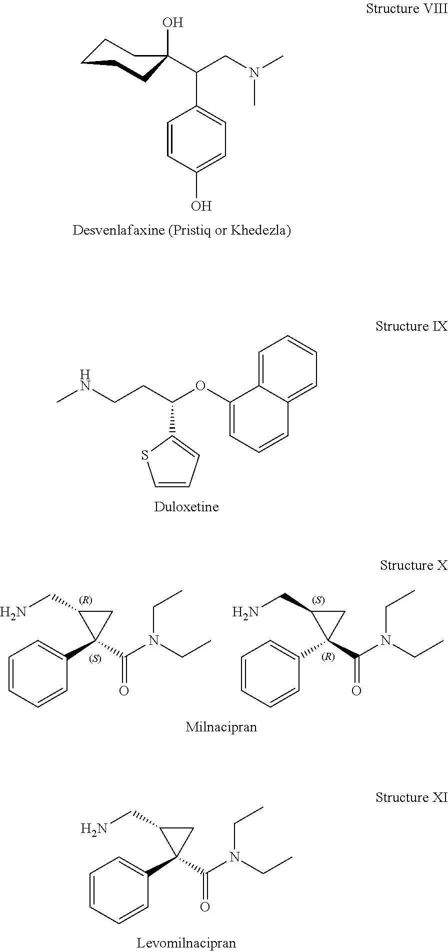

c. Atypical Antidepressants

The compositions may include a dendrimer complexed to one or more atypical antidepressants. By “atypical antidepressant” is meant any antidepressant agent that acts in a manner that is different from that of most other antidepressant agents. Atypical antidepressants are frequently used in patients with major depression who have inadequate responses or intolerable side effects during first-line treatment with SSRIs. Suitable atypical antidepressants that can be conjugated to the dendrimers include, but are not limited to, agomelatine, bupropion, mianserin, mirtazapine, nefazodone, opipramol, tianeptine, and trazodone.

In an exemplary embodiment, the dendrimers can be complexed to agomelatine. Agomelatine is a receptor agonist for the melatonin MT1 and MT2 receptors as well as a receptor antagonist for the serotonin 5-HT2C and 5-HT2B receptors. By antagonizing 5-HT2C, it disinhibits/increases noradrenaline and dopamine release specifically in the frontal cortex. Therefore, it is sometimes classified as a norepinephrine-dopamine disinhibitor. Antagonism of 5-HT2B is putatively an antidepressant property agomelatine shares with several atypical antipsychotics, such as aripiprazole.

In a second exemplary embodiment, the dendrimers can be complexed to mirtazapine. Mirtazapine has a dual mode of action. It is a noradrenergic and specific serotonergic antidepressant (NaSSA) that acts by antagonizing the adrenergic alpha2-autoreceptors and alpha2-heteroreceptors as well as by blocking 5-HT2 and 5-HT3 receptors. Therefore, mirtazapine enhances the release of norepinephrine and 5-HT1A-mediated serotonergic transmission. Mirtazapine is extensively metabolized in the liver by the cytochrome (CYP) P450 isoenzymes CYP1A2, CYP2D6, and CYP3A4.

In a third exemplary embodiment, the dendrimers can be complexed to nefazodone. Nefazodone is a phenylpiperazine compound structurally related to trazodone and is described as a serotonin antagonist and reuptake inhibitor (SARI). Nefazodone acts primarily as a potent antagonist of the serotonin 5-HT2A receptor and to a lesser extent of the serotonin 5-HT2C receptor. It also has high affinity for the al-adrenergic receptor and serotonin 5-HT1A receptor, and relatively lower affinity for the α2-adrenergic receptor and dopamine D2 receptor. Nefazodone has significant affinity for the serotonin, norepinephrine, and dopamine transporters as well, and therefore acts as a weak serotonin-norepinephrine-dopamine reuptake inhibitor (SNDRI).

In a fourth exemplary embodiment, the dendrimers can be complexed to trazodone. Trazodone is a mixed agonist and antagonist of various serotonin receptors, antagonist of adrenergic receptors, weak histamine H1 receptor antagonist, and weak serotonin reuptake inhibitor. In some forms, Trazodone is an antagonist of 5-HT2A and 5-HT2B receptors, a partial agonist of the 5-HTIA receptor, and an antagonist of the α1- and α2-adrenergic receptors.

In another exemplary embodiment, the dendrimers can be complexed to bupropion, a dopamine and noradrenaline reuptake inhibitor. Bupropion reduces the pre-synaptic reuptake of dopamine and noradrenaline, and acts as an antagonist of several nicotinic acetylcholine receptors. Bupropion is generally metabolized to three known active metabolites, hydroxybupropion (R,R-Hydroxybupropion and S,S-Hydroxybupropion) and the diasteroisomers, threohydrobupropion and erythrohydrobupropion.

In yet another exemplary embodiment, the dendrimers can be complexed to mianserin. Mianserin is a tetracyclic antidepressant that has antihistaminic and hypnosedative, but almost no anticholinergic, effect. Mianserin appears to exert its effects via antagonism of histamine and serotonin receptors, and inhibition of norepinephrine reuptake. More specifically, mianserin is an antagonist/inverse agonist at most or all sites of the histamine H₁ receptor, serotonin 5-HT_{1D}, 5-HT_{1F}, 5-HT_{2A}, 5-HT_{2B}, 5-HT_{2C}, 5-HT₃, 5-HT₆, and 5-HT₇ receptors, and adrenergic α1- and α2-adrenergic receptors, and additionally a norepinephrine reuptake inhibitor. As an H₁ receptor inverse agonist with high affinity, mianserin has strong antihistamine effects (e.g., sedation). Conversely, it has low affinity for the muscarinic acetylcholine receptors, and hence lacks anticholinergic properties. Mianserin has been found to be a low affinity but potentially significant partial agonist of the κ-opioid receptor.

d. Tricyclic Antidepressants (TCAs)

The compositions may include a dendrimer complexed to one or more tricyclic antidepressants. By “cyclic antidepressant” is meant any antidepressant agent designated as tricyclic or tetracyclic, depending on the number of rings in their chemical structure—three (tri) or four (tetra). Tricyclic antidepressants act on approximately five different neurotransmitter pathways to achieve their effects (Stahl et al., Prim. Care Companion J. Clin. Psychiatry 2004; 6(4):159). Tricyclic antidepressants block the reuptake of serotonin and norepinephrine in presynaptic terminals, which leads to increased concentration of these neurotransmitters in the synaptic cleft. The increased concentrations of norepinephrine and serotonin in the synapse may contribute to the anti-depressive effect of tricyclic antidepressants. Additionally, in some forms, they may act as competitive antagonists on post-synaptic alpha cholinergic (alpha1 and alpha2), muscarinic, and histaminergic receptors (H1). The structure of the receptor greatly influences the binding affinity of the TCA.

The chemical structure of a TCA consists of a three-ringed structure with an attached secondary or tertiary amine. Secondary amines include desipramine, nortriptyline, and protriptyline, while tertiary amines consist of amitryptiline, clomipramine, doxepin, imipramine, and trimipramine. Tertiary amines tend to have greater blockage of serotonin reuptake, while secondary amines have greater blockage of norepinephrine uptake. The combination of different amine structures and variations in chemical composition contribute to the multitude of adverse effects seen with TCA usage as these factors affect TCA-receptor affinity and binding.

Suitable tricyclic antidepressants that can be conjugated to the dendrimers include but are not limited to Amitriptyline (e.g., Elavil, Endep), Amitriptylinexide (e.g., Amioxid, Ambivalon, Equilibrin), Clomipramine (e.g., Anafranil), Desipramine (e.g., Norpramin, Pertofrane), Dibenzepin (e.g., Noveril, Victoril), Dimetacrine (e.g., Istonil), Dosulepin (e.g., Prothiaden), Doxepin (e.g., Adapin, Sinequan), Imipramine (e.g., Tofranil), Lofepramine (e.g., Lomont, Gamanil), Amoxapine, Melitracen (e.g., Dixeran, Melixeran, Trausabun), Nitroxazepine (e.g., Sintamil), Nortriptyline (e.g., Pamelor, Aventyl), Noxiptiline (e.g., Agedal, Elronon, Nogedal), Opipramol (e.g., Insidon), Pipofezine (e.g., Azafen/Azaphen), Protriptyline (e.g., Vivactil), and/or Trimipramine (e.g., Surmontil).

Suitable tetracyclic antidepressants that can be conjugated to the dendrimers include but are not limited to Maprotiline (Ludiomil; can also be classified as a TCA and grouped with the secondary amines), Mianserin (Tolvon), Mirtazapine (Remeron), Setiptiline (Tecipul), Amoxapine (Asendin; often classified as a TCA and grouped with the secondary amines), Quetiapine (Seroquel; an atypical antipsychotic sometimes used as an adjunct antidepressant). Other tetracyclic antidepressants that can be conjugated to the dendrimers include, but are not limited to, Benzoctamine (e.g., Tacitin), Loxapine (e.g., Adasuve, Loxitane), Mazindol (e.g., Mazanor, Sanorex), Aptazapine (CGS-7525A; a close analogue of mirtazapine), Esmirtazapine (ORG-50,081; the (S)-(+) enantiomer of mirtazapine), Oxaprotiline (C 49-802 BDA; a close analogue of maprotiline), and Ciclazindol (WY-23,409; a close analogue of mazindol). Benzoctamine (e.g., Tacitin) is a tetracyclic compound and is closely related to maprotiline, with the two compounds differing only in the length of their side chain, but benzoctamine is typically being used as an anxiolytic. Loxapine (e.g., Adasuve, Loxitane) is a typical antipsychotic that produces amoxapine as a major metabolite and is said to have antidepressant effects, but it is not usually regarded as a tetracyclic antidepressant. Drugs that contain four rings not all fused together but could still be classified as tetracyclic include mazindol. Mazindol (Mazanor, Sanorex) is a monoamine reuptake inhibitor generally used as an appetite suppressant and with antidepressant effects.

e. Monoamine oxidase inhibitors (MAOIs)

The compositions may include a dendrimer complexed to one or more monoamine oxidase inhibitors (MAOIs).

Monoamine oxidase inhibitors are responsible for blocking the monoamine oxidase enzyme (Shulman K I, et al., CNS drugs. 2013; 27(10):789-797). The monoamine oxidase enzyme breaks down different types of neurotransmitters from the brain: norepinephrine, serotonin, dopamine, and tyramine. MAOIs inhibit the breakdown of these neurotransmitters thus, increasing their levels and allowing them to continue to influence the cells that have been affected by depression.

There are two types of monoamine oxidase, A and B. The MAO A is mostly distributed in the placenta, gut, and liver, but MAO B is present in the brain, liver, and platelets. The ratio of MAO-A to MAO-B varies throughout the body. In the human brain, the ratio of MAO-A to MAO-B is 25% to 75%, whereas in the liver, the ratio is 50% to 50%. The ratio is 80% to 20% in the intestine, and in the peripheral adrenergic neurons, the ratio is 90% to 10%.

Serotonin and noradrenaline are substrates of MAO A, but phenylethylamine, methylhistamine, and tryptamine are substrates of MAO B. Dopamine and tyramine are metabolized by both MAO A and B. Selegiline and rasagiline are irreversible and selective inhibitors of MAO type B, but safinamide is a reversible and selective MAO B inhibitor.

In some forms, the dendrimer is complexed to one or more reversible and/or irreversible MAOIs. Reversible inhibitors of monoamine oxidase A (RIMAs) are a subclass of MAOIs that selectively and reversibly inhibit the MAO-A enzyme. Suitable reversible MAOIs include but are not limited to befloxatone (MD-370,503), brofaromine (Consonar), furazolidone (Furoxone), Linezolid (Zyvox), Moclobemide (Aurorix), and/or Toloxatone (Humoryl). Some MAOIs covalently bind to the monoamine oxidase enzymes, thus inhibiting them irreversibly; the bound enzyme could not function and thus enzyme activity was blocked until the cell made new enzymes. Suitable irreversible MAOIs which may be conjugated to the dendrimers include but are not limited to clorgyline, iproniazid (marsilid), isocarboxazid (Marplan), Nialamide (Niamid), Pargyline (Eutonyl), Phenelzine (Nardil), Procarbazine (Matulane), Rasagiline (Azilect), Selegiline, 1-deprenyl (Eldepryl and Emsam), and Tranylcypromine (parnate).

f. Benzodiazepines

The compositions may include a dendrimer complexed to one or more Benzodiazepines. “Benzodiazepines” (BZD, BDZ, BZs), are a class of psychoactive drugs whose core chemical structure is the fusion of a benzene ring and a diazepine ring. Benzodiazepines act upon benzodiazepine receptors (BZ-R) in the central nervous system (CNS). The receptor is a protein containing five transmembrane subunits that form a chloride channel in the center, i.e., GABA-A receptor. The five subunits are composed of two alpha, two beta, and one gamma subunit. The extracellular portions of the alpha and beta subunit proteins form a receptor site for gamma-aminobutyric acid (GABA), an inhibitory neurotransmitter. The extracellular portions of the alpha and gamma subunit proteins form a binding site for benzodiazepines. Activation of the BZ-R causes a conformational change to a central pore, which allows the entrance of chloride ions into the neuron. The influx of the chloride anion hyperpolarizes the neuron, resulting in the decreased firing of action potentials of that neuron.

In some forms, the benzodiazepine conjugated to the dendrimer is an ultra-short acting benzodiazepine. Non-limiting examples of ultra-short acting benzodiazepine include but are not limited to flunitrazepam, diazepam, and midazolam. In some forms, the benzodiazepine conjugated to the dendrimer is a short acting benzodiazepine. Non-limiting examples of short acting benzodiazepine include but are not limited to alprazolam, chlordiazepoxide, and lorazepam. In some forms, the benzodiazepine conjugated to the dendrimer is long-acting benzodiazepine. Non-limiting examples of long-acting benzodiazepine include but are not limited to clonazepam, oxazepam, and prazepam.

g. Other Antidepressants

The compositions may include a dendrimer complexed to one or more beta-blockers, neurosteroids, and/or anticonvulsants (Wang M., Neurosteroids and GABA-A Receptor Function. 2011; 2).

“Beta-blockers”, also known as beta-adrenergic blocking agents, block norepinephrine and epinephrine (adrenaline) from binding to beta receptors on nerves. Blocking these transmitters, reduces help control some of the physical symptoms of anxiety. This also helps reduce the heart rate and lower blood pressure. Non-limiting examples of beta-blockers include fluoxetine, paroxetine, duloxetine and bupropion.

“Neurosteroids” as used herein, refers to steroids produced in the brain or transported into the brain that alter neuronal excitability. Suitable neurosteroids include but are not limited to brexanolone (allopregnanolone) and SGE-217, and include GABA modulators such as ganaxolone, minaxolone, and zuranolone.

“Anticonvulsants” as used herein, refers to agents used to prevent, reduce or control seizures of convulsions, includes antidepressants, steroids and other drugs.

### 2. Antipsychotics

a. Typical Antipsychotics

The compositions may include a dendrimer complexed to one or more typical antipsychotics, also known as first generation antipsychotics. The typical antipsychotics generally work by inhibiting dopaminergic neurotransmission; their effectiveness is best when they block activity at D2 dopamine receptors in the brain (Wang Shanghai archives of psychiatry. 2013; 25(3): 134-140). In some forms, the typical antipsychotics may also have noradrenergic, cholinergic, and histaminergic blocking action. The one or more typical antipsychotics conjugated to the dendrimers can belong to one of the following subclasses: phenothiazines, Butyrophenones, thioxanthenes, dihydroindolones, dibenzepines and/or diphenylbutylpiperidines.

i. Phenothiazines

The compositions may include a dendrimer complexed to one or more phenothiazines. The phenothiazines are the largest chemical group of typical antipsychotics, comprising more than 40 compounds (grouped under three subtypes). Phenothiazines share the same three-ring structure with different side chains joined at the nitrogen atom of the middle ring. The activity of the group can be affected by substitutions at position 2 or 10. The phenothiazines are categorized into three subclasses based on substitutions at position 10: aliphatic, piperidine, and piperazine phenothiazines.

In some forms, the one or more phenothiazines conjugated to the dendrimer is an aliphatic (low/medium-potency agents) phenothiazine. Suitable aliphatic phenothiazines include but are not limited to Chlorpromazine, Levomepromazine, Promazine, and Triflupromazine.

In some forms, the one or more phenothiazines conjugated to the dendrimer is a piperidine phenothiazine. Piperidine phenothiazines are low or medium potency agents. Suitable piperidine phenothiazines include but are not limited to Mesoridazine, Pericyazine, Pipotiazine, and Thioridazine.

In some forms, the one or more phenothiazines conjugated to the dendrimer is a piperazine phenothiazine. Piperazine phenothiazines are medium or high potency agents. Suitable piperazine phenothiazines include but are not limited to Perphenazine, Fluphenazine, and Trifluoperazine.

In exemplary embodiments, phenothiazines conjugated to the dendrimer has the formula as shown in Formula X1 below.

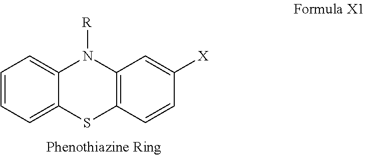

ii. Non-Phenothiazines

The compositions may include a dendrimer complexed to one or more non-phenothiazines. The non-phenothiazines that may be conjugated to the dendrimer may be one or more of butyrophenones, thioxanthenes, dihydroindolones, dibenzepines and/or diphenylbutylpiperidines.

In some forms, the dendrimer is conjugated to one or more butyrophenones and/or diphenylbutylpiperidines. Butyrophenones and diphenylbutylpiperidines are high potency agents. Non-limiting examples of butyrophenones include, but are not limited to, Benperidol, Droperidol, and Haloperidol. Non-limiting examples of diphenylbutylpiperidines include but are not limited to Fluspirilene and Pimozide.

In some forms, the dendrimer is conjugated to one or more thioxanthenes, dibenzepines, and/or dihydroindolones. Thioxanthenes, dibenzepines, and dihydroindolones are low or medium potency agents. Non-limiting examples of thioxanthenes include but are not limited to Clopenthixol, Flupenthixol, Thiothixene, and Zuclopenthixol. Non-limiting examples of dihydroindolones include but are not limited to Molindone. Non-limiting examples of dibenzepines include but are not limited to Clotiapine and Loxapine.

b. Atypical Antipsychotics

The compositions may include a dendrimer complexed to one or more atypical antipsychotics. The term “atypical” refers to an antipsychotic medication that produces minimal extrapyramidal side effects (EPS) at clinically effective antipsychotic doses, has a low propensity to cause tardive dyskinesia (TD) with long-term treatment, and treats both positive and negative signs and symptoms of mental health disorders e.g., schizophrenia. Suitable atypical antipsychotics that may be conjugated to the dendrimer compositions include but are not limited to clozapine (Clozaril), risperidone (Risperdal), olanzapine (Zyprexa), quetiapine (Seroquel), ziprasidone (Geodon), aripiprazole (Abilify), zotepine, cariprazine, brexipiprazole, asenapine (Saphris), iloperidone (Fanapt), amisulpride, blonanserin, melperone, perospirone, remoxipride, sertindole, sulpiride, lurasidone (Latuda), and paliperidone (Invega), the active metabolite of risperidone.

Typically, the dendrimers are conjugated to one or more atypical antipsychotics with high selectivity for serotonin (5-HT) and dopamine (D) receptors. More preferably, the one or more atypical antipsychotics bind as antagonists for 5-HT2A receptors and D2 receptors. For example, the dendrimers are conjugated to one or more of clozapine (Clozaril), risperidone (Risperdal), olanzapine (Zyprexa), quetiapine (Seroquel), ziprasidone (Geodon), aripiprazole (Abilify), zotepine, cariprazine, brexipiprazole, asenapine (Saphris), iloperidone (Fanapt), amisulpride, blonanserin, melperone, perospirone, remoxipride, sertindole, sulpiride, lurasidone (Latuda), and paliperidone (Invega), the active metabolite of risperidone.

The dendrimers can be conjugated to one or more atypical antipsychotics with selectivity for one or more other receptors. For example, some atypical antipsychotics are also potent serotonin-1A (5-HT1A; aripiprazole), serotonin-1C (5-HT1C; clozapine, olanzapine, risperidone), histamine-1 (H1; olanzapine, quetiapine) and α1-(aripiprazole, clozapine, olanzapine, paliperidone, quetiapine) and α2-adrenergic (clozapine, olanzapine, paliperidone, quetiapine, risperidone) receptor blockers. In another example, some atypical antipsychotics, such as clozapine, olanzapine, and quetiapine, bind to histamine-1 (H1) receptors with an affinity comparable to that for D2, 5-HT2A/2C, and α2-adrenoceptors.

In some embodiments, the dendrimers can be conjugated to one or more atypical antipsychotics that has differential effects on the central and nervous system in a tissue-specific manner. For example, risperidone and aripiprazole, despite their inhibitory effect on the firing activity of 5-HT neurons, increases cortical 5-HT levels each on its own and potentiates an escitalopram-induced increase in 5-HT concentrations the frontal cortex. Furthermore, risperidone enhances the anxiolytic and antidepressant-like behavioral effect of escitalopram.

Drugs particularly useful for treatment of ADHD and symptoms thereof can be conjugated to dendrimer for use in treating individuals. These include ADHD drugs such asmethylphenidate, amphetamine, atomoxetine, clonidine, guanfacine, viloxazine, and their analogs/modifications. These drugs are commonly used in patients with depression, PTSD, and bipolar disorder. See https://www.webmd.com/add-adhd/adhd-medication-chart and https://www.additudemag.com/adhd-medication-for-adults-and-children/. In some forms, the efficacy of non-stimulant ADHD drugs may be significantly increased via conjugation to dendrimer.

Some of them have similar mechanisms of action to antidepressants/antipsychotics such as atomoxetine (SNRI), viloxazine (SNRI) and methylphenidate which blocks reuptake of dopamine and norepinephrine. Examples of antidepressants currently used to treat the symptoms of ADHD include bupropion, desipramine, imipramine, and nortriptyline.

### C. Coupling Agents and Spacers

Dendrimer-active agent conjugates can be formed from one or more active agents covalently conjugated or non-covalently attached to a dendrimer. In preferred embodiments, the one or more active agents are covalently conjugated to the dendrimer.

Optionally, the one or more active agents are conjugated to the dendrimer via one or more spacers. The term “spacer” includes chemical moieties and functional groups used for linking an active agent to the dendrimer. The spacer can be either a single chemical entity or two or more chemical entities linked together. The spacer can include any small chemical entity, peptide or polymers having sulfhydryl, thiopyridine, succinimidyl, maleimide, vinylsulfone, carbonate, etc.

In some embodiments, the spacer via which the active agent is conjugated to the dendrimer contains different linkages such as disulfide, ester, carbonate, carbamate, thioester, hydrazine, hydrazides, ether, and amide linkages. The spacer between a dendrimer and an active agent can be designed to provide a releasable or non-releasable form of the dendrimer conjugate in vivo. In some embodiments, the conjugation between active agent and dendrimer is via an appropriate spacer that contains an ester bond between the active agent and the dendrimer. In some embodiments, one or more spacers between a dendrimer and active agents can provide desired and effective release kinetics in vivo. These spacers may contain cleavable linkages (e.g., ester, disulfide, phosphodiester, triglycyl peptide, and hydrazine) or non-cleavable linkages (e.g., amide, ether, and amino alkyl). The conjugation between active agents and dendrimers can be performed using reaction known in the art, such as click chemistry, acid-amine coupling, Steglich esterification, etc.

In some embodiments, the conjugation between active agent and dendrimer is via a spacer that contain disulfide, ester, ether, thioester, carbamate, carbonate, hydrazine, ether, or amide linkages, or a combination thereof. In some embodiments, the conjugation between active agent and dendrimer is via an appropriate spacer that contain an ester linkage or an amide linkage between the agent and the dendrimer depending on the desired release kinetics of the agent.

The spacer can be chosen from among a class of compounds terminating in sulfhydryl, thiopyridine, succinimidyl, maleimide, vinylsulfone and carbonate group. The spacer can include thiopyridine terminated compounds such as dithiodipyridine, N-Succinimidyl 3-(2-pyridyldithio)-propionate (SPDP), Succinimidyl 6-(3-[2-pyridyldithio]-propionamido) hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo (Arg-Gly-Asp-d-Phe-Cys) (c(RGDfC)), cyclo (Arg-Gly-Asp-D-Tyr-Cys), cyclo (Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, 5 mercapto valeric acid and other mercapto derivatives such as 2 mercaptoethanol and 2 mercaptoethylamine. The spacer can be thiosalicylic acid and its derivatives, (4-succinimidyloxycarbonyl-methyl-alpha-2-pyridylthio)toluene, (3-[2-pyridithio]propionyl hydrazide, The spacer can have maleimide terminations wherein the spacer includes polymer or small chemical entity such as bis-maleimido diethylene glycol and bis-maleimido triethylene glycol, Bis-Maleimidoethane, bismaleimidohexane. The spacer can include vinylsulfone such as 1,6-Hexane-bis-vinylsulfone. The spacer can include thioglycosides such as thioglucose. The spacer can be reduced proteins such as bovine serum albumin and human serum albumin, any thiol terminated compound capable of forming disulfide bonds. The spacer can include polyethylene glycol having maleimide, succinimidyl, and/or thiol terminations.

### D. Dendrimer-Agent Conjugates

Dendrimer-active agent conjugates can be formed of antidepressant and/or antipsychotic agents covalently conjugated or non-covalently attached to a dendrimer, a dendritic polymer, or a hyperbranched polymer. Methods for conjugation of one or more active agents to a dendrimer are known, such as those described in U.S. Published 2011/0034422, 2012/0003155, and 2013/0136697.

In some embodiments, one or more active agents are covalently conjugated to one or more terminal groups of the dendrimer such as terminal hydroxyl groups. In some embodiments, dendrimer conjugates include one or more active agents conjugated to the dendrimer via one or more spacers. The spacer between a dendrimer and an active agent can be designed to provide a releasable or non-releasable form of the dendrimer conjugate in vivo. For example, the spacer can be cleavable or contain a chemical linkage that is cleavable, for example, by exposure to the intracellular compartments of target neural and/or glial cells or upon binding to the receptor on the surface or in the interior of the target neural and/or glial cells in vivo. Examples of cleavable linkages that can be used in a spacer of the dendrimer-active agent conjugates include, esterase sensitive ester bond, glutathione sensitive disulfide bond, phosphatase-sensitive phosphodiester bond, oligopeptide such as triglycyl peptide linker capable of lysosomal release, acid cleavable hydrazine linkage etc. In some embodiments, the spacer between a dendrimer and active agents can provide desired and effective release kinetics in vivo. In some embodiments, the spacer between the dendrimer and the active agent can be non-cleavable or contain a chemical linkage that is non-cleavable, such as amide, ether, and amino alkyl linkages.

Generally, the spacer between the dendrimer and active agent has a length sufficient for the active agent conjugated thereto to reach and bind to the target receptor on the surface and/or inside of the target cell. The length of the spacer can vary, depending on the location of the target receptor (for example, on the cell surface, in the cytoplasm of the cell, or in an intercellular compartment of the cell) and/or density of the receptor when located on the cell surface.

The dendrimer can be a generation 2, generation 3, generation 4, generation 5, generation 6, and up to generation 10. In some embodiments, the dendrimer is conjugated to one or more active agents via spacers containing cleavable (ester, disulfide, phosphodiester, triglycyl peptide, and hydrazine) or non-cleavable (amide, ether, and amino alkyl) linkages.

The density of active agents covalently conjugated to or non-covalently attached to the dendrimer can be adjusted based on the specific antidepressant and/or antipsychotic agent being delivered, the target receptors, the target neural and/or glial cells, the location of the target neural and/or glial cells, etc. For example, a plurality of active agents conjugated to the dendrimer are on the periphery of the dendrimer and the surface density of the active agent is at least 1 active agent/nm² (number of active agent conjugated/surface area in nm²). For example, in some embodiments, the surface density of active agent per nm² is more than 2, 3, 4, 5, 6, 7, 8, 9, 10, such as at least 10, 15, 20, 25, 30, 35, 40, 45, 50, or more than 50. In some embodiments, the volumetric density of active agent is between about 4 and about 50 groups/nm³, between about 5 and about 30 groups/nm³, or between about 10 and about 20 groups/nm³.

Typically, the dendrimer-active agent conjugates have a hydrodynamic volume in the nanometer range. For example, in some embodiments, the glucose dendrimer-active agent conjugates including one or more antidepressant and/or antipsychotic agents conjugated to the dendrimer have a diameter of about 2 nm to about 100 nm, or more than 100 nm, up to 500 nm, depending upon the generation of dendrimer, the chemical composition and amount of active agent conjugated thereto. In some embodiments, a dendrimer-active agent conjugate including one or more antidepressant and/or antipsychotic agents conjugated to the dendrimer has a diameter effective to penetrate brain tissue and to retain on the surface and/or in target neural and/or glial cells for a period of time sufficient for the active agent to bind to the targeted receptors on the surface and/or in the target neural and/or glial cells. In some embodiments, a dendrimer-active agent conjugate including one or more antidepressant and/or antipsychotic agents conjugated to the dendrimer has a diameter effective to remain in the peripheral circulation and to retain on the surface and/or in target neural and/or glial cells for a period of time sufficient for the active agent to bind to the targeted receptors on the surface and/or in the target neural and/or glial cells such as for example, neural and/or glial cells of the gastrointestinal system.

The dendrimer-active agent conjugates can be neural, have a positive charge or a negative charge. In some embodiments, the dendrimer-active agent conjugates are neutral. The presence of antidepressant and/or antipsychotic agents derivatives can affect the surface charge of the dendrimer-active agent conjugates. In some embodiments, the surface charge of the dendrimer conjugated to antidepressant and/or antipsychotic agents is between −100 mV and 100 mV, between −50 mV and 50 mV, between −25 mV and 25 mV, between −20 mV and 20 mV, between −10 mV and 10 mV, between −10 m V and 5 mV, between −5 mV and 5 mV, or between −2 mV and 2 mV. The range above is inclusive of all values from −100 mV to 100 mV. In preferred embodiments, the surface charge of the dendrimer-active agent conjugates is neutral or near-neutral, i.e., from about −10 mV to about 10 mV, inclusive.

An exemplary dendrimer-active agent conjugate is represented by Formula (I). The dendrimer of the exemplary conjugate contains surface hydroxyl groups, wherein one or more of the surface hydroxyl groups are conjugate to one or more active agents via one or more spacers as shown in Formula (I) below.

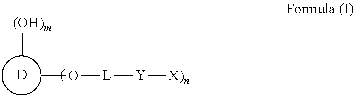

wherein D can be a generation 1 to generation 10 or generation 2 to generation 10 dendrimer, such as any one of those described above, for example, PAMAM (such as hydroxyl-terminated PAMAM dendrimer) or a glucose-based dendrimer; each occurrence of L can be any suitable chemical moiety, preferably containing a triazole moiety; Y can be a bond or a linkage selected from secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, phosphodiester group

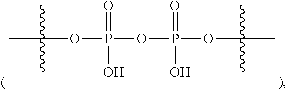 [SMILES: *OP(=O)(O)OP(=O)(O)O*]

hydrazino group, hydrazones, hydrazides, ester (—C(O)—O—), ether (—O—), and oligopeptide (e.g., triglycyl peptide), wherein R is an alkyl group, an aryl group, or a heterocyclic group; each occurrence of X can be an antidepressant and/or antipsychotic agent, wherein a functional group of X (such as an amino group including primary amino, secondary amino, or tertiary amino group; a carboxylic group; or a hydroxyl group) forms a portion of linkage Y; n can be an integer from 1 to 100; and m can be an integer from 16 to 4096.

The oxygen atom shown in Formula (I) is from the surface functional group of the dendrimer, such as a surface hydroxyl group, where the surface hydroxyl group may or may not be part of a terminal sugar moiety/molecule (e.g., glucose). Although not illustrated in Formula (I), one or more hydroxyl groups of the dendrimer that are not conjugated to active agents may be modified with one or more carbohydrates and/or polyalkylene glycols, such as PEG.

When administered to a subject in need thereof, the antidepressant and/or antipsychotic agent X of Formula (I) can bind to a target receptor on the surface of the target cell or inside the target cell. In some embodiments, when the antidepressant and/or antipsychotic agent X binds to the target receptor, the agent X remains conjugated to the dendrimer. In these embodiments, following binding, the agent X may be released from the dendrimer or remain conjugated to the dendrimer as an intact dendrimer-active agent conjugate. In some embodiments, the antidepressant and/or antipsychotic agent X is released from the dendrimer at close proximity to the target receptor and then binds to the target receptor.

In some embodiments, each occurrence of L can be represented by -A′-L1-B′-L2-, wherein A′ can be a carbonyl (—C(O)—) or a bond (including single, double, and triple bonds, for example a single bond); B′ can be a bond (including single, double, and triple bonds, for example a single bond), an amide, an ester, an ether, a thiol, a dithiol, an aryl, a heteroaryl, a polyaryl, a heteropolyaryl, or a heterocyclic; and L1 and L2 can be independently a bond, an alkylene, a heteroalkylene, an aryl, an aralkyl, an ether, a polyether, a thiol, a dithiol, a thiolether, a polythioether, an oligopeptide, a polypeptide, an oligo (alkylene glycol), or a polyalkylene glycol, or L1 and L2 can be independently composed of a combination of these groups, such as a combination of alkylene and polyether, a combination of alkylene and thiol or dithiol, a combination of alkylene and oligopeptide, a combination of alkylene, polyether, and thiol or dithiol, or a combination of polyether and thiol or dithiol. In some forms, L1-B′-L2-together form a chemical moiety selected from an -alkylene-triazole-di(alkylene glycol)-, a -di(alkylene glycol)-triazole-alkylene-, -alkylene-triazole-oligo(alkylene glycol)-, an -oligo(alkylene glycol)-triazole-alkylene-, an -alkylene-triazole-poly(alkylene glycol)-, -poly(alkylene glycol)-triazole-alkylene-, an -alkylene-triazole-ether-, an -alkylene-triazole-alkylene-, an -alkylene-amide-alkylene-, and combinations thereof.

In some embodiments, B′ can be a bond (including single, double, and triple bonds, for example a single bond), an amide group, or a heterocyclic group, such as a triazole group.

In some embodiments, L1 can be a bond; an alkylene, such as a C₁-C₁₀ alkylene, a C₁-C₈ alkylene, a C₁-C₆ alkylene, a C₁-C₈alkylene, a C₁-C₄ alkylene, or a C₁-C₃ alkylene; or an oligo- or poly-(alkylene glycol), such as

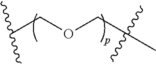

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2

In some embodiments, L2 can be a bond; an alkylene, such as a C₁-C₁₀ alkylene, a C₁-C₈ alkylene, a C₁-C₆ alkylene, a C₁-C₅ alkylene, a C₁-C₄ alkylene, or a C₁-C₃ alkylene; an oligo- or poly-(alkylene glycol), such as

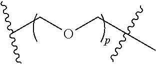

where p is an integer from 1 to 20, from 1 to 18, from 1 to 16, from 1 to 14, from 1 to 12, from 1 to 10, from 1 to 8, from 1 to 6, from 1 to 5, from 1 to 4, from 1 to 3, or 1 or 2; an oligo- or poly-peptide, such as a triglycyl peptide; a thiol; or a dithiol; or L2 is composed of a combination of two or more of alkylene, oligo- or poly-(alkylene glycol), oligo- or poly-peptide, thiols, and dithiols. For example, L2 is represented by

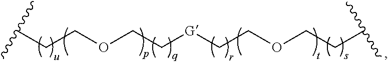

where p, q, r, s, t, and u are independently an integer from 0 to 10, from 0 to 8, from 0 to 6, from 0 to 5, from 0 to 4, from 0 to 3, or from 0 to 2, such as 0, 1, or 2; and G′ is a thiol, a dithiol, an oligo-peptide such as a triglycyl peptide, or a poly-peptide.

In some embodiments, Y is a linkage that is minimally cleavable in vivo. In some embodiments, Y is a linkage that is cleavable in vivo. In some embodiments, Y is an amide (—CONH—), an ester (—C(O)—O—), an ether (—O—), a phosphodiester, or a disulfide group.

In some embodiments, L and Y are both a single bond, and D is directly conjugated to X (an active agent or analog thereof) via an ether linkage.

In some embodiments, D is a generation 2 PAMAM dendrimer, a generation 3 PAMAM dendrimer, a generation 4 PAMAM dendrimer, a generation 5 PAMAM dendrimer, a generation 6 PAMAM dendrimer, a generation 1 glucose dendrimer, a generation 2 glucose dendrimer, a generation 3 glucose dendrimer, a generation 4 glucose dendrimer, a generation 5 glucose dendrimer, or a generation 6 glucose dendrimer.

More specific exemplary dendrimer-active agent conjugates are shown in the Examples below.

### Diagnostic Agents

In some cases, the agents delivered to the target neural and/or glial cells or tissues via dendrimer are diagnostic agents. Examples of diagnostic agents that can be delivered to the brain by glucose dendrimer conjugates include paramagnetic molecules, fluorescent compounds, magnetic molecules, and radionuclides, x-ray imaging agents, and contrast media. Dendrimer conjugates can include agents useful for determining the location of administered compositions. Agents useful for this purpose include fluorescent tags, radionuclides, and contrast agents.

Exemplary diagnostic agents include dyes, fluorescent dyes, near infra-red dyes, SPECT imaging agents, PET imaging agents and radioisotopes. Representative dyes include carbocyanine, indocarbocyanine, oxacarbocyanine, thüicarbocyanine and merocyanine, polymethine, coumarine, rhodamine, xanthene, fluorescein, boron-dipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Exemplary SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-gylcine (MAG3), and hydrazidonicotinamide (HYNIC).

Exemplary isotopes include Tc-94m, Tc-99m, In-111, Ga-67, Ga-68, Gd3+, Y-86, Y-90, Lu-177, Re-186, Re-188, Cu-64, Cu-67, Co-55, Co-57, F-18, Sc-47, Ac-225, Bi-213, Bi-212, Pb-212, Sm-153, Ho-166, and Dy-166.

In exemplary embodiments, the dendrimer compositions include one or more radioisotopes suitable for positron emission tomography (PET) imaging. Exemplary positron-emitting radioisotopes include carbon-11 (¹¹C), copper-64 (⁶⁴Cu), nitrogen-13 (¹³N), oxygen-15 (¹⁵O), gallium-68 (⁶⁸Ga), and fluorine-18 (¹⁸F), e.g., 2-deoxy-2-¹⁸F-fluoro-β-D-glucose (¹⁸F-FDG).

In further embodiments, a singular dendrimer conjugate composition can simultaneously treat and/or diagnose a disease or a condition at one or more locations in the body.

### E. Exemplary Dendrimer-Agent Conjugates

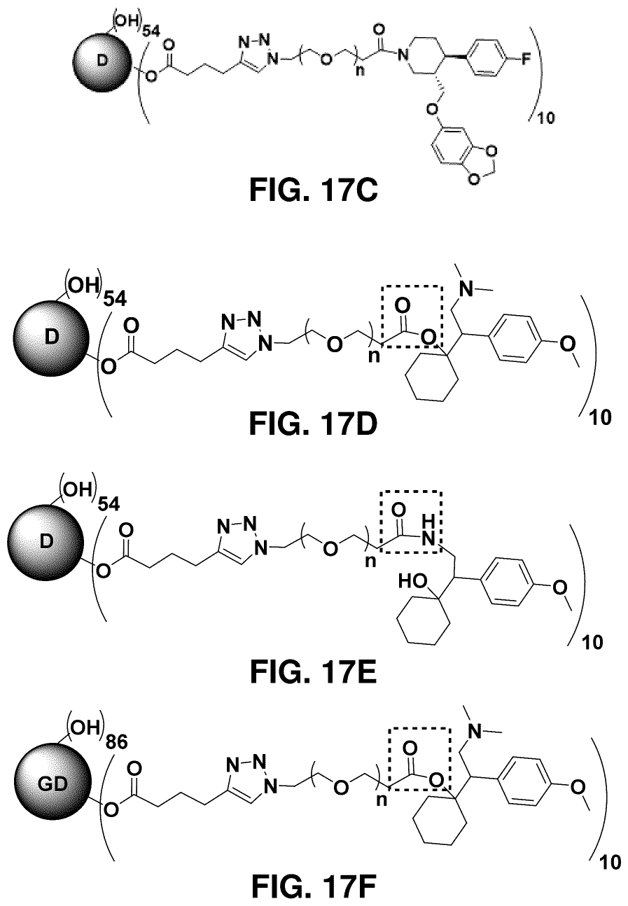

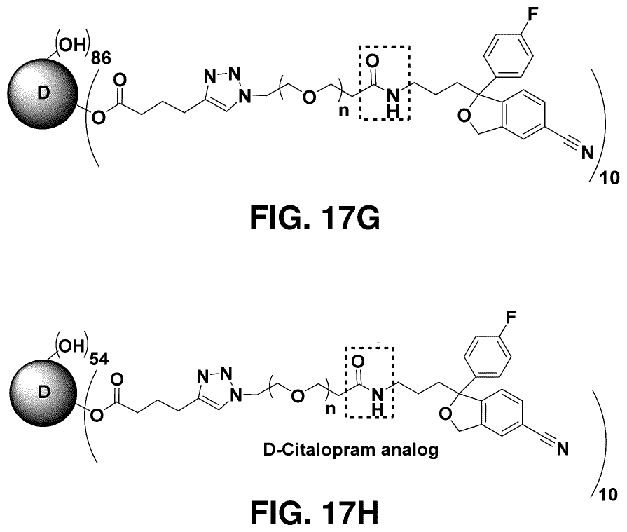

In preferred embodiments, the dendrimer is conjugated to fluoxetine as shown in FIGS. 17A and 17B, wherein n is an integer from 1 to 10, such as 1, 2, 3, 4, 5, 6, 7, 8, 9, or 10.

In other embodiments, the dendrimer is conjugated to paroxetine as shown in FIG. 17C.

In other embodiments, the dendrimer is conjugated to venlafaxine, or a venlafaxine analog as shown in FIGS. 17D-17F, wherein n is an integer from 1 to 10, such as 1, 2, 3, 4, 5, 6, 7, 8, 9, or 10.

In other embodiments, the dendrimer is conjugated to citalopram, or a citalopram analog as shown in FIGS. 17G and 17H, wherein n is an integer from 1 to 10, such as 1, 2, 3, 4, 5, 6, 7, 8, 9, or 10.

### III. Methods of Making Dendrimers and Conjugates Thereof

Methods of synthesizing dendrimers and making dendrimer nanoparticles are also described.

### A. Methods of Making Dendrimers

Dendrimers can be prepared via a variety of chemical reaction steps. Dendrimers are usually synthesized according to methods allowing controlling their structure at every stage of construction. The dendritic structures are mostly synthesized by two main different approaches: divergent or convergent.

In some embodiments, dendrimers are prepared using divergent methods, in which the dendrimer is assembled from a multifunctional core, which is extended outward by a series of reactions, commonly a Michael reaction. The strategy involves the coupling of monomeric molecules that possesses reactive and protective groups with the multifunctional core moiety, which leads to stepwise addition of generations around the core followed by removal of protecting groups. For example, PAMAM-NH₂ dendrimers are first synthesized by coupling N-(2-aminoethyl) acryl amide monomers to an ammonia core.

In other embodiments, dendrimers are prepared using convergent methods, in which dendrimers are built from small molecules that end up at the surface of the sphere, and reactions proceed inward, building inward, and are eventually attached to a core.

Many other synthetic pathways exist for the preparation of dendrimers, such as the orthogonal approach, accelerated approaches, the Double-stage convergent method or the hypercore approach, the hypermonomer method or the branched monomer approach, the Double exponential method; the Orthogonal coupling method or the two-step approach, the two monomers approach, AB₂-CD₂ approach.

In some embodiments, the core of the dendrimer, one or more branching units, one or more spacers, and/or one or more surface functional groups can be modified to allow conjugation to further functional groups (branching units, spacers, surface functional groups, etc.), monomers, and/or agents via click chemistry, employing one or more Copper-Assisted Azide-Alkyne Cycloaddition (CuAAC), Diels-Alder reaction, thiol-ene and thiol-yne reactions, and azide-alkyne reactions (Arseneault M et al., Molecules. 2015 May 20; 20(5):9263-94). In some embodiments, pre-made dendrons are clicked onto high-density hydroxyl polymers. ‘Click chemistry’ involves, for example, the coupling of two different moieties (e.g., a core group and a branching unit; or a branching unit and a surface functional group) via a 1,3-dipolar cycloaddition reaction between an alkyne moiety (or equivalent thereof) on the surface of the first moiety and an azide moiety (e.g., present on a triazine composition or equivalent thereof), or any active end group such as, for example, a primary amine end group, a hydroxyl end group, a carboxylic acid end group, a thiol end group, etc.) on the second moiety. In some embodiments, one or more hydroxyl groups on the surface of the dendrimer (hydroxyl-terminated PAMAM dendrimer or glucose dendrimer) are modified to contain an alkyl group and a drug is modified to contain an azide group. Alternatively, one or more hydroxyl groups on the surface of the dendrimer (hydroxyl-terminated PAMAM dendrimer or glucose dendrimer) are modified to contain an azide group and a drug is modified to contain an alkyne group. The azide and alkyne are then reacted via a 1,3-dipolor cycloaddition reaction to form a triazole moiety.

In some embodiments, dendrimer synthesis relies upon one or more reactions such as thiol-ene click reactions, thiol-yne click reactions, CuAAC, Diels-Alder click reactions, azide-alkyne click reactions, Michael Addition, epoxy opening, esterification, silane chemistry, and a combination thereof.

In some embodiments, methods involve one or more protection and deprotection steps of the function groups (e.g., hydroxyl groups) on the central core, branching units, and/or therapeutic, prophylactic or diagnostic agents to facilitate addition of branching units to generate desired dendrimer molecules, or addition of therapeutic, prophylactic or diagnostic agents to generate desired dendrimer conjugates. In the case of hydroxyl groups, they may be protected by formation of an ether, an ester, or an acetal. Other exemplary protection groups include Boc and Fmoc.

Any existing dendritic platforms can be used to make dendrimers of desired functionalities, i.e., with a high-density of surface hydroxyl groups by conjugating high-hydroxyl containing moieties such as 1-thio-glycerol or pentaerythritol. Exemplary dendritic platforms such as polyamidoamine (PAMAM), poly (propylene imine) (PPI), poly-L-lysine, melamine, poly (etherhydroxylamine) (PEHAM), poly (esteramine) (PEA) and polyglycerol can be synthesized and explored.

Dendrimers also can be prepared by combining two or more dendrons. Dendrons are wedge-shaped sections of dendrimers with reactive focal point functional groups. Many dendron scaffolds are commercially available. They come in 1, 2, 3, 4, 5, and 6th generations with, respectively, 2, 4, 8, 16, 32, and 64 reactive groups. In certain embodiments, one type of agents is linked to one type of dendron and a different type of agent is linked to another type of dendron. The two dendrons are then connected to form a dendrimer. The two dendrons can be linked via click chemistry i.e., a 1,3-dipolar cycloaddition reaction between an azide moiety on one dendron and alkyne moiety on another to form a triazole linker.

Exemplary methods of making dendrimers are described in detail in International Patent Publication Nos. WO2009/046446, WO2015168347, WO2016025745, WO2016025741, WO2019094952, and U.S. Pat. No. 8,889,101.

### 1. Methods of Making Glucose Dendrimers

In some embodiments, glucose-based dendrimers are prepared using divergent methods, in which the dendrimer is assembled from a multifunctional core, which is extended outward by a series of reactions. The strategy involves the coupling of monomeric molecules that possesses reactive and protective groups with the multifunctional core moiety, which leads to stepwise addition of generations around the core followed by removal of protecting groups.

In some embodiments, glucose dendrimers are synthesized by coupling AB₄ peracetylated β-D glucose-PEG4-azide monomers to hexapropargylated core. In preferred embodiments, the hypercore is prepared from dipentaerythritol, for example by performing propargylation of dipentaerythritol to achieve the hexa-propargylated core. An exemplary scheme for preparing such a glucose dendrimer is shown by Scheme I.

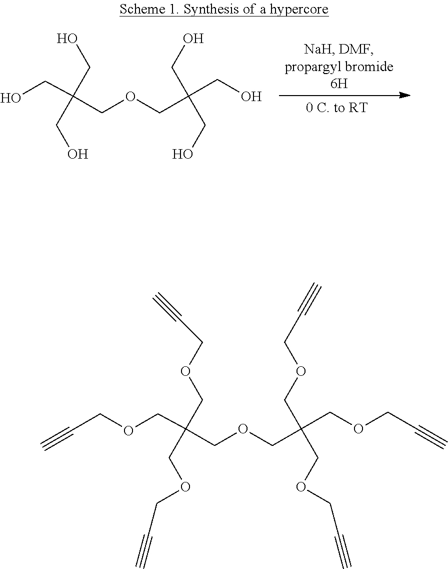 [SMILES: OCC(CO)(CO)COCC(CO)(CO)CO ; C#CCOCC(COCC#C)(COCC#C)COCC(COCC#C)(COCC#C)COCC#C]

In some embodiments, the branching units are hypermonomers i.e., ABₙ building blocks. Exemplary hypermonomers include AB₃, AB₄, AB₅, AB₆, AB₇, AB₈ building blocks. Hypermonomer strategy drastically increases the number of available end groups. An exemplary hypermonomer is AB₄ orthogonal hypermonomer including one azide functional group and four allyl groups prepared from dipentaerythritol with five allyl groups reacted with mono tosylated triethylene glycol azide.

In some embodiments, the branching unit is polyethylene glycerol linear or branched e.g. as shown by Formula III. Other monomers include disaccharides and oligosaccharides, as well as saccharides such as fructose, lactose, and sucrose.

a. Synthesis of AB₄ Building Block

Some exemplary synthesis methods of hypermonomer AB₄ are described below. In some embodiments, the hypermonomer AB₄ is based on glucose molecules. In preferred embodiments, the hypermonomer AB₄ is conjugated to a polyethylene glycerol, for example, tetraethylene glycol (PEG4). In one embodiment, the hypermonomer AB₄ is peracetylated β-D-Glucopyranoside tetraethylene glycol azide.

In some embodiments, the synthesis of glucose-OAc-TEG-OTs involves the following steps: a solution of peracetylated β-D-glucopyranoside (10 g, 25.6 mmol) was dissolved in 50 mL of anhydrous dichloromethane (DCM) followed by addition of 2-(2-(2-(2-hydroxyethoxy) ethoxy) ethoxy)ethyl 4-methylbenzenesulfonate (6.2 g, 17.9 mmol) and the reaction mixture was cooled to 0° C. Boron trifluoride diethyl etherate (2.5 eq.) was added and the reaction was allowed to come to room temperature. The reaction was monitored with the help of TLC and quenched after 5 hrs by the addition of saturated sodium bicarbonate solution at 0° C. After 10 minutes of stirring, DCM (300 mL) was added and the organic layer was washed with saturated sodium bicarbonate solution 3 times until the effervescence was quenched. The reaction mixture was dried over sodium sulfate, filtered, and evaporated under reduced pressure. The crude product was purified by combiflash chromatography using ethyl acetate/hexanes (70:30) mixture as eluents. The desired compound was achieved in 60% yield. Structure of glucose-OAc-TEG-OTs is shown below:

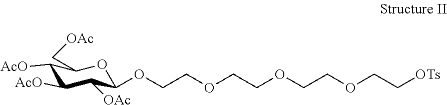

In some embodiments, the synthesis of glucose-OAc-TEG-N₃ involves the following steps: a solution of glucose-OAc-TEG-OTs (6 g, 8.8 mmoles) is dissolved in 40 mL of anhydrous DMF followed by the addition of sodium azide (2 eq) and the reaction mixture is heated to 50° C. for overnight. Upon completion, the reaction mixture is filtered and DMF is evaporated. Once dried, the crude reaction mixture is passed through combiflash using ethyl acetate:hexane (70:30) as eluent. Structure of glucose-OAc-TEG-N₃ is shown below:

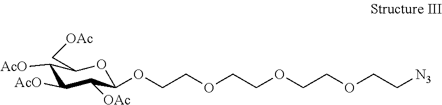

In some embodiments, the synthesis of glucose-OH-TEG-N₃ involves the following steps: the peracetylated β-D-Glucopyranoside tetraethylene glycol azide is dissolved in anhydrous methanol and sodium methoxide is added to adjust the pH around 8.5-9. The reaction is stirred overnight at room temperature, then diluted with methanol and pH is adjusted with Amberlist IR-120+ around 6-7. The reaction mixture is separated by filtration and the solvent removed by rotary evaporation. Structure of glucose-OH-TEG-N₃ is shown below.

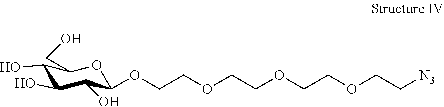

b. Synthesis of Glucose Dendrimers

In some embodiments, glucose dendrimers are synthesized by coupling AB₄ peracetylated β-D glucose-PEG4-azide monomers to hexapropargylated core. In preferred embodiments, the hexapropargylated core is linked to AB₄ β-D-glucose-PEG4-azide building block (2) via click reaction to obtain generation 1 dendrimer.

In some embodiments, generation one dendrimer D1-Glu6-OAc24 is prepared according to the following: Hexapropargylated compound (0.5 g, 1 mmoles) and an azido derivative ((4.1 g, 7.4 mmoles) 1.2 eq. per acetylene) are suspended in a 1:1 mixture of DMF and water in a 20 mL microwave vial equipped with a magnetic stir bar. CuSO₄·5H₂O (5 mol %/acetylene, 75 mg) and sodium ascorbate (5 mol %/acetylene, 60 mg) dissolved in the minimum amount of water are added. The reaction is irradiated in a microwave at 50° C. for 6 h. The reaction mixture is dialyzed against DMF followed by water dialysis containing EDTA. The EDTA is further removed by extensive water dialysis. The product is lyophilized to obtain D1-Glu6-OAc24. Structure of D1-Glu6-OAc24 is shown below.

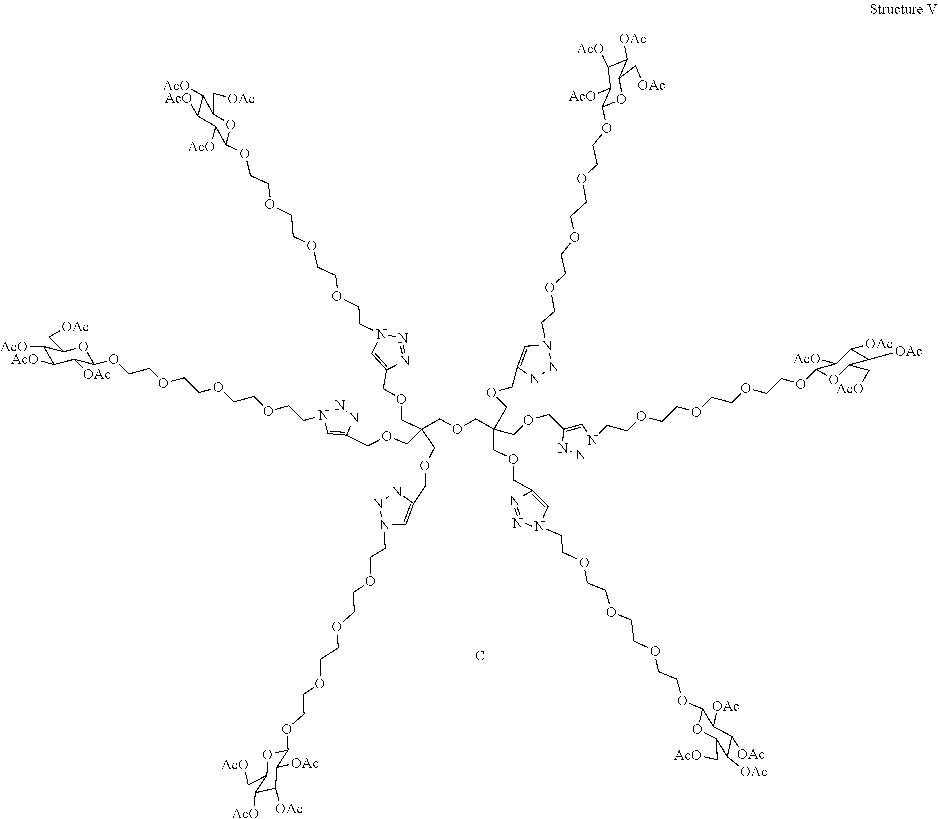

In some embodiments, generation one dendrimer D1-Glu₆-OH₂₄ is prepared according to the following: the peracetylated generation 1 glucose dendrimer (1 g, 0.26 mmoles) is dissolved in anhydrous methanol and sodium methoxide is added to adjust the pH to around 8.5-9. The reaction is stirred overnight at room temperature, then diluted with methanol and pH is adjusted with AMBERLIST® IR-120+ around 6-7. The reaction mixture is separated by filtration and the solvent removed by rotary evaporation, followed by water dialysis. Structure of generation one glucose dendrimer, D1-Glu6-OH24, is shown below.

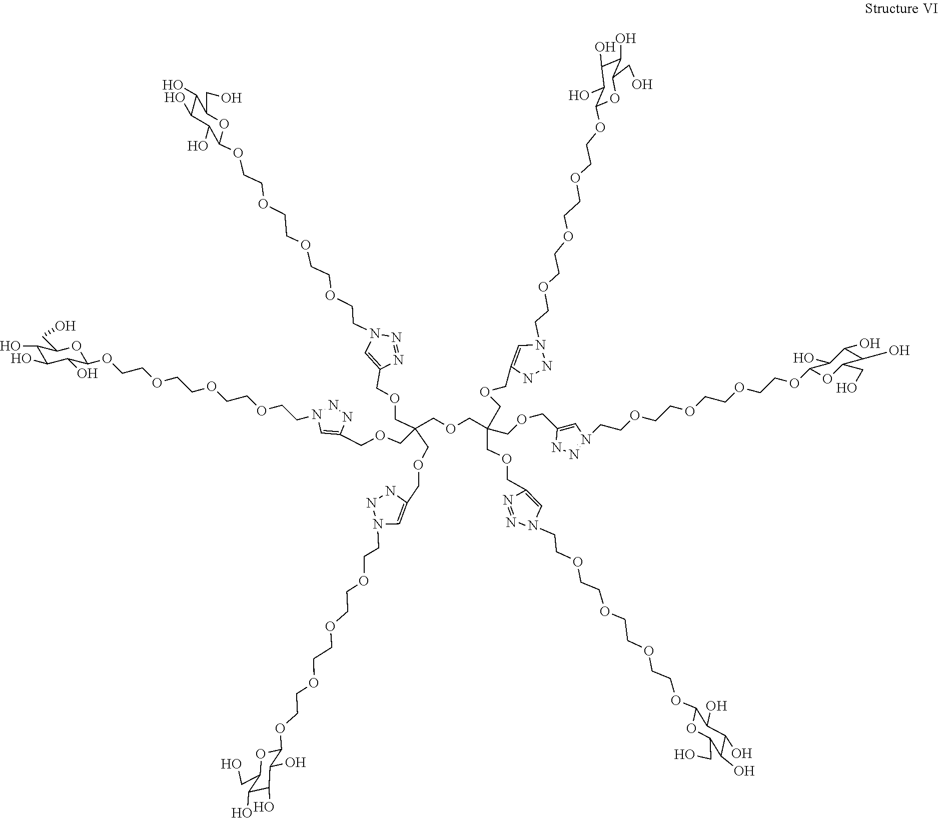

In some embodiments, generation one glucose dendrimer D1-Glu6-OH24 is propargylated to provide D1-Acetylene24 according to the following: D1-GLu6-OH24 (2 g, 0.721 mmol) was dissolved in anhydrous dimethylformamide (DMF, 50 mL) by sonication. Sodium hydride [60% dispersion in mineral oil] (951 mg, 39.65 mmol) is slowly added in portions at 0° C. to the solution with stirring. The solution is stirred for an addition 15 minutes at 0° C. This is followed by the addition of propargyl bromide (3.85 mL, 34.608 mmol, 80% w/w solution in toluene) at 0° C. and the stirring is continued at room temperature for another 6 h. The reaction mixture is quenched with ice and water, filtered, and dialyzed against DMF, followed by the water dialysis to afford D1-acetylene24. Structure of D1-acetylene24 is shown below.

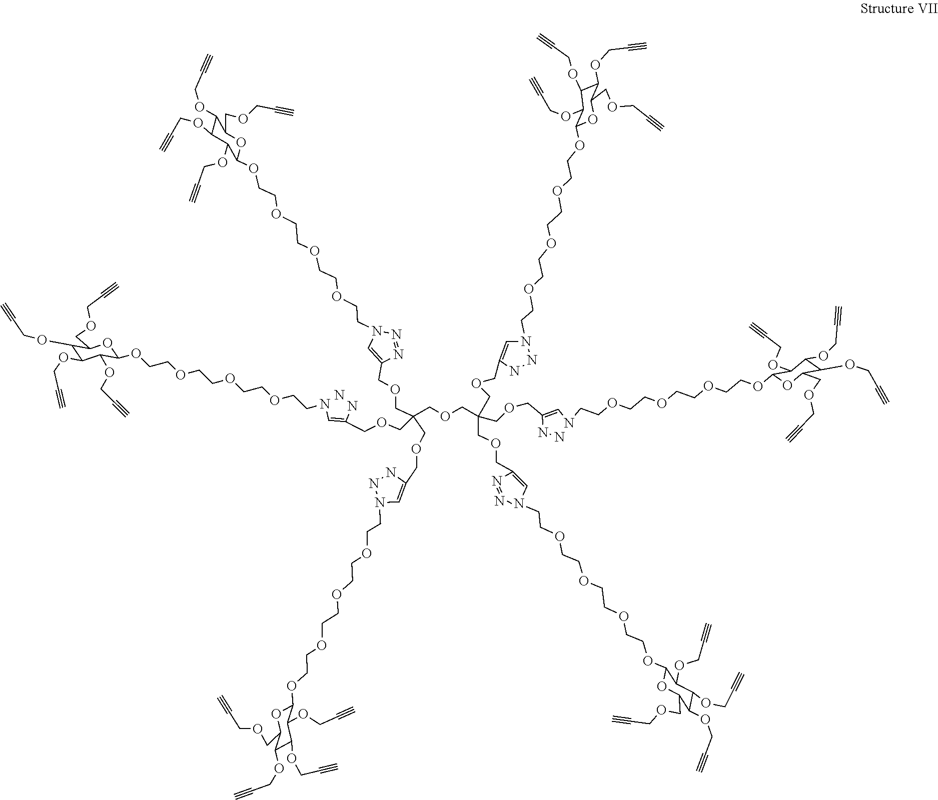

In some embodiments, generation one dendrimer D1-acetylene24 is further reacted with AB₄ β-D-glucose-PEG4-azide to provide generation 2 dendrimer with 24 glucose molecules containing 96 surface hydroxyl groups.

An exemplary generation two dendrimer D2-Glu24-OAc96 is prepared according to the following: D1-acetylene dendrimer24 (0.5 g, 0.13 mmoles) and glucose-OAc-TEG-azide (2.2 g, 4 mmoles) are suspended in a 1:1 mixture of DMF and water in a 20 mL microwave vial equipped with a magnetic stir bar. To this CuSO₄·5H₂O (5 mol %/acetylene, 5 mg) and sodium ascorbate (5 mol %/acetylene, 10 mg) dissolved in the minimum amount of water are added. The reaction is irradiated in a microwave at 50° C. for 8 h. Upon completion, the reaction mixture is dialyzed against DMF followed by water dialysis containing EDTA. The EDTA is further removed by extensive water dialysis. The product is lyophilized to obtain D2-Glu24-OAc96.

In some embodiments, generation two dendrimer D2-Glu24-OH96 is prepared according to the following: the peracetylated generation 2 glucose dendrimer D2-Glu24-OH96 is dissolved in anhydrous methanol and sodium methoxide is added to adjust the pH around 8.5-9.0. The reaction is stirred overnight at room temperature, then diluted with methanol and pH is adjusted with AMBERLIST® IR-120+ around 6-7. The reaction mixture is filtered to remove the resin and the filtrate is evaporated by rotary evaporation followed by water dialysis to obtain the product as off-white solid.

Structure of generation two glucose dendrimer, D2-Glu24-OH96, is shown below.

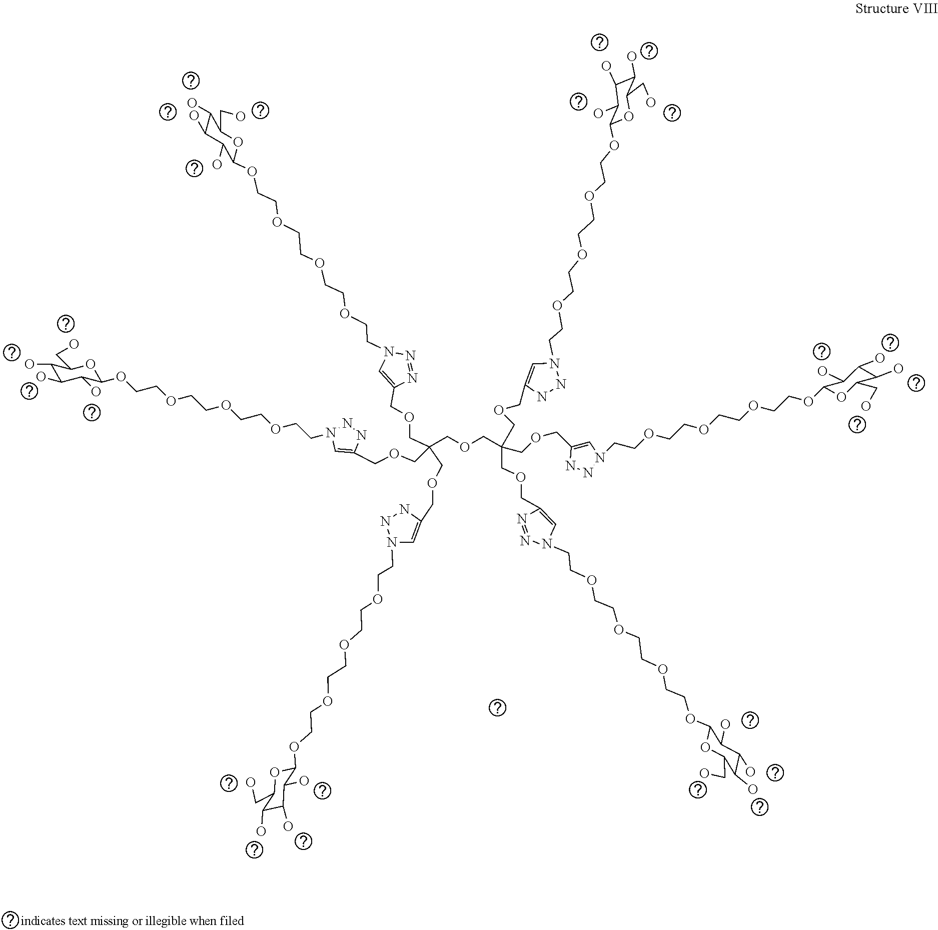

In some embodiments, generation two dendrimer D2-Glu24-OH96 is propargylated at one or more terminal hydroxyl groups suitable for further conjugation to one or more therapeutic, prophylactic or diagnostic agents. In some embodiments, one or more terminal hydroxyl groups of generation two dendrimer D2-Glu24-OH96 is propargylated according to the following: D2-Glu24-OH96 (5b) (200 mg, 0.016 mmol) is dissolved in anhydrous dimethylformamide (DMF, 10 mL) by sonication. To this stirring solution, sodium hydride [60% dispersion in mineral oil] (22 mg, 0.934 mmol) is slowly added in portions at 0° C. The solution is additionally stirred for 15 minutes at 0° C. This is followed by the addition of propargyl bromide (18.0 μL, 80% w/w solution in toluene) at 0° C. and the stirring is continued at room temperature for another 6 h. The solvent is evaporated using V10 evaporator system and the crude product is purified by passing through PD10 SEPHADEX® G25 M column. The aqueous solution is lyophilized to afford the product as off-white solid.

In some embodiments, one or more fluorescent dyes such as infrared fluorescent Cy5 dyes are conjugated to generation two dendrimer D2-Glu24-OH96. In one embodiment, Cy5-D2-Glu24-OH96 (compound 7 of FIG. 1B) is prepared according to the following: Compound 6 (200 mg, 0.016 mmol) and Cy5 azide (20.7 mg, 0.02 mmol) are suspended in a 1:1 mixture of DMF and water in a 25 mL round bottom flask equipped with a magnetic stir bar. To this, CuSO₄·5H₂O (5 mol %/acetylene, 0.3 mg) and sodium ascorbate (10 mol %/acetylene, 0.5 mg) dissolved in the minimum amount of water are added. The reaction is stirred at room temperature for 24 h. Upon completion, the DMF is evaporated using V10 and the purification is performed using PD10 Sephadex G25 M column. The aqueous solution is lyophilized to afford the product as blue solid.

In some embodiments, the total hydroxyl groups for further conjugation to active agents including therapeutic and/or diagnostic agents are about 1-30, 2-20, or 5-10 out of total 96 available hydroxyl groups of the exemplary generation 2 dendrimer with 24 glucose molecules containing 96 surface hydroxyl groups.

### B. Methods of Making Dendrimer-Agent Conjugates

Methods for conjugating agents with dendrimers are generally known in the art and for example, as described in U.S. Published Application Nos. US 2011/0034422, US 2012/0003155, and US 2013/0136697.

In some embodiments, one or more agents are covalently attached to the dendrimers. In some embodiments, the agents are attached to the dendrimer via a spacer that is designed to be non-cleavable in vivo. In some embodiments, the agents are attached to the dendrimer via a spacer that is designed to be cleaved in vivo. For example, the spacer can be designed to be cleaved hydrolytically, enzymatically, or combinations thereof, so as to provide for the sustained release of the agents in vivo. In some embodiments, both the chemical structure of the spacer and its point of attachment to the agent, can be selected so that cleavage of the spacer releases either an agent, or a suitable prodrug thereof. The chemical structure of the spacer can also be selected in view of the desired release rate of the agents.

In some embodiments, the conjugation between the agent and dendrimer is via one or more of disulfide, ester, ether, phosphodiester, triglycyl peptide, hydrazine, amide, or amino alkyl linkages. In some embodiments, the conjugation between the agent and dendrimer is via an appropriate spacer that provides an ester bond or an amide bond between the agent and the dendrimer depending on the desired release kinetics of the agent. In some cases, an ester or disulfide bond is introduced for releasable form of agents. In other cases, an amide or amino alkyl bond is introduced for non-releasable form of agents.

Spacers generally contain one or more organic functional groups. Examples of suitable organic functional groups contained in the spacers include secondary amides (—CONH—), tertiary amides (—CONR—), sulfonamide (—S(O)₂—NR—), secondary carbamates (—OCONH—; —NHCOO—), tertiary carbamates (—OCONR—; —NRCOO—), carbonate (—O—C(O)—O—), ureas (—NHCONH—; —NRCONH—; —NHCONR—, —NRCONR—), carbinols (—CHOH—, —CROH—), disulfide groups, hydrazones, hydrazides, ethers (—O—), and esters (—COO—, —CH₂O₂C—, CHRO₂C—), wherein R is an alkyl group, an aryl group, or a heterocyclic group. In general, the identity of the one or more organic functional groups within the spacer is chosen in view of the desired release rate of the agents. In addition, the one or more organic functional groups can be selected to facilitate the covalent conjugation of the agents to the dendrimers. In some embodiments, the conjugation between the agent and dendrimer is via an appropriate spacer that provides a disulfide bridge between the agent and the dendrimer. In some embodiments, the dendrimer-active agent conjugates are capable of rapid release of the agent in vivo by thiol exchange reactions, under the reduced conditions found in body.

In certain embodiments, the spacer contains one or more of the organic functional groups described above in combination with a linking group. The linking group can be composed of any assembly of atoms, including oligomeric and polymeric chains; for example, the total number of atoms in the linking group is between 3 and 200 atoms, between 3 and 150 atoms, between 3 and 100 atoms, or between 3 and 50 atoms. Examples of suitable linking groups include alkyl groups, heteroalkyl groups, alkylaryl groups, oligo- and polyethylene glycol chains, and oligo- and poly (amino acid) chains. Variation of the linking group provides additional control over the release of the agents in vivo. In embodiments where the spacer includes a linking group, one or more organic functional groups will generally be used to connect the linking group to both the anti-inflammatory agent and the dendrimers.

Reactions and strategies useful for the covalent conjugation of agents to dendrimers are known in the art. See, for example, March, “Advanced Organic Chemistry,” 5th Edition, 2001, Wiley-Interscience Publication, New York and Hermanson, “Bioconjugate Techniques,” 1996, Elsevier Academic Press, U.S.A. Appropriate methods for the covalent conjugation of a given agent can be selected in view of the linking moiety desired, as well as the structure of the agents and dendrimers as it relates to compatibility of functional groups, protecting group strategies, and the presence of labile bonds.

The amount of active agent in the dendrimer-active agent conjugates (drug loading) depends on many factors, including the choice of active agent, dendrimer structure and size, and tissues to be treated. In some embodiments, the one or more antidepressant and/or antipsychotic agents are conjugated to the dendrimer at a concentration between about 0.01% and about 45%, inclusive; between about 0.1% and about 30%, inclusive; between about 0.1% and about 20%, inclusive; between about 0.1% and about 10%, inclusive; between about 1% and about 10%, inclusive; between about 1% and about 5%, inclusive; between about 3% and about 20% by weight, inclusive; or between about 3% and about 10% by weight, inclusive. However, specific drug loading for any given active agent, dendrimer, and site of target can be identified by routine methods, such as those described.

In some embodiments, the conjugation of agents/spacers occurs via about 1%, 2%, 3%, 4%, or 5% of the total available surface functional groups, such as hydroxyl groups, of the dendrimers prior to the conjugation. In other embodiments, the conjugation of agents/spacers occurs on less than 5%, less than 10%, less than 15%, less than 20%, less than 25%, less than 30%, less than 35%, less than 40%, less than 45%, less than 50%, less than 55%, less than 60%, less than 65%, less than 70%, less than 75% total available surface functional groups of the dendrimers prior to the conjugation with active agents. In some embodiments, dendrimer-active agent conjugates retain an effective amount of surface functional groups for targeting to target neural and/or glial cells, whilst conjugated to an effective amount of agents for treat, prevent, and/or image the disease or disorder. In some embodiments, dendrimer-active agent conjugates retain an effective amount of active agents for targeting to target neural and/or glial cells and binding to target receptors on the surface or in the interior of the target neural and/or glial cells.

More specific methods for preparing exemplary dendrimer-active agent conjugates are described in the Examples below.

### IV. Pharmaceutical Formulations

Pharmaceutical compositions including dendrimer-active agent conjugates may be formulated in a conventional manner using one or more physiologically acceptable carriers, optionally including excipients and auxiliaries which facilitate processing of the active compounds into preparations which can be used pharmaceutically, for oral, mucosal (intranasal, buccal, sublingual, vaginal, rectal or pulmonary), transdermal, or injection (intravenous, subcutaneous, intraperitoneal, intramuscular, or intrathecal administration).

Representative excipients include aqueous buffers, wetting agents, viscosity modifiers, tonicity agents, stabilizing agents, and combinations thereof. Suitable pharmaceutically acceptable excipients are preferably selected from materials which are generally recognized as safe (GRAS) and may be administered to an individual without causing undesirable biological side effects or unwanted interactions.

Generally, pharmaceutically acceptable salts of the actives can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs, or extrapolated from human data. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

In certain embodiments, the compositions are administered locally, for example, by injection directly into a site to be treated or via an implant or pump. In some embodiments, the compositions are injected, topically applied, or otherwise administered directly into the vasculature onto vascular tissue at or adjacent to a site of injury, surgery, or implantation. For example, in embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous, or subcutaneous injection) and enteral routes of administration are described. These include administration to mucosal surfaces (nasal, buccal, sublingual, pulmonary, vaginal and rectal).

### A. Parenteral Administration

The compositions of dendrimer-active agent conjugates can be administered parenterally. The phrases “parenteral administration” and “administered parenterally” are art-recognized terms and include modes of administration other than enteral and topical administration. The dendrimers can be administered orally, intranasally, subcutaneously, intraperitoneally, intravenously, intrathecally, or intramuscularly. For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's ‘dextrose. In general, water, saline, aqueous dextrose and related sugar solutions, and glycols such as propylene glycols or polyethylene glycols are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions of dendrimer-active agent conjugates can be administered enterally (orally, sublingually, vaginally, rectally, buccally, intranasally, pulmonarily, or transdermally). The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides, and other components of infant formulas.

Oral formulations may be in the form of chewing gum, gel strips, tablets, capsules, or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

Formulations for administration to mucosal surfaces such as the nose, buccal surfaces or pulmonary, typically contain pharmaceutically acceptable excipients such as those used for parenteral administration, alone or in combination with various surfactants, penetration enhancers, etc.

The compositions can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, dendrimer conjugation may increase the effectiveness and durability of the treatment, which may reduce the need for repeated administration to once per week, once per month, once per six months, once per year, or other longer-term dosing regimens. Some embodiments may be incorporated into drug delivery systems (e.g., implants, pumps, patches, creams, etc.) in order to provide controlled, sustained delivery in a manner that reduces the need for compliance and the potential for abuse.

### V. Methods of Use

In preferred embodiments, the dendrimer compositions traverse the barrier interfaces of the central and peripheral nervous system, and selectively target specific cells and specific receptors on the cells to address a variety of diseases, disorders, injuries, and conditions. The methods include administering to a subject in need, the compositions in an amount effective to increase permeability of the antidepressant and/or antipsychotic agent across the barrier interfaces of the central and peripheral nervous system, and/or increase binding of the antidepressant and/or antipsychotic agents at specific receptors in specific cells, particularly the serotonergic receptors, noradrenergic receptors, adrenergic receptors, and dopaminergic receptors in the central nervous system, peripheral nervous system, and/or cells in peripheral circulation, e.g., neural cells, glial cells, cardiovascular cells, gastrointestinal cells, immune cells.

### A. Methods of Treatment

The compositions can be administered to treat, prevent, or manage the symptoms of a variety of disorders, diseases, and conditions including but not limited to anxiety disorders, mood disorders, eating disorders, personality disorders, stress disorders, and/or psychotic disorders, as well as ADHD and individuals with symptoms of ADHD. In some forms, when the dendrimer is complexed to one or more antidepressants, the compositions can be administered to treat one or more neurological disorders such as mental health disorders e.g., mood disorders, anxiety disorders, eating disorders, substance-related disorders, and post-traumatic disorders; degenerative disorders e.g., Parkinson's disease and Alzheimer's disease; pain disorders e.g., neuropathic pain, gastrointestinal disorders, and/or cardiovascular disorders. In some forms, when the dendrimer is complexed to one or more antipsychotics, the compositions can be administered to treat psychoses that occurs in bipolar disorder, schizophrenia, and/or degenerative disorders. In other forms, the dendrimer-antipsychotic conjugates may be administered to a subject in need for stabilizing moods e.g., in bipolar disorder, reducing anxiety in anxiety disorders and reducing tics in Tourette syndrome. In yet other forms, the dendrimer is complexed to one or more antidepressants and/or antipsychotics to treat non-neural diseases such as gastrointestinal and cardiovascular diseases.

Typically, an effective amount of dendrimer complexes including a combination of a dendrimer with one or more therapeutic, prophylactic, and/or diagnostic active agents are administered to an individual in need thereof. The dendrimers may also include a targeting agent, but as demonstrated by the examples, these are not required for delivery to injured tissue in the spinal cord, the brain, and related areas.

In some embodiments, the dendrimer complexes include an agent that is attached or conjugated to dendrimers, which are capable of preferentially releasing the drug at the target receptor. The agent can be either covalently attached or intra-molecularly dispersed or encapsulated. The amount of dendrimer complexes administered to the subject is selected to deliver an effective amount to reduce, prevent, or otherwise alleviate one or more clinical or molecular symptoms of the disease or disorder to be treated compared to a control, for example, a subject treated with the active agent without dendrimer.

### 1. Site-Specific Targeting

The compositions and methods are designed to circumvent existing challenges in selective drug delivery to the central and peripheral nervous system. The compositions and methods may increase drug bioavailability in the central and peripheral nervous system by one or more of the following: (i) increasing drug density across brain barriers, particularly the blood-brain and blood-cerebrospinal fluid barriers, (ii) increasing drug solubility, (iii) facilitating target engagement i.e., increasing site-specific binding, (iv) improving drug pharmacokinetics, and (v) improving intracerebral distribution. For example, in some forms, the compositions and methods permit selective delivery of compounds to the peripheral nervous system, thereby increasing the potential of the compositions to be used to selectively treat periphery-specific diseases and disorders, including but not limited to neuropathic pain, regional anesthesia, traumatic nerve injury, and inherited and inflammatory neuropathies.

a. Improve Drug Permeability Across Barrier Interfaces

The dendrimer compositions and methods may improve the delivery of the antidepressant and/or antipsychotic agents across one or more of the barrier interfaces in the brain and nervous system, particularly the Blood Brain Barrier (BBB), the CSF-blood barrier, and the blood nerve barrier. These barrier interfaces typically protect neurons from blood-borne substances and help maintain water homeostasis and appropriate milieu for neuronal function in the blood. Due to the clinical significance of antidepressant and antipsychotic agents, the dendrimer compositions may be used to deliver antidepressant and/or antipsychotic agents with improved permeability across these barrier interfaces for site-specific targeting.

i. The Blood Brain Barrier

The “Blood Brain Barrier” (BBB) is a continuous endothelial membrane that, along with pericytes and other components of the neurovascular unit, limits the entry of toxins, pathogens, and blood cells to the brain. However, the BBB also represents an obstacle in the delivery of drugs to the central nervous system (CNS), in part because (1) delivering drugs intended for the brain via systemic routes may result in unacceptably high levels of drugs in the periphery; (2) the complex interplay of cells and molecules that contribute to the BBB's structure and function makes it challenging to determine drug permeability at the BBB, drug distribution in the brain, and target engagement in the brain.

Brain microvascular endothelial cells, pericytes, astrocytes, tight junctions, neurons, and basal membrane construct physically tight brain capillaries in the BBB. The brain capillary endothelial cells do not have fenestrations, which limits the diffusion of small molecules and proteins. Inter-endothelial junctions link the endothelial cells to a continuous barrier, severely restricting the penetration of water-soluble substances. Pericytes, astrocytes and basal membrane surround the endothelial cells and finally form the impermeable BBB. Additionally, efflux transporters are located in brain capillary endothelial cells, which are further obstacles against substances entering the brain. The permeability of the BBB is mainly controlled by inter-endothelial junctions that are protein complexes such as adherens junctions, tight junctions, and gap junctions. Adherens junctions primarily regulate the permeability of the endothelial barrier. Tight junctions play a vital role in sustaining the permeability barrier of epithelial and endothelial cells, which control tissue homeostasis. Gap junctions, composed of six connexin molecules, direct electric, and chemical communication between endothelial cells. Finally, instead of having a static structure, the components of the BBB continuously adapt in response to various physiological changes in the brain. The dendrimer compositions and methods of the present application overcome the aforementioned challenges and are suitable for delivering antidepressant and/or antipsychotic agents across the blood-brain barrier via one or more of the above-described transport mechanisms.

Molecules cross the BBB by a paracellular pathway (between adjacent cells) or a transcellular pathway (through the cells). For the paracellular pathway, ions and solutes utilize concentration gradients to pass the BBB by passive diffusion. The transcellular pathway includes different mechanisms such as passive diffusion, receptor-mediated transport, and transcytosis.

The physicochemical factors that influence BBB permeability include molecular weight, charge, lipid solubility, surface activity and relative size of the molecule. BBB permeability can also be influenced by physiological factors such as efflux transporters, e.g., P-glycoprotein (P-gp), enzymatic activity, plasma protein binding and cerebral blood flow. Hydrophilic molecules such as proteins and peptides enter the brain through specific and saturable receptor-mediated transport mechanisms such as glucose transporter-1 (GLUT-1), insulin transporter and transferrin transporter. These endogenous transporters are expressed at the luminal and abluminal endothelial cell membranes. Among these transport mechanisms, receptor-mediated transcytosis has been extensively studied to deliver drugs into the brain. The dendrimer compositions and methods of the present application are suitable for delivering antidepressant and/or antipsychotic agents across the blood-brain barrier via one or more of the above-described mechanistic routes.

ii. The Blood Nerve Barrier (BNB)

The blood nerve barrier (BNB) defines the physiological space within which the axons, Schwann cells, and other associated cells of a peripheral nerve function, thereby ensuring proper function of peripheral nerves, and maintenance of homeostasis of the endoneurial environment. The BNB consists of the endoneurial microvessels within the nerve fascicle and the investing perineurium. Tight junctions between endothelial cells and between pericytes in endoneurial vasculature isolate the endoneurium from the blood, thus preventing uncontrollable leakage of molecules and ions from the circulatory system to the peripheral nerves. In addition, a diffusion barrier exists within the perineurium formed by tight junctions between the neighboring perineurial cells and basement membranes surrounding each perineurial cell layer. The endoneurial capillaries and the perineurial passage are the restrictive barriers which separate the endoneurial extracellular environment of peripheral nerves from both the epineurial perifascicular space and the systemic circulation, thus protecting the endoneurial microenvironment from drastic concentration changes in the vascular and other extracellular spaces.

For drug targets located in peripheral nerves, the BNB can be problematic because of the potential to restrict or prevent drugs from reaching their site of action, thus negatively affecting drug efficacy. In addition, transporter expression profiles in peripheral nerves can be very different from those in the central nervous system. The dendrimer compositions of the present application may be used to improve permeability of antidepressant and/or antipsychotic agents across the BNB, thereby improving delivery of antidepressant and/or antipsychotic agents to peripheral nerve targets.

iii. The Blood-CSF Barrier

The composition may be used to improve delivery of antidepressant and/or antipsychotic agents to target sites via the blood-cerebrospinal fluid barrier (blood-CSF barrier) and the ventricles. The choroid plexus is a vascular tissue found in all cerebral ventricles. The functional unit of the choroid plexus, composed of a capillary enveloped by a layer of differentiated ependymal epithelium. Unlike the capillaries that form the blood-brain barrier, choroid plexus capillaries are fenestrated and have no tight junctions. The endothelium, therefore, does not form a barrier to the movement of small molecules. Instead, the blood-CSF barrier at the choroid plexus is formed by the epithelial cells and the tight junctions that link them. The other part of the blood-CSF barrier is the arachnoid membrane, which envelops the brain. The cells of this membrane also are linked by tight junctions.

The CSF spaces and the cerebral structures adjacent to CSF compartments are pharmacological targets of interest in CNS diseases. For example, the subarachnoid, perivascular, or periventricular spaces are areas of pathogenic lymphocyte, monocyte, and neutrophil accumulation in neuroinflammatory disorders such as multiple sclerosis and related experimental autoimmune encephalitis, or virus-induced neurological disorders including neuroaids and CMV infection. Foci of B-cells detected in different CNS autoimmune diseases and producing potentially deleterious antibodies are thought to be mainly localized in leptomeninges. Therefore, in some forms, the dendrimer compositions may be used to deliver antidepressant and/or antipsychotic agents to areas of interest via the blood-CSF spaces connected with deep cervical lymph nodes for ameliorating or treating symptoms associated with neuroinflammatory disorders.

In some forms, the dendrimer compositions may be used to deliver antidepressant and/or antipsychotic agents to target sites for ameliorating or treating symptoms and conditions associated with vascular degeneration. For example, cerebral amyloid angiopathy induces degenerative vascular changes, driven by amyloid beta (AB) peptide, cystatin c, transthyretin, or gelsolin deposits around penetrating vessels. The deposits are accessible through interconnected CSF/perivascular spaces.

In some forms, the dendrimer compositions may be used to deliver antidepressant and/or antipsychotic agents to target sites for ameliorating or treating symptoms and conditions associated with tumor development. For example, periventricular tumors including meningiomas, pharmacoresistant ependymomas, and leptomeningeal metastases from peripheral primary tumors, are all in direct contact with CSF. The blood-tumor barrier is often considered leaky, as a result of the lessened efficacy of tight junctions that allows contrast enhancement in magnetic resonance imaging. However, many antidepressant and/or antipsychotic agents are lipophilic and are prevented from crossing the BBB by multidrug resistance (MDR) efflux proteins controlling the transcellular pathway. In a number of periventricular tumors such as ependymomas, MDR proteins remain well expressed at the blood-tumor barrier. Therefore, in some forms, the dendrimer compositions may be used to leverage pharmacological pressure from the CSF to achieve therapeutic concentrations of antidepressant and/or antipsychotic agents within the tumoral tissue.

b. Improve Target-Specific Binding

compositions may be used to deliver the antidepressant and/or antipsychotic agents with increased binding affinity and specificity to one or more receptors for modulation of the serotonin (5HT) receptors e.g., 5HT-1A, 5HT-2B, 5HT-2A, 5HT-2B, 5HT-2C, 5HT-3, 5HT-4. one or more norepinephrine (NE) receptors e.g., α_{1A} adrenergic receptor, α_{1B}-adrenergic receptor, α_{1D}-adrenergic receptor, α_{2A}-adrenergic receptor, α_{2B}-adrenergic receptor, α_{2C}-adrenergic receptor, β₁-adrenergic receptor, and β₂-adrenergic receptor, monoamine transporters e.g., the serotonin reuptake transporter (SERT) and/or the norepinephrine transporter (NET). The dendrimer compositions may be used to deliver the antidepressant and/or antipsychotic agents with increased binding affinity and specificity to one or more receptors for modulation of one or more of the GLUT1, GLUT2, GLUT3, GLUT4, GLUT5, GLUT6, GLUT7, GLUT8, GLUT9, GLUT10, GLUT11, GLUT12, GLUT13, and GLUT14 transporters.

i. Improve Serotonin Receptor Binding

The dendrimer compositions may improve binding to one or both serotonin receptors to modulate signaling in a cell-specific and tissue-specific manner.

In the central nervous system (CNS), serotonin is almost exclusively produced in neurons originating in the raphe nuclei located in the midline of the brainstem. These serotonin-producing neurons form the largest and most complex efferent system in the human brain. The most caudal raphe innervates the spinal cord, while the more rostral raphe, the dorsal raphe nucleus, and the medial raphe nucleus, innervate much of the rest of the CNS by diffuse projections. Almost every cell in the brain is close to a serotonergic fiber, and nearly all behaviors as well as many other brain functions are regulated by serotonin.

Both within the CNS and throughout the body, serotonin plays a number of roles in vascular biology, ranging from the control of vascular resistance and blood pressure to the control of hemostasis and platelet function. Serotonin causes vasoconstriction or vasodilation in different vascular beds depending on the particular receptors that are expressed in each vessel wall and surrounding smooth muscle tissue. Therefore, in some embodiments, the dendrimer-antidepressant compositions can be used to activate 5-HT1B receptors on cerebral blood vessels to facilitate vasodilation, resulting in the analgesic effects for migraine conditions.

Platelets have significant vesicular serotonin stores but lack the enzymes to synthesize serotonin; instead, they take up serotonin from the plasma via the serotonin transporter. Serotonin is then secreted by the platelet dense granules during platelet activation and plays a role in promoting platelet aggregation and vasoconstriction of surrounding blood vessels, facilitating hemostasis. Intracellular serotonin also facilitates platelet activation through covalent linkage to small G proteins via tissue transglutaminase. This modification constitutively activates G protein-dependent signaling pathways and stimulates platelet aggregation. In addition, serotonin is covalently cross-linked to a variety of adhesion proteins and clotting factors on the platelet cell surface, a process essential for the activation of a subset of platelets. Thus, serotonin also works by noncovalent interactions with membrane-bound receptors.

Serotonin regulates several different aspects of cardiac function, ranging from electrical conduction to valvular closure to post-MI remodeling. Therefore, in some forms, a dendrimer-SSRI composition can be administered to decrease myocardial infarction risk. For example, in some forms, the compositions can be used as 5-HT4 antagonists to help improve cardiac function and block pathological remodeling in congestive heart failure. In other forms, the compositions can be used as 5-HT2A antagonists to treat vasospastic angina and ischemic heart disease, and/or as 5-HT3 antagonists to treat post-MI pain.

ii. Improve Noradrenergic Receptor Binding

The dendrimer compositions may improve binding to one or both noradrenaline receptors to modulate signaling in a cell-specific and tissue-specific manner.

Norepinephrine, also known as noradrenaline, is a neurotransmitter of the brain that plays an essential role in the regulation of arousal, attention, cognitive function, and stress reactions. It also functions as a hormone peripherally as part of the sympathetic nervous system in the “fight or flight” response. During states of stress or anxiety, norepinephrine and epinephrine are released and bind to adrenergic receptors throughout the body which exert effects such as dilating pupils and bronchioles, increasing heart rate and constricting blood vessels, increasing renin secretion from the kidneys, and inhibiting peristalsis. The noradrenergic system plays a role in the pathogenesis of some significant neuropsychiatric disorders and has been an important pharmacologic target in various psychiatric, neurologic, and cardiopulmonary disorders.

The central noradrenergic system is composed of two primary ascending projections that originate from the brainstem: The dorsal noradrenergic bundle (DNB), and the ventral noradrenergic bundle (VNB). The DNB originates from A6 locus coeruleus, located in the dorsal pons, and is composed of primarily noradrenergic neurons. It functions as the predominant site of norepinephrine production in the central nervous system. It sends projections to innervate the cerebral cortex, hippocampus, and cerebellum exclusively and has projections that overlap with projections from the VNB to innervate areas of the amygdala, hypothalamus, and spinal cord. The VNB originates from nuclei in the pons and medulla and sends projections to innervate the amygdala, hypothalamus, and areas of the midbrain and medulla.

The sympathetic nervous system and neuroendocrine chromaffin cells (located in the adrenal medulla) are primarily responsible for the synthesis and exocytosis of norepinephrine and other catecholamines into the blood circulation. The hormones act on alpha- and beta-adrenergic receptors of smooth muscle cells and adipose tissue located throughout the body.

Following an action potential into the presynaptic terminal, voltage-gated calcium channels are stimulated and bring an influx of calcium from the extracellular to intracellular space. This influx causes norepinephrine (stored in vesicles) to bind the cell membrane and get released into the synaptic cleft via exocytosis. Norepinephrine can then go on to bind three main receptors: alpha1 (alpha-1), alpha-2, and beta receptors. These receptors classify as G-protein coupled receptors with either inhibitory or excitatory effects and different binding affinities to norepinephrine.

Alpha-1 receptors further subdivide into alpha-la, alpha-1b, and alpha-1d receptors. These receptors are located postsynaptically in regions of the brain including the locus coeruleus, olfactory bulb, cerebral cortex, dentate gyrus, amygdala, and thalamus. Alph-1 receptors have intermediate binding affinity to norepinephrine and couple to the Gq protein signaling pathway. In this pathway, phospholipase C (PLC) is activated to convert phosphatidylinositol 4,5-bisphosphate (PIP2) to inositol 1,4,5-trisphosphate (IP3) and diacylglycerol (DAG) on the cell membrane. IP3 is released to the cytosol and binds to transmembrane IP3 receptors located on the endoplasmic reticulum (ER) which functions as a calcium channel. When bound, the receptor undergoes a conformational change leading to the release of calcium from the ER to the cytosol. DAG remains in the cell membrane and positively regulates protein kinase C (PKC), which functions to phosphorylate other proteins. These combined effects produce excitatory cellular effects.

Alpha-2 receptors subdivide into alpha-2a, alpha-2b, and alpha-2c receptors. These receptors are located both presynaptically and postsynaptically in regions of the brain including locus coeruleus, amygdala, and hypothalamus. These receptors have the highest binding affinity to norepinephrine and couple to the Gi/o protein signaling pathway. In this pathway, cAMP levels are decreased thereby leading to decreased adenylyl cyclase activity, producing inhibitory cellular effects. Presynaptic noradrenergic terminals contain alpha-2 autoreceptors which prevent further release of norepinephrine.

Beta receptors subdivide into beta-1, beta-2, and beta-3 receptors. These receptors are in various regions of the brain, with beta-1 and beta-2 receptors being most prevalent in the cerebral cortex. These receptors have the lowest binding affinity to norepinephrine and couple to the Gs protein signaling pathway. In this pathway, cAMP levels increase leading to protein kinase A (PKA) activation which goes on to phosphorylate other proteins inside the cell and leads to excitatory cellular effects. Beta-2 receptors also couple to Gi protein signaling pathways. Beta-3 receptors are present in adipose tissue.

In the adrenal medulla, acetylcholine stimulates adrenaline and noradrenaline release. Acetylcholine binds to nicotinic receptors located on adrenal chromaffin cells, which generate action potentials sustained by voltage-gated sodium and potassium channels. This action potential triggers calcium influx into the cytosol, leading to norepinephrine vesicles binding to the cell membrane leading to the release of norepinephrine into the blood circulation where travel to bind alpha and beta receptors on smooth muscle and adipose cells.

Norepinephrine can be degraded intracellularly or in the synaptic cleft by the enzymes monoamine oxidase (MAO) or catechol-O-methyltransferase (COMT). MAO oxidizes norepinephrine while COMT metabolizes deaminated norepinephrine through O-methylation. MAO and COMT are found in adrenal chromaffin cells, while sympathetic nerves contain MAO only. COMT is found in all organs. The liver is responsible for the complete degradation of norepinephrine to vanillylmandelic acid (VMA).

### B. Conditions to be Treated

The compositions are suitable for treating one or more diseases, conditions, and injuries in the central and peripheral nervous system. The compositions can also be used for treatment of a variety of diseases, disorders and injury including mental health disorders, gastrointestinal disorders, cardiovascular disorders, and/or treatment of other tissues where the nerves play a role in the disease or disorder. The compositions and methods are also suitable for prophylactic use. For example, the compositions may be administered to a patient in need thereof to ameliorate, treat or prevent symptoms associated with a variety of disorders, diseases, and conditions including, but not limited to, anxiety disorders, mood disorders, eating disorders, ADHD, personality disorders, stress disorders, and/or psychotic disorders. In some forms, when the dendrimer is complexed to one or more antidepressants, the compositions can be administered to treat one or more neurological disorders such as mental health disorders e.g., mood disorders, anxiety disorders, eating disorders, substance-related disorders, and post-traumatic disorders; degenerative disorders e.g., Parkinson's disease and Alzheimer's disease; pain disorders e.g., neuropathic pain, gastrointestinal disorders, and/or cardiovascular disorders. In some forms, when the dendrimer is complexed to one or more antipsychotics, the compositions can be administered to treat psychoses that occurs in bipolar disorder, schizophrenia, and/or degenerative disorders. In other forms, the compositions may be administered to a subject in need for stabilizing moods e.g., in bipolar disorder, reducing anxiety in anxiety disorders and reducing tics in Tourette syndrome. In yet other forms, the dendrimer is complexed to one or more antidepressants and/or antipsychotics to treat non-neural diseases such as gastrointestinal and cardiovascular diseases.

The dendrimer complexes are administered in a dosage unit amount effective to treat or alleviate conditions associated with the pathological conditions of the central and peripheral nervous system. For example, the dendrimer complexes are administered in a dosage unit amount effective to treat or alleviate conditions associated with pathological conditions that affect neurons, microglia, and/or astrocytes. Generally, by targeting these cells, the dendrimers deliver agent specifically to treat neuroinflammation.

In preferred embodiments, the compositions may include glucose or hydroxyl dendrimers 5 nm or smaller in diameter and conjugated to antidepressant, antipsychotic, or other agents that act via modulation of monoaminergic neurotransmission for the treatment of mental health and CNS disorders. In other preferred embodiments, the compositions may include glucose or hydroxyl dendrimers larger than 5 nm in diameter and conjugated to antidepressant, antipsychotic, or other agents that act via modulation of monoaminergic neurotransmission for the treatment of peripheral nervous system disorders.

### 1. Mental Health Disorders and Conditions

The compositions and methods are suitable for the treatment of a variety of mental health disorders and conditions including but not limited to affective or mood disorders, anxiety disorders, childhood disorders, eating disorders, personality disorders, schizophrenia and other psychotic disorders, and substance-related disorders.

a. Affective or Mood Disorders

Affective or mood disorders are described by marked disruptions in emotions (severe lows called depression or highs called hypomania or mania). These include bipolar disorder, cyclothymia, hypomania, major depressive disorder, disruptive mood dysregulation disorder, treatment-resistant depression, persistent depressive disorder, premenstrual dysphoric disorder, seasonal affective disorder, depression related to medical illness, depression induced by substance use or medication.

In some forms, the compositions may be administered to a subject in need to treat, prevent, or manage the symptoms of Major Depressive Disorder including but not limited to persistently low or depressed mood, anhedonia or decreased interest in pleasurable activities, feelings of guilt or worthlessness, lack of energy, poor concentration, appetite changes, psychomotor retardation or agitation, sleep disturbances, and/or suicidal thoughts.

b. Anxiety Disorders

Anxiety disorders differ from normal feelings of nervousness or anxiousness and involve excessive fear or anxiety. Anxiety disorders include generalized anxiety disorder, panic disorder, social anxiety disorder, and various phobia-related disorders.

Generalized anxiety disorder (GAD) usually involves a persistent feeling of anxiety or dread, which can interfere with daily life. It is not the same as occasionally worrying about things or experiencing anxiety due to stressful life events. People living with GAD experience frequent anxiety for months, if not years. The compositions and methods are suitable for the treatment of one or more symptoms of GAD, including but not limited to restlessness, fatigue, difficulty concentrating, irritability, headaches, muscle aches, stomach aches, or unexplained pains, excessive worry, sleep issues e.g., difficulty falling or staying asleep.

In some forms, the compositions may be administered to a patient in need thereof to treat, prevent, or manage one or more symptoms associated with a panic disorder. Panic Disorder is an anxiety disorder characterized by unexpected and repeated episodes of intense fear accompanied by physical symptoms that may include chest pain, heart palpitations, shortness of breath, dizziness, or abdominal distress, or sense of losing control even when there is no clear danger or trigger. Individuals with panic disorders often worry about when the next attack will happen and actively try to prevent future attacks by avoiding places, situations, or behaviors they associate with panic attacks. Panic attacks can occur as frequently as several times a day or as rarely as a few times a year. The compositions and methods are suitable for the treatment of one or more symptoms of panic attacks including but not limited to heart palpitations, excess sweating, trembling, or tingling, chest pain, and difficulty controlling feelings e.g., feelings of impending doom and feelings of being out of control.

Social anxiety disorder is an intense, persistent fear of being watched and judged by others. For people with social anxiety disorder, the fear of social situations may feel so intense that it seems beyond their control. For some people, this fear may get in the way of going to work, attending school, or doing everyday things. The compositions and methods are suitable for the treatment of one or more symptoms of social anxiety disorders including but not limited to excess blushing, sweating, or trembling, heart palpitations, stomachaches, rigid body posture or speaking with an overly soft voice, and feelings of self-consciousness or fear of negative judgement.

A phobia is an intense fear of—or aversion to—specific objects or situations. Although it can be realistic to be anxious in some circumstances, the fear people with phobias feel are out of proportion to the actual danger caused by the situation or object. The compositions and methods are suitable for the treatment of one or more symptoms of phobias including but not limited to irrational or excessive worry about encountering the feared object or situation, immediate intense anxiety upon encountering the feared object or situation, and enduring unavoidable objects and situations with intense anxiety.

c. Eating Disorders

Eating disorders are serious and often fatal illnesses that are associated with severe disturbances in people's eating behaviors and related thoughts and emotions. Eating disorders include preoccupation with food, body weight, and shape. Common eating disorders include anorexia nervosa, bulimia nervosa, and binge-eating disorder.

Anorexia nervosa is a condition where people avoid food, severely restrict food, or eat very small quantities of only certain foods. They also may weigh themselves repeatedly. Even when dangerously underweight, they may see themselves as overweight. There are two subtypes of anorexia nervosa: a restrictive subtype and a binge-purge subtype. People with the restrictive subtype of anorexia nervosa severely limit the amount and type of food they consume. People with the binge-purge subtype of anorexia nervosa also greatly restrict the amount and type of food they consume. In addition, they may have binge-eating and purging episodes-eating large amounts of food in a short time followed by vomiting or using laxatives or diuretics to get rid of what was consumed. Symptoms of anorexia nervosa including but not limited to thinning of the bones (osteopenia or osteoporosis), mild anemia and muscle wasting and weakness, brittle hair and nails, dry and yellowish skin, growth of fine hair all over the body (lanugo), severe constipation, low blood pressure, slowed breathing and pulse, damage to the structure and function of the heart, brain damage, multiorgan failure, drop in internal body temperature, causing a person to feel cold all the time, lethargy, sluggishness, or feeling tired all the time, and infertility.

Bulimia nervosa is a condition where people have recurrent and frequent episodes of eating unusually large amounts of food and feeling a lack of control over these episodes. This binge-eating is followed by behavior that compensates for the overeating such as forced vomiting, excessive use of laxatives or diuretics, fasting, excessive exercise, or a combination of these behaviors. People with bulimia nervosa may be slightly underweight, normal weight, or over overweight. Symptoms of bulimia nervosa include chronically inflamed and sore throat, swollen salivary glands in the neck and jaw area, worn tooth enamel and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (too low or too high levels of sodium, calcium, potassium, and other minerals) which can lead to stroke or heart attack.

d. Schizophrenia

The compositions and methods are suitable for the treatment of symptoms associated with schizophrenia.

Schizophrenia is characterized by significant impairments in the way reality is perceived and changes in behavior related to persistent delusions i.e., the person has fixed beliefs that something is true, despite evidence to the contrary; persistent hallucinations i.e., the person may hear, smell, see, touch, or feel things that are not there; experiences of influence, control or passivity i.e., the experience that one's feelings, impulses, actions, or thoughts are not generated by oneself, are being placed in one's mind or withdrawn from one's mind by others, or that one's thoughts are being broadcast to others; disorganized thinking, which is often observed as jumbled or irrelevant speech; highly disorganized behavior e.g. the person does things that appear bizarre or purposeless, or the person has unpredictable or inappropriate emotional responses that interfere with their ability to organize their behavior; “negative symptoms” such as very limited speech, restricted experience and expression of emotions, inability to experience interest or pleasure, and social withdrawal; and/or extreme agitation or slowing of movements, maintenance of unusual postures.

Antipsychotics are the mainstay in the pharmacologic treatment of schizophrenia. Therefore, the compositions are suitable for treating one or more of the above positive, negative, cognitive, disorganization, and mood symptoms in a subject in need thereof.

### 2. Neurological and Neurodegenerative Diseases

The compositions and methods are suitable for the treatment of symptoms associated with neurological and neurodegenerative diseases.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target (Hagberg, H et al., Annals of Neurology 2012, 71, 444; Vargas, D L et al., Annals of Neurology 2005, 57, 67; and Pardo, C A et al., International Review of Psychiatry 2005, 17, 485). Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to deliver active agents for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder or peripheral symptoms resulting from a neurological or neurodegenerative disease or disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Multiple Sclerosis (MS), post-encephalitic dementia, cancer and chemotherapy-associated cognitive impairment and dementia, and depression-induced dementia and pseudodementia.

### 3. Pain

The compositions and methods are suitable for the treatment of neuropathic and/or non-neuropathic pain associated with various disorders, such as for example, complex regional pain syndrome, peripheral neuropathy, multiple sclerosis, and sciatica. For example, tricyclic antidepressants (TCAs) are effective in treating neuropathic (chronic) nerve pain. Chronic nerve pain, also known as neuropathic pain, is caused by nerve damage or other problems with the nerves, and is often unresponsive to regular painkillers, such as paracetamol. In some forms, the compositions can be used to treat cases of chronic pain that do not involve nerves (non-neuropathic pain). For example, the dendrimer compositions can be complexed with a TCA, SSRI and/or serotonin-noradrenaline reuptake inhibitors (SNRIs) and used to treat chronic non-neuropathic pain. Conditions that cause non-neuropathic pain which may benefit from treatment with compositions include but are not limited to fibromyalgia, chronic back pain, and chronic neck pain.

### 4. Gastrointestinal Disorders

The compositions and methods are suitable for the treatment of gastrointestinal disorders and/or digestive diseases.

Functional gastrointestinal (GI) and motility disorders are the most common GI disorders in the general population. Functional GI disorders are disorders of gut-brain interaction. It is a group of disorders classified by GI symptoms related to any combination of the following: motility disturbance, visceral hypersensitivity, altered mucosal and immune function, altered gut microbiota, and altered central nervous system (CNS) processing such as difficulties in the brain's ability to regulate painful signals from the GI tract. The term “functional” is generally applied to disorders where the body's normal activities in terms of the movement of the intestines, the sensitivity of the nerves of the intestines, or the way in which the brain controls some of these functions is impaired. In some forms, the compositions can be administered to treat a bowel disorder and/or abdominal pain e.g., irritable bowel syndrome (IBS), functional abdominal bloating/distension, constipation, diarrhea, and/or opioid induced constipation. With IBS, a person's awareness and interpretation of these activities may be abnormal (abnormal perception). In some form, the compositions can be administered to treat a centrally mediated disorder of gastrointestinal pain such as centrally mediated abdominal pain syndrome (CAPS) and/or narcotic bowel syndrome (NBS)/opioid-induced GI hyperalgesia.

### 5. Irritable Bowel Syndrome

IBS is characterized by abnormal changes in the movement of the muscles of the intestines (abnormal motility), an increase in the sensations produced by intestinal activity (visceral hypersensitivity), and brain-gut dysfunction, especially difficulties in the brain's ability to regulate painful signals from the GI tract. Instead of the normal muscular activity (motility) of digestion, IBS patients may experience spasms and cramping. If the motility is too fast it may result in diarrhea, and if it is too slow it might result in constipation. These two conditions may also produce abdominal discomfort or pain in IBS patients. Abnormal motility can also be associated with abdominal cramping, belching, urgency, or other unpleasant GI symptoms.

For IBS patients, there can also be increased sensitivity of the nerves in the GI tract. This can develop after a gastrointestinal infection or an operation that causes injury to the nerves in the intestine. This results in a lower threshold for experiencing intestinal sensations, leading to abdominal discomfort or pain. In those with visceral hypersensitivity, the stretch put on the intestines from eating even small amounts of food may produce discomfort.

When the nerve impulses from the gut reach the brain, they may be experienced as more severe or less severe based on the regulatory activities of the brain-gut axis. Signals of pain or discomfort travel from the intestines up to the brain. The brain can “turn down” the pain by sending signals that block the nerve impulses produced in the GI tract. This ability to turn down the pain is impaired in patients with IBS. In addition, the pain can become more severe when an individual is experiencing psychological distress. Often this may occur because of stresses in life or even the stress and frustration of the GI symptoms. This brain-gut dysfunction can be remedied with either psychological treatments or antidepressants or a combination of both.

Therefore, the dendrimer-antidepressant compositions can be used as effective analgesics (drugs that reduce pain) to treat symptoms of IBS and other functional GI disorders. In some forms, the dendrimer-antidepressant compositions are effective to treat abdominal pain and reduce other IBS symptoms, such as diarrhea, constipation, bloating, nausea, or urgency. For example, the dendrimer-antidepressant conjugates can help regulate abnormal bowel functions like diarrhea and, constipation, as well as other

IBS symptoms. In other forms, a tricyclic antidepressant (TCA) can be included in the dendrimer-antidepressant conjugate to help with diarrhea e.g., amitriptyline (Elavil), Imipramine (Tofranil), Desipramine (Norpramin), and Nortriptyline, and (Pamelor). In yet another examples, the dendrimer-antidepressant conjugate can include a serotonin reuptake inhibitor (SSRI) to help treat constipation. In an exemplary embodiment, the dendrimer-antidepressant conjugate can help with other problems such as anxiety and depression, which are often associated with chronic painful disorders.

### 6. Sleep Disorders

The compositions and methods are suitable for the treatment or management of symptoms associated with sleep disorders and/or sleep disruption including but not limited to insomnia, restless legs syndrome, narcolepsy and sleep apnea.

In some forms, the compositions and methods are also suitable for the treatment of symptoms associated with narcolepsy. Narcolepsy is a disorder of rapid onset rapid eye movement (REM) sleep characterized by excessive daytime sleepiness (EDS), frequent uncontrollable sleep attacks as well as sleep fragmentation and can be associated with cataplexy, sleep paralysis, and hypnagogic hallucinations. There are two types: narcolepsy type 1 (formerly narcolepsy with cataplexy) and narcolepsy type 2 (formerly narcolepsy without cataplexy). In some forms, the compositions and methods may be administered to a patient in need thereof to treat or manage one or more symptoms of cataplexy including seizures (especially atonic seizures), periodic paralysis, cardiogenic syncope, orthostatic syncope, neurogenic syncope and or psychotic symptoms. In some forms, the compositions and methods may be administered to a patient in need thereof to treat or manage excessive daytime sleepiness in a narcoleptic patient. In some forms, the compositions and methods may be administered to a patient in need thereof to treat or manage depression and/or anxiety symptoms in a narcoleptic patient.

### 7. Neurodevelopment and Attention Deficit Disorders

The compositions and methods are suitable for the treatment or management of symptoms associated with an attention deficit disorder e.g., attention deficit hyperactivity disorder, autism spectrum disorder, Tourette's syndrome, sensory integration disorders, auditory processing disorders, and other specific learning difficulties.

In some forms, the compositions may be administered to a patient in need thereof to treat or manage the symptoms associated with a developmental disorder such as autism spectrum disorders. Autism spectrum disorder (ASD) encompasses a spectrum of neurodevelopmental disabilities. This spectrum is characterized by repetitive patterns of behavior, interests, activities, and problems in social interactions. ASD is a complicated neurodevelopmental disorder that is characterized by behavioral and psychological problems in children. These children become distressed when their surrounding environment is changed because their adaptive capabilities are minimal. The symptoms are present from early childhood and affect daily functioning. Children with ASD have co-occurring language problems, intellectual disabilities, and epilepsy at higher rates than the general population.

In some forms, the compositions may be administered to a patient in need thereof to treat or manage the symptoms associated with Attention-Deficit/Hyperactivity Disorder (ADHD). ADHD is one of the most common neurodevelopmental disorders and is typically first diagnosed in childhood and often lasts into adulthood. Individuals with ADHD may have trouble paying attention, controlling impulsive behaviors (may act without thinking about what the result will be), or be overly active. Symptoms of ADHD include but are not limited to inattention e.g., disorganization, problems staying on task, constant daydreaming, and not paying attention when spoken to directly; impulsivity such as spur-of-the-moment decisions without thinking about the chance of harm or long-term effects, often acting quickly to get an immediate reward, and regularly interrupting others; and hyperactivity such as squirming, fidgeting, tapping, talking, and constant movement, especially in situations where it's not appropriate.

Tourette syndrome referred to as Tourette disorder is a common neurodevelopmental disorder affecting up to 1% of the population. It is characterized by multiple motor and vocal tics and starts in childhood. In some forms, the compositions may be administered to a patient in need thereof to treat or manage the symptoms associated Tourette syndrome including but not limited to simple tics such as sudden, brief, repetitive movements that involve a limited number of muscle groups e.g. eye blinking and other eye movements, facial grimacing, shoulder shrugging, and head or shoulder jerking, repetitive throat clearing, sniffing, barking, or grunting sounds, and/or complex tics such as distinct, coordinated patterns of movement involving several muscle groups e.g., facial grimacing combined with a head twist and a shoulder shrug, sniffing or touching objects, hopping, jumping, bending, or twisting, repeating one's own words or phrases, repeating others' words or phrases (called echolalia), or more rarely, using vulgar, obscene, or swear words (called coprolalia).

### C. Dosage and Effective Amounts

In some in vivo approaches, the dendrimer complexes are administered to a subject in a therapeutically effective amount. The term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease or disorder, and the treatment being affected.

Generally, the dose of the compositions can be about 0.001 to about 100 mg/kg body weight of the subject being treated, from about 0.01 to about 100 mg/kg body weight, from about 0.1 mg/kg to about 10 mg/kg, and from about 0.5 mg to about 5 mg/kg body weight. The subjects are typically mammals, most preferably, humans.

For example, dendrimer complex compositions can be in an amount effective to deliver one or more active agents to cells at or nearby the site of inflammation, particularly inflammation of the central nervous system, or inflammation of the eye. Therefore, in some embodiments, the dendrimer complex compositions including one or more active agent are in an amount effective to ameliorate inflammation in a subject. In a preferred embodiment, the effective amount of dendrimer complex compositions does not induce significant cytotoxicity in the cells of a subject compared to an untreated control subject. Preferably, the amount of dendrimer complex compositions is effective to prevent or reduce inflammation and/or further associated symptoms of a disease or disorder in a subject compared to an untreated control.

In general, the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, or weekly dosing.

In some embodiments, dosages are administered once, twice, or three times daily, or every other day, two days, three days, four days, five days, or six days to a human. In some embodiments, dosages are administered about once or twice every week, every two weeks, or every three weeks. In some embodiments, dosages are administered about once or twice every month, every two months, every three months, every four months, every five months, or every six months.

It will be understood by those of ordinary skill that a dosing regimen can be any length of time sufficient to treat the disorder in the subject. The term “chronic” means that the length of time of the dosage regimen can be hours, days, weeks, months, or possibly years.

In some embodiments, the regimen includes one or more cycles of a round of therapy followed by a drug holiday (e.g., no drug). The round of the therapy can be, for example, and of the administrations discussed above. Likewise, the drug holiday can be 1, 2, 3, 4, 5, 6, or 7 days: or 1, 2, 3, 4 weeks, or 1, 2, 3, 4, 5, or 6 months.

The dendrimer complexes can be administered in combination with one or more additional therapeutically active agents, which are known to be capable of treating conditions or diseases discussed above.

### D. Controls

The effect of dendrimer complex compositions can be compared to a control. Suitable controls are known in the art and include, for example, untreated cells or an untreated subject. In some embodiments, the control is untreated tissue from the subject that is treated, or from an untreated subject. Preferably the cells or tissue of the control are derived from the same tissue as the treated cells or tissue. In some embodiments, an untreated control subject suffers from, or is at risk from the same disease or condition as the treated subject.

### E. Combination Therapies

The dendrimer complex compositions can be administered alone, or in combination with one or more additional active agent(s), as part of a therapeutic or prophylactic treatment regime, including other antidepressant or psychedelic agents, cannabinoids, or psychedelics. The dendrimer complex compositions can be administered on the same day, or a different day than the second active agent. For example, compositions including dendrimer complex compositions can be administered on the first, second, third, or fourth day, or combinations thereof.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second).

### VI. Kits

The compositions can be packaged in kit. The kit can include a single dose or a plurality of doses of a composition including one or more antidepressant and antipsychotic agents associated with or conjugated to a dendrimer (e.g., one or more hydroxyl-terminated PAMAM dendrimers or glucose dendrimers as described in the Examples), and instructions for administering the compositions. Specifically, the instructions direct that an effective amount of the dendrimer composition be administered to an individual with a particular disease/disorder as indicated. The composition can be formulated as described above with reference to a particular treatment method and can be packaged in any convenient manner.

The present invention will be further understood by reference to the following non-limiting examples.

### EXAMPLES

### Example 1: Synthesis of Hydroxyl-Polyamidoamine (PAMAM-OH) Dendrimer Conjugated with Antipsychotics/Antidepressants

Conjugation of PAMAM-OH to one or more antipsychotics or antidepressants is described below. Selective serotonin reuptake inhibitors (SSRIs) drugs are used as exemplary agents. Exemplary SSRIs include Fluoxetine (PROZAC®), paroxetine (PAXIL®), Venlafaxine, citalopram (CELEXA®), etc.

The synthesis of PAMAM-OH-SSRI conjugates is achieved using a combination of linking chemistries and linkers (both cleavable and non-cleavable). Briefly, the surface hydroxyl groups on PAMAM-OH are modified with a linker to bring a complementary group on the surface that can further react with the complimentary group on the drug linker. On the other hand, the drug is modified by the linker to bring a complimentary functional group for reacting with dendrimer-linker. The linker on the drug is attached by cleavable or non-cleavable linkages. The examples of cleavable linkages include, esterase sensitive ester bond, glutathione sensitive disulfide bond, phosphatase-sensitive phosphodiester bond, triglycyl peptide linker (CX) capable of lysosomal release, acid cleavable hydrazine linkage etc. The examples of non-cleavable linkages include ether, amino alkyl, or amide bonds. The linkers can be amino-acids, peptides, polyethylene glycol (n=2-15), or hydrocarbon chain.

### 1. Synthesis of Hydroxyl-Terminated PAMAM Dendrimer-Fluoxetine (PROZAC®) Conjugate Using a Non-Cleavable Linkage

The PAMAM dendrimer-fluoxetine (Prozac) conjugate was prepared with a non-cleavable linkage according to the reactions illustrated in FIGS. 1A and 1B. FIGS. 1A and 1B are schematics showing an exemplary synthetic route for conjugating fluoxetine to the dendrimer using click chemistry.

### 2. Synthesis of Hydroxyl-Terminated PAMAM Dendrimer-Paroxetine (PAXIL®) Conjugate Using a Non-Cleavable Linkage

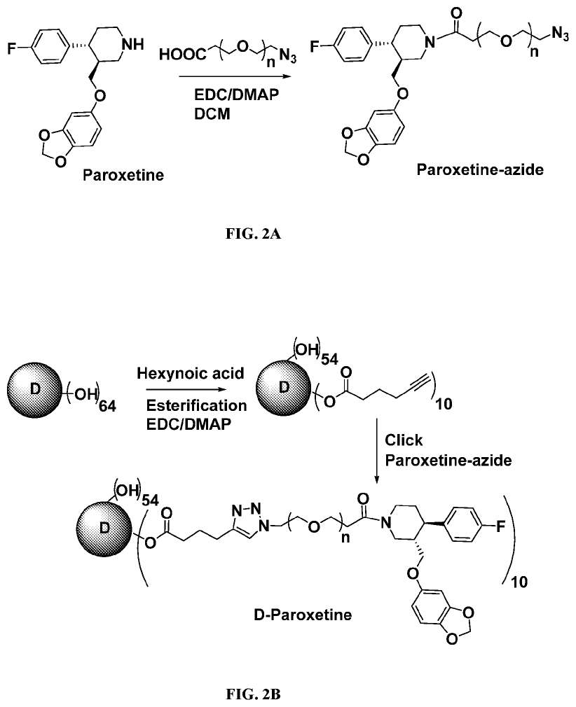

The PAMAM dendrimer-paroxetine (Paxil) conjugate was prepared with a non-cleavable linkage according to the reactions illustrated in FIGS. 2A and 2B. FIGS. 2A and 2B are schematics showing an exemplary synthetic route for conjugating paroxetine to the dendrimer using click chemistry.

### 3. Synthesis of Hydroxyl-Terminated PAMAM Dendrimer-Venlafaxine Conjugate Using an Enzyme-Cleavable Linkage

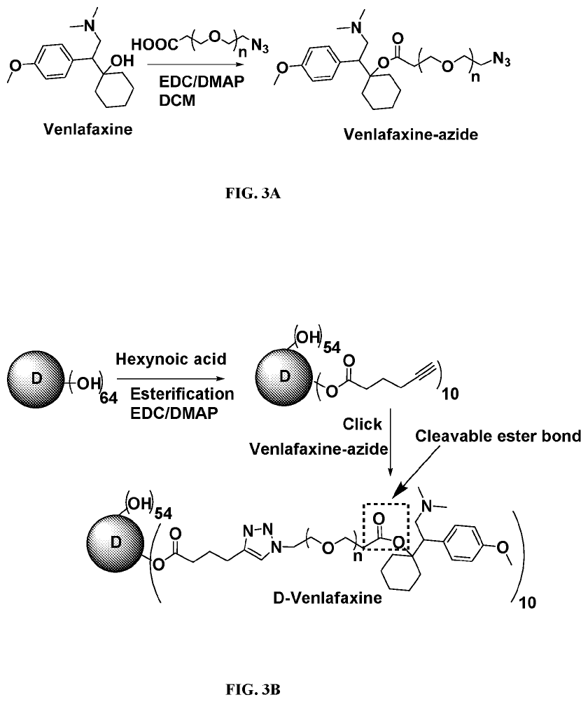

The PAMAM dendrimer-venlafaxine conjugate was prepared with an enzyme-cleavable ester linkage according to the reactions illustrated in FIGS. 3A and 3B. FIGS. 3A and 3B are schematics showing an exemplary synthetic route for conjugating venlafaxine to the dendrimer using click chemistry.

### 4. Synthesis of Hydroxyl-Terminated PAMAM Dendrimer-Venlafaxine Analog Conjugate Using a Non-Cleavable Linkage

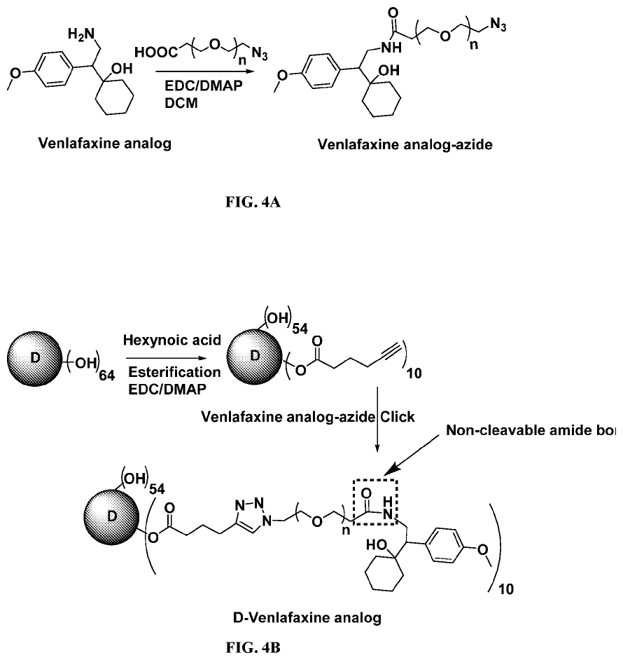

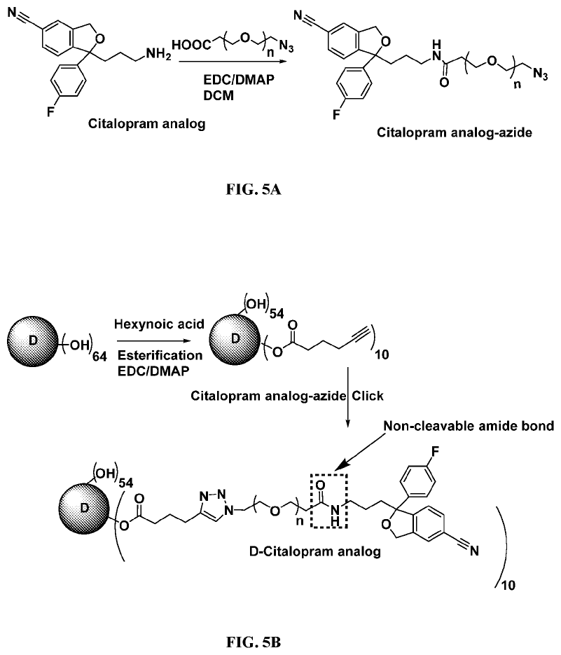

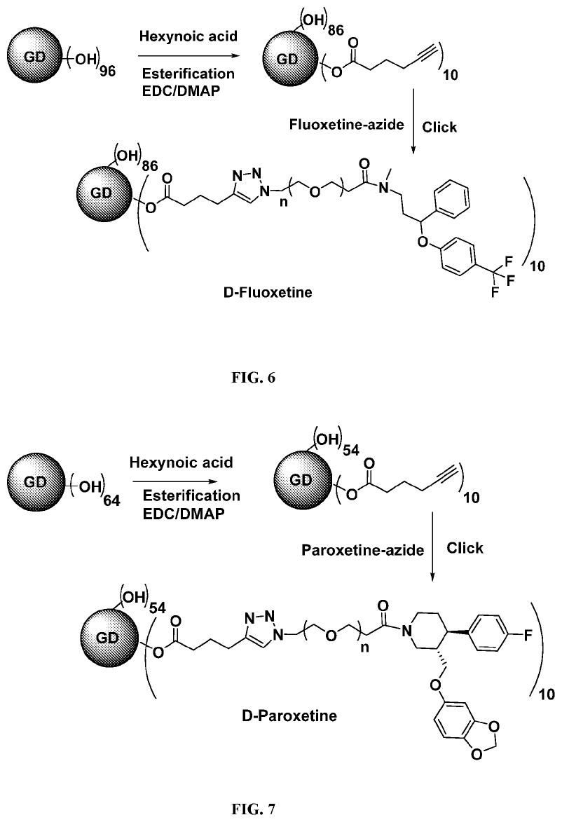

The PAMAM dendrimer-venlafaxine analog conjugate was prepared with a non-cleavable amide linkage according to the reactions illustrated in FIGS. 4A and 4B. FIGS. 4A and 4B are schematics showing an exemplary synthetic route for conjugating venlafaxine analog to the dendrimer using click chemistry.

### 5. Synthesis of Hydroxyl-Terminated PAMAM Dendrimer-Citalopram (CELEXA®) Analog Conjugate Using a Non-Cleavable Linkage

The PAMAM dendrimer citalopram (Celexa) conjugate was prepared with a non-cleavable amide linkage according to the reactions illustrated in FIGS. 5A and 5B. FIGS. 5A and 5B are schematics showing an exemplary synthetic route for conjugating citalopram to the dendrimer using click chemistry.

### Example 2: Synthesis of Glucose Dendrimer (GD) Conjugated with Antipsychotics/Antidepressants

Conjugation of glucose dendrimer (GD) to one or more antipsychotics or antidepressants is described below. Selective serotonin reuptake inhibitors (SSRIs) drugs are used as exemplary agents. Exemplary SSRIs include Fluoxetine (PROZAC®), paroxetine (PAXIL®), Venlafaxine, citalopram (CELEXA®), etc.

The synthesis of GD-SSRI conjugates is achieved using a combination of variety of linking chemistries and linkers (both cleavable and non-cleavable). Briefly, the surface hydroxyl groups on GD are modified with a linker to bring a complementary group on the surface that can further react with the complimentary group on the drug linker. On the other hand, the drug is modified by the linker to bring a complimentary functional group for reacting with dendrimer-linker. The linker on the drug is attached by cleavable or non-cleavable linkages. The examples of cleavable linkages include, esterase sensitive ester bond, glutathione sensitive disulfide bond, phosphatase-sensitive phosphodiester bond, triglycyl peptide linker (CX) capable of lysosomal release, acid cleavable hydrazine linkage etc. The examples of non-cleavable linkages include ether or amide bonds. The linkers can be amino-acids, peptides, polyethylene glycol (n=2-15), or hydrocarbon chain.

### 1. Synthesis of Glucose Dendrimer-Fluoxetine (PROZAC®) Conjugate Using a Non-Cleavable Linkage

The glucose dendrimer-fluoxetine conjugate was prepared with a non-cleavable linkage according to the reactions illustrated in FIG. 6. The synthesis of fluoxetine-azide is shown in FIG. 1A. The synthesis of glucose dendrimer and fluoxetine conjugate is achieved by the partial modification of OH groups of glucose dendrimers with a complimentary group on the surface of the glucose dendrimer which is reacted with a complimentary linker containing azide connected to the fluoxetine to generate the glucose dendrimer-fluoxetine conjugate (FIG. 6).

### 2. Synthesis of Glucose Dendrimer-Paroxetine (PAXIL®) Conjugate Using a Non-Cleavable Linkage

The glucose dendrimer-paroxetine conjugate was prepared with a non-cleavable linkage according to the reactions illustrated in FIG. 7. The synthesis of paroxetine-azide is shown in FIG. 2A. The synthesis of glucose dendrimer and paroxetine conjugate is achieved by the partial modification of OH groups of glucose dendrimers with a complimentary group on the surface of the glucose dendrimer which is reacted with a complimentary linker containing azide connected to the paroxetine to generate the glucose dendrimer-paroxetine conjugate (FIG. 7).

### 3. Synthesis of Glucose Dendrimer-Venlafaxine Conjugate Using An Enzyme-Cleavable Linkage

The glucose dendrimer-venlafaxine conjugate was prepared with an enzyme-cleavable ester linkage according to the reactions illustrated in FIG. 8. Venlafaxine was first modified with an azide as shown in FIG. 3A. The exemplary synthesis route of glucose dendrimer and venlafaxine conjugate is shown in FIG. 8.

### 4. Synthesis of Glucose Dendrimer-Venlafaxine Analog Conjugate Using a Non-Cleavable Linkage

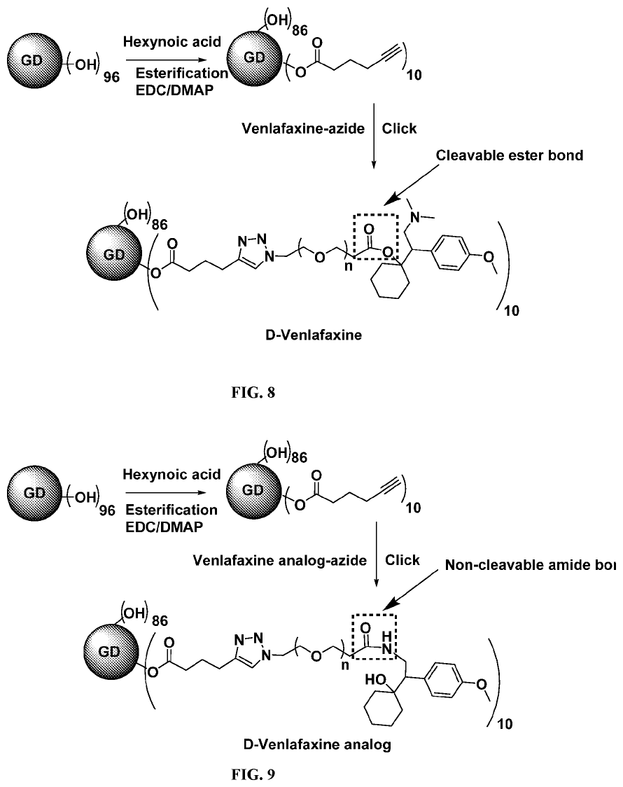

The glucose dendrimer-venlafaxine conjugate was prepared with a non-cleavable amide linkage according to the reactions illustrated in FIG. 9. Venlafaxine was first modified with an azide as shown in FIG. 4A. The exemplary synthesis route of glucose dendrimer and venlafaxine conjugate is shown in FIG. 9.

### 5. Synthesis of Glucose Dendrimer-Citalopram Analog Conjugate Using a Non-Cleavable Linkage

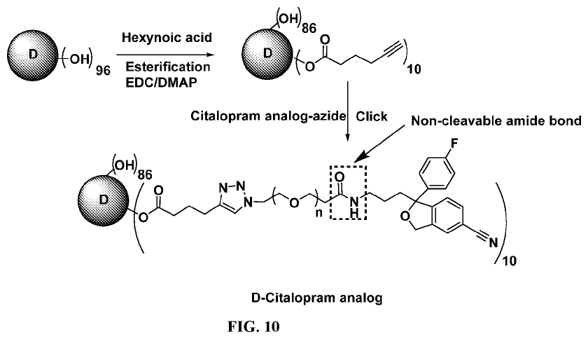

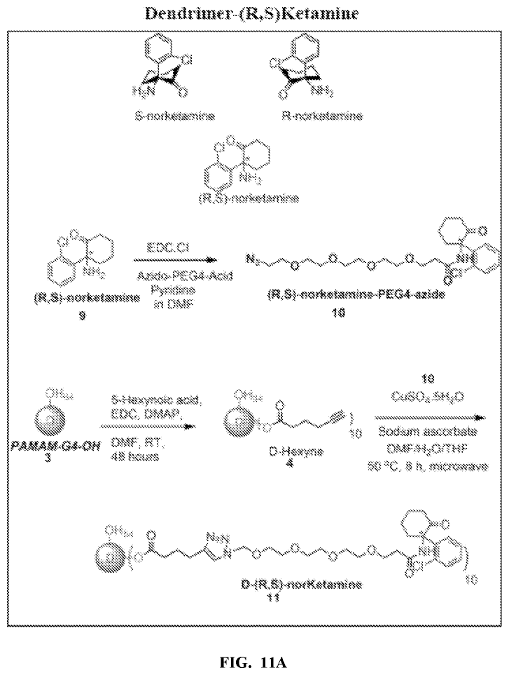

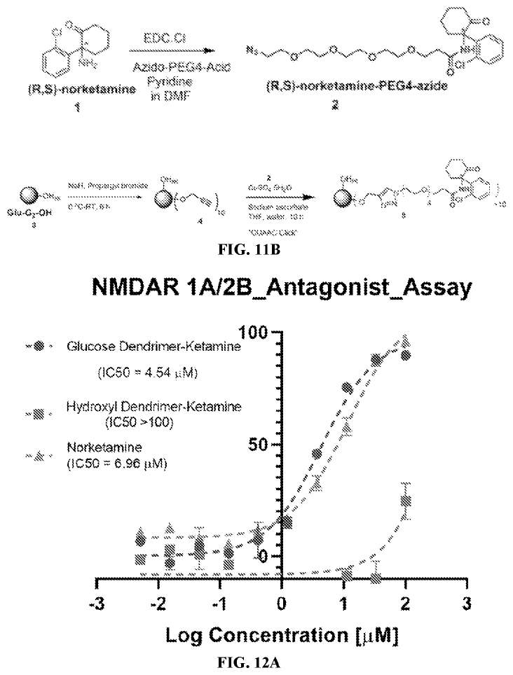

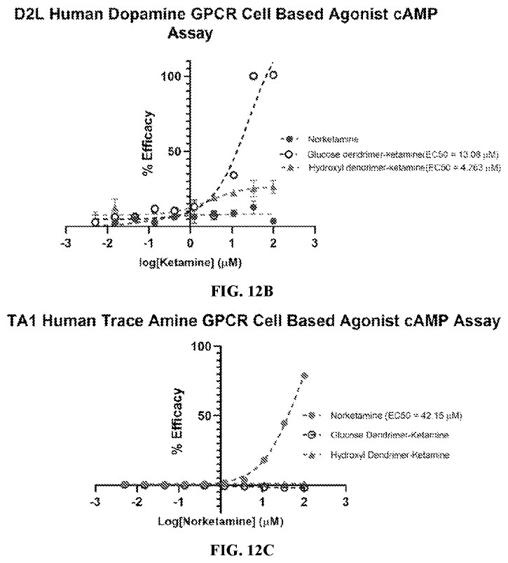

The glucose dendrimer-citalopram analog conjugate was prepared with a non-cleavable linkage according to the reactions illustrated in FIG. 10. Citalopram analog was first modified with an azide as shown in FIG. 5A. The exemplary synthesis route of glucose dendrimer and citalopram analog conjugate is shown in FIG. 10.

Unless stated otherwise, reactions were performed in flame dried glassware under a positive pressure of nitrogen using dry solvents. Commercial grade reagents and anhydrous solvents were purchased from chemical suppliers and used without further purification. 1-Ethyl-3-(3-dimethylaminopropyl) carbodiimide (EDC·HCl), N, N-diisopropylethylamine (DIPEA), 4-(dimethylamino)pyridine (DMAP) trifluoracetic acid (TFA), anhydrous dichloromethane (DCM), N,N′-dimethylformamide (DMF) were purchased from Sigma-Aldrich (St. Louis, MO, USA). Cyanine 5 (Cy5)-mono-NHS ester was purchased from Amersham Bioscience-GE Healthcare. Deuterated solvents dimethylsulfoxide (DMSO-d6), water (D₂O), and Chloroform (CDCl₃) were purchased from Cambridge Isotope Laboratories Inc. (Andover, MA). Ethylenediamine-core polyamidoamine (PAMAM) dendrimer, generation 4.0, hydroxy surface (G4-OH; diagnostic grade; consisting of 64 hydroxyl end-groups), methanol solution (13.75% w/w) was purchased from Dendritech Inc. (Midland, MI, USA). Dialysis membranes were purchased from Spectrum Laboratories Inc. (Rancho Dominguez, CA, USA). Hu308, Tryptamine, 1-(2-amino-1-(4-methoxyphenyl)ethyl) cyclohexanol, Nor-ketamine, 5-hydroxy tryptamine, psilocybin analog, psilocyn analog and cannabidiol drugs were purchased from Cayman Chemicals.

### Synthesis of Hydroxyl-PAMAM Dendrimer Drug Conjugates.

The PAMAM-G4-OH (D4-OH) dendrimer composed of about 64 terminal hydroxyl groups was used for the synthesis. After each synthetic step, the product was purified via dialysis in DMF for 24 h to eliminate small molecule impurities followed by water dialysis to remove DMF. ¹H NMR (in DMSO-d6 and D₂O) and analytical HPLC were used to confirm the intermediates and final product formation and purity. The mono-functional D4-OH was functionalized with alkyne group by treatment of 5-hexynoic acid under standard esterification conditions using EDC·HCl and 4-DMAP in DMF for 36 h at room temperature to yield the D-hexyne bifunctional dendrimer. The number of alkyne groups on dendrimer surface was chosen to be kept at ˜10-15 to maintain the overall water solubility of the conjugate. The crude product was dialyzed by 1 kDa membrane against ultrapure water for 24 h to remove low molecular weight impurities via selective diffusion across the semi-permeable dialysis membrane. The ¹H NMR and analytical HPLC were used to confirm the product formation and purity of the intermediates and final products.

### Synthesis of D-Hexyne

A solution of PAMAM G4-OH 1 (10.00 g, 0.7 mmol) in DMF (50 mL) was treated with 5-Hexynoic acid (1.40 g, 12.6 mmol), DMAP (2.41 g, 12.6 mmol) and stirred at room temperature for 5 min. Then EDC·HCl (1.54 g, 12.6 mmol) was added in portions to the reaction mixture over the period of 5 min. The reaction mixture was stirred at room temperature for 36 h. The crude product was transferred to 1 kD MW cut-off cellulose dialysis tubing and dialyzed against DMF 12 h followed by water for 24 h. The aqueous layer was frozen and lyophilized to yield D-hexyne as a hygroscopic white solid (75% yield). ¹H NMR (500 MHz, DMSO-d₆) 8.21-7.57 (m, internal amide H), 4.71 (s, GABA amide H, 50H), 4.01 (t, 22-24 CH₂), 3.5-2.1 (m, dendrimer CH₂) 1.71-1.59 (m, 22-24 CH₂). HPLC C18 retention time 4 min: purity ˜99%.

### Synthesis of Dendrimer-Drug Conjugate.

The solution of D-Hexyne and drug-azide in DMF (5 mL) was treated with copper sulfate pentahydrate (CuSO₄·5H₂O) and sodium ascorbate in water. The reaction mixture was stirred and heated for 10 h at 50° C. in a microwave synthesizer. On completion, the reaction mixture was dialyzed against DMF in 1 KDa cut-off cellulose dialysis tubing. To this solution, EDTA (50 μL, 0.5M) solution was added for copper removal by chelation. The DMF dialysis was followed by water dialysis overnight. The drug loading is calculated by proton integration where peaks corresponding to dendrimer and drug are compared.

### G2-Glucose Dendrimer (GD2)-Drug Conjugate

Generation 2 glucose dendrimer (GD2) consists of 24 glucose molecules (96 surface hydroxyl groups) used for conjugation. Glucose dendrimers primarily are made of glucose moieties comprised of the central core of Di-pentaerythritol and one or more branching units of monosaccharide glucose molecules. Unlike hydroxyl-terminated PAMAM dendrimer, glucose dendrimers primarily are taken up by injured neurons and found to specifically target hyperexcitable neurons in both culture and in vivo mouse model.

### Synthesis and Characterization of Glucose Dendrimer (GD2).

The GD synthesis was begun by reacting hexapropargylated core with AB4, β-D-glucose-PEG4-azide building via click reaction to obtain generation 1 glucose dendrimer (GD1). The OH groups on GD1 were propargylated to obtain GD1-Acetylene24, which was reacted with β-D-glucose-PEG4-azide to obtain generation 2 (GD2) with 24 glucose moieties, providing 96 surface hydroxyl groups. Further the Cy5 fluorescent tag was attached on GD2 by propargylation of ˜2-3 hydroxyl groups to bring alkyne containing GD2 dendrimer. The GD intermediates and final products were purified using dialysis and characterized using ¹H NMR. The physicochemical properties of GD2 dendrimer were also evaluated (Table 2).

TABLE 2
Physiochemical Properties of GD2

| Size | 4.4 | nm |
|---|---|---|
| Zeta Potential | −6.0 | |
| Number of Glucose | 24 | |
| HPLC purity | >99% | |
| Molecular weight | 12800 | Da |

### Synthesis and Characterization of GD2-Drug Conjugates

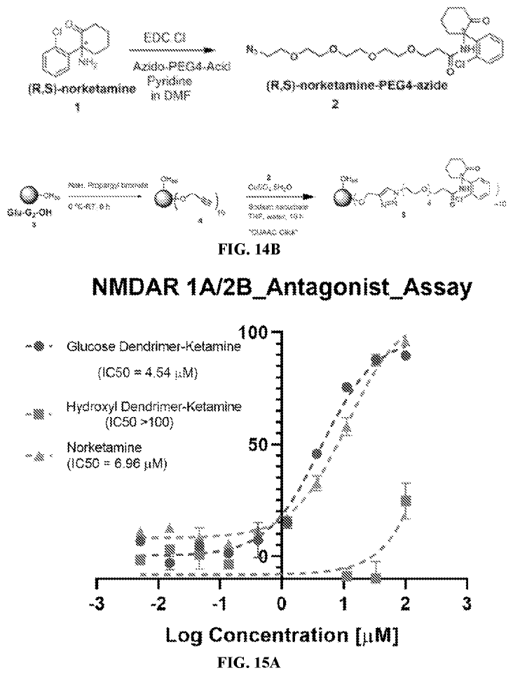

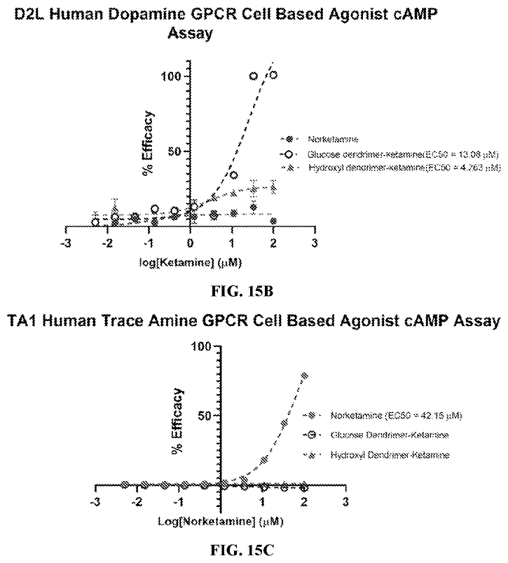

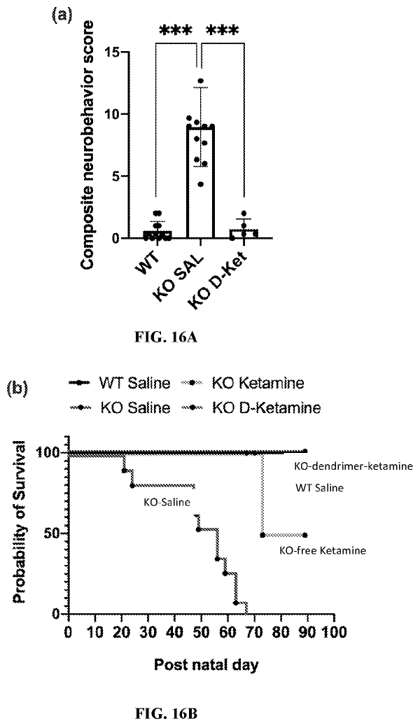

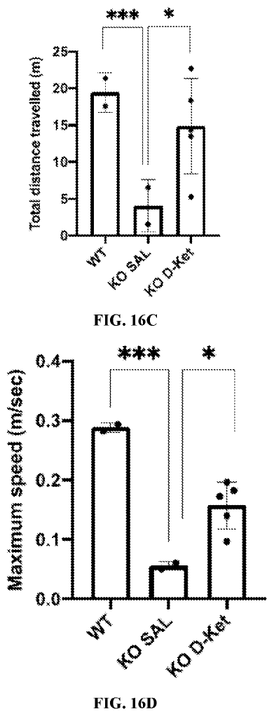

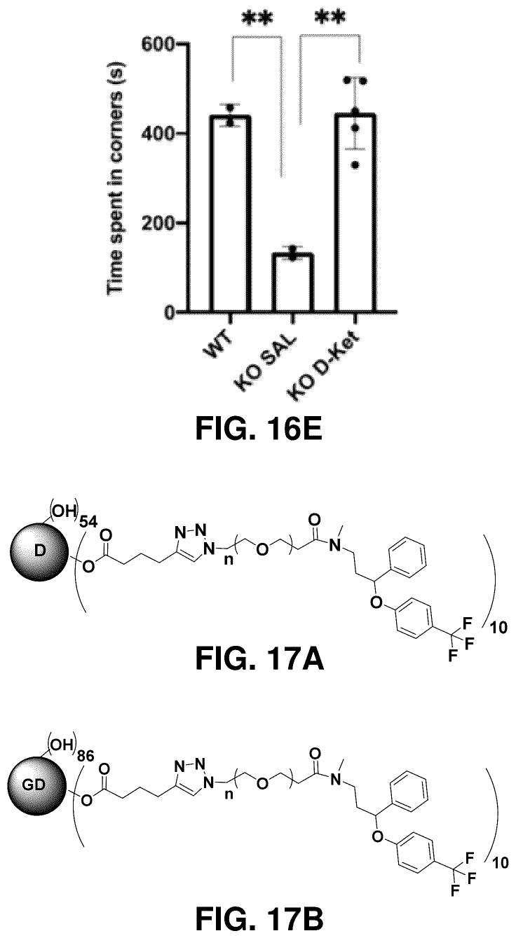

The Norketamine, tryptamine, venlafaxine and Hu308 drugs were conjugated to the GD2-Hexynoic acid dendrimer using click chemistry strategy. The linker attached drug moieties were conjugated to glucose dendrimer using Cu(I) catalyzed click (CuAAC) reaction in the presence of catalytic amount of CuSO₄·5H₂O and sodium ascorbate to obtain GD2-drug conjugate. The traces of copper were removed by dialyzing with ethylenediaminetetraacetic acid (EDTA). The final GD2-drug conjugates were characterized by NMR and HPLC. FIG. 14A is a schematic of the synthesis of a PAMAM dendrimer-norketamine conjugate. FIG. 14B is a schematic of the synthesis of a Glucose dendrimer-norketamine conjugate.

Characterization of the dendrimer-drug conjugates with venlafaxine and fluoxetine show that the conjugates are pure and are dramatically more soluble in water compared to free drugs, by >200-fold and >2900-fold respectively. This improved solubility will enabled them to be formulated in saline, without the use of relatively toxic formulations.

TABLE 3
Properties of Conjugated Compounds

| | | Conjugate | | | | |
|---|---|---|---|---|---|---|
| | Solubility | solubility | | | | |
| | of free | Based on | | | | |
| | drug mg/mL | drug mg/mL | | ¹H | | |
| Compound | (in water) | (in water) | | NMR | MALDI | DLS/nm |
| D-Norketamine | 0.178* | 30 | mg/mL | | | 3.9 ± 0.4 |
| D-Venlafaxine | PBS(pH 7.2): 10 | 50 | mg | | | 5.6 ± 0.1 |
| | mg/mL** | | | | | |
| D-Hu308 | Ethanol:PBS (Ph | | | | | 4.9 ± 0.2 |
| | 7.2)(1:2): 25 | | | | | |
| | mg/mL | | | | | |
| D-Tryptamine | 1.34* | 10 | mg/mL | | | 4.5 ± 0.1 |
| D-5HT | 4 mg/mL at 25° C. | Synthesis finished. | | | | |
| | | Continue with characterization | | | | |
| D-fluoxetine | 4 mg/mL | 38-40 | mg/mL | | | |
| D-Psilocyn | Psilocyn/Psilocybin linker chemistry is | | | | | |
| D-Psilocybin | established ready to start the conjugation | | | | | |
| GD-Norketamine | 0.178* | [symbol] 50 | mg/mL | | | |
| GD-Venlafaxine | PBS(pH 7.2): 10 | 50 | mg/mL | | | |

mg/mL**

[symbol]

indicates data missing or illegible when filed

TABLE 3
Drugs and Conjugates

| Active compound | Conjugatable analog |
|---|---|
| 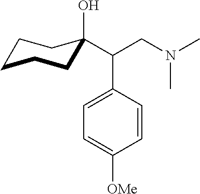 | 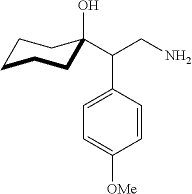 |
| Venlafaxine | 1-[2-amino-1-(4- |
| | methoxyphenyl)ethyl]cyclohexanol |
| 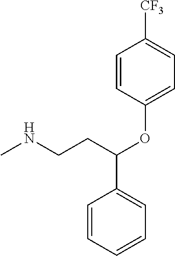 [SMILES: CNCCC(Oc1ccc(C(F)(F)F)cc1)c1ccccc1] | 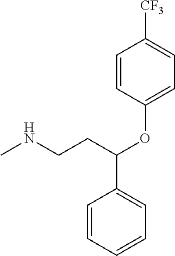 [SMILES: CNCCC(Oc1ccc(C(F)(F)F)cc1)c1ccccc1] |
| Fluoxetine | Fluoxetine |
| 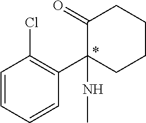 [SMILES: CNC1(c2ccccc2Cl)CCCCC1=O] | 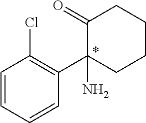 [SMILES: NC1(c2ccccc2Cl)CCCCC1=O] |
| (R,S)-ketamine | (R,S)-Norketamine |
| 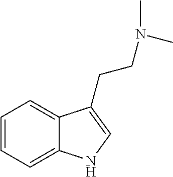 | 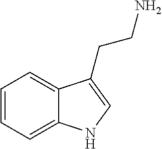 [SMILES: NCCc1c[nH]c2ccccc12] |
| N,N-DimethylTryptamine | Tryptamine |
| 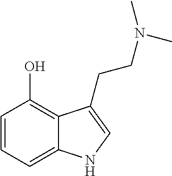 | 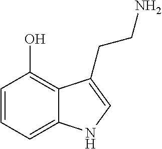 [SMILES: NCCc1c[nH]c2cccc(O)c12] |
| Psilocin | 4-Hydroxytryptamine |
| 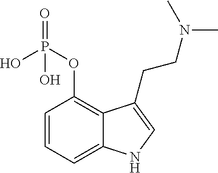 [SMILES: CN(C)CCc1c[nH]c2cccc(OP(=O)(O)O)c12] | 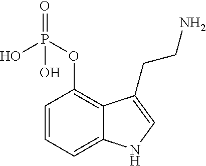 [SMILES: NCCc1c[nH]c2cccc(OP(=O)(O)O)c12] |
| Psilocybin | Norbeocystein |

TABLE 4
Physical properties of dendrimer-drug conjugates.

| | | Water | | |
|---|---|---|---|---|
| | | Solubility | | |
| | | dendrimer- | | |
| | Water | drug | | |
| Dendrimer | Solubility | conjugates | | |
| drug | of Free | (based on | Number of | |
| conjugate | drug* | free drug) | attachments | DLS/ nm |
| Hydroxyl | 0.23 mg/mL | 50 mg/mL | ~10 | 5.6 ± 0.1 |
| dendrimer- | | | | |
| Venlafaxine | | | | |
| conjugate | | | | |
| Hydroxyl | 0.0017 mg/mL | 5 mg/mL | ~5 | 4.6 ± 0.2 |
| dendrimer- | | | | |
| Fluoxetine | | | | |
| conjugate | | | | |
| Glucose | 0.23 mg/mL | 50 mg/mL | ~10 | |
| dendrimer- | | | | |
| Venlafaxine | | | | |
| conjugate | | | | |

*https://go.drugbank.com/metabolites/DBMET00189

### Example 4: Binding Assays of Dendrimer-Ketamine Conjugates

### Materials and Methods

### Human Serotonin 5-HT2A Receptor (Agonist Radioligand):

Purpose: Evaluation of the affinity of compounds for the human 5-HT2A receptor in transfected HEK-293 cells determined in a radioligand binding assay.

Experimental protocol: Cell membrane homogenates (30 μg protein) are incubated for 60 min at 22° C. with 0.1 nM [125I]DOI in the absence or presence of the test compound in a buffer containing 50 mM Tris-HCl (pH 7.4), 5 mM MgCl2, 10 μM pargyline and 0.1% ascorbic acid.

Nonspecific binding is determined in the presence of 1 μM DOI. Following incubation, the samples are filtered rapidly under vacuum through glass fiber filters (GF/B, Packard) presoaked with 0.3% PEI and rinsed several times with ice-cold 50 mM Tris-HCl using a 96-sample cell harvester (Unifilter, Packard). The filters are dried then counted for radioactivity in a scintillation counter (Topcount, Packard) using a scintillation cocktail (Microscint 0, Packard).

The results are expressed as a percent inhibition of the control radioligand specific binding.

The standard reference compound is DOI, which is tested in each experiment at several concentrations to obtain a competition curve from which its IC50 is calculated.

See Bryant, et al. (1996), a novel class of 5-HT2A receptor antagonist: aryl aminoguanidines, Life Sci., 15: 1259.

Delta (DOP) Human Opioid GPCR Cell Based Antagonist cAMP Assay:

Purpose: Evaluation of the potency (IC50) and efficacy (Max response) of compounds for the human delta (DOP) receptor in stably transfected CHO-K1 cells. Assay principle is cAMP cell-based assay.

Experimental protocol: Cells were seeded in a total volume of 20 μL into white walled, 384-well microplates and incubated at 37° C. overnight. Prior to testing cell plating media was exchanged with 10 μL of Assay buffer (HBSS+10 mM HEPES)

Briefly, intermediate dilution of sample stocks was performed to generate 4× sample in assay buffer. 5 μL of 4× sample was added to cells and incubated at 37° C. for 30 minutes. 5 μL of 4×EC80 in 4× forskolin reagent was added and cells incubated for 37° C. for 30 minutes. Final assay vehicle concentration was 1%. The results are expressed as a percent inhibition of the control ligand.

### GENERAL INFORMATION

Assay volume and format: 10 μl in 384-well plate
Compound addition: 5 ul of 4× compound.
Maximum tolerable DMSO concentration: Customer compounds (when in DMSO) are diluted as [100×] solution in solvent. 1%
Assay Temperature: 37° C.
Incubation time: 30 mins
Forskolin Concentration: 20 μM
   TA1 Human Trace Amine GPCR Cell Based Agonist cAMP Assay

Purpose: Evaluation of the potency (EC50) and efficacy (Max response) of compounds for the human TAI receptor in stably transfected CHO-K1 cells determined in a GPCR cell based cAMP assay.

Experimental protocol: Cells were seeded in a total volume of 20 μL into white walled, 384-well microplates and incubated at 37° C. overnight prior to testing.

Prior to testing cell plating media was exchanged with 15 μL of Assay buffer (HBSS+10 mM HEPES). Briefly, intermediate dilution of sample stocks was performed to generate 4× sample in assay buffer. 5 μL of 4× was added to cells and incubated at 37° C. for 30 minutes. Final assay vehicle concentration was 1%.

The results are expressed as a percent efficacy relative to the maximum response of the control ligand.

### General Information

Assay volume and format: 20 μl in 384-well plate
Compound addition: 5 ul of 4× compound. Customer compounds (when in DMSO) are diluted as [100×] solution in solvent.
Maximum tolerable DMSO 1%
concentration:
Assay Temperature: 37° C.
Incubation time: 30 mins

### Results

TABLE 5
Results of Binding Assays

| | NMDAR | Opiate μ | Opiate k | Sigma 2 |
|---|---|---|---|---|
| NorKetamine | 0.33 μM | Not | 667 μM | 246 μM |
| | | active | | |
| Glucose Dendrimer- | Not | 91 μM | 143 μM | 147 μM |
| Ketamine | active | | | |
| Hydroxyl-terminated | 1.7 μM | 64 μM | 98 μM | 68 μM |
| PAMAM Dendrimer- | | | | |
| Ketamine | | | | |

| | | | | NMDAR |
|---|---|---|---|---|
| | | | | 1A/2B |
| | | Opiate | | Ion |
| | DRD2L | delta1R | 5HT1AR | channel |
| | Agonist | (antagonist) | agonist | blocker |
| NorKetamine | Not | Not | | 6.9 μM |
| | active | active | | |
| Glucose Dendrimer- | 13.07 μM | Not | 23.13 μM | 4.6 μM |
| Ketamine | | active | | |
| Hydroxyl-terminated | 4.3 μM | Not | Not | Not |
| PAMAM Dendrimer- | | active | active | active |
| Ketamine | | | | |

FIG. 15A is a graph of an NMDAR 1A/2B antagonist assay for glucose dendrimer-ketamine (IC50=4.54 μM), hydroxyl dendrimer-ketamine (IC50>100), and norketamine (IC50=6.96 μM). FIG. 15B is the % binding efficacy of the log concentration of compound in micromolar in a D2L human dopamine GPCR cell based agonist cAMP assay. Norketamine (solid circle), glucose dendrimer-ketamine EC50=13.08 micromolar (open circle), and hydroxyl dendrimer-ketamine EC50=4.263 micromolar (triangle). FIG. 15C is the % efficacy of the log concentration of ketamine in micromolar in the TA1 human trace amine GPCR cell based agonist cAMP assay. Norketamine (solid circle), glucose dendrimer-ketamine EC50=13.08 micromolar (open circle), and hydroxyl dendrimer-ketamine EC50=4.263 micromolar (triangle).

Significant efficacy data was obtained with a clinically important drug that is derivative of ketamine (norketamine). Norketamine has been undergoing trials for depression and addiction. The hydroxyl dendrimer-drug target these receptors in microglia/macrophages, whereas glucose dendrimer-drug target these receptors both in neurons and microglia.

The conjugates can be active with or without releasing the drug. Binding affinities measure the activity of the intact conjugates. Typically, conjugates of psychedelic drugs are prepared in the non-releasing form (e.g. tryptamine, psilocin, psilocybin, ketamine), and are intended to be active in the intact form. This enables the intact conjugates to be released through the kidney with no toxicity from drug release. Alternatively, they can be designed to release through analogs.

Both GD-ketamine and HD-ketamine show effects that are unique and should be beneficial for neuropsychiatric conditions. Both dendrimer-ketamine conjugates show greater binding than nor-ketamine for opiate u, opiate k and sigma 2 receptors, indicating that dendrimer conjugation provides positive benefits to binding these receptors. Norketamine does not bind Opioid μ receptors (Ki for GD-ket is 53 μM and 40 μM for HD-ket). Ki for opioid k is 381 μM for norketamine while it is about 4 fold better for GD-ket (Ki=82 μM) and ˜7 fold better for HD-Ket (Ki=556 μm). The anti-depressant effects of ketamine are thought to be mediated through the opioid k receptors and dendrimer binding increases the affinity for those receptors.

Improved binding upon dendrimer conjugation is also seen with sigma 2 receptors. The greater ability of HD ketamine to bind Sigma 2 receptors indicates greater neuroprotective effects. The mechanism of neuroprotection may be related to increased NGF and BDNF production. Sigma 2 receptor activation can also be beneficial in neuropsychiatric conditions such as schizophrenia and psychosis. Decreased anxiety and greater anti-depressant effects can be seen with sigma 2 receptor targeting. Ketamine binds sigma2 receptors at mM concentrations. (Pergolizi, 2023; Bonaventura, 2021)

The ability to bind dopamine 2 receptors is seen only with the dendrimer-conjugated ketamine. Free norketamine does not appear to bind Dopamine 2 receptors. Similarly, ketamine has also not shown any affinity for dopamine 2 receptors. The dopaminergic effects of ketamine are believed to be indirect effects. However, it was shown that glucose dendrimer-ketamine has an RC50 of 13.07 μM at D2 receptors while hydroxyl dendrimer-ketamine is more effective with an RC50 of 4.3 μM. The anti-depressant and neuroprotective effects of ketamine are mostly mediated through these and the dopamine receptors. Ketamine, norketamine and metabolites of ketamine do not appear to have a direct effect on dopamine receptors are do not bind dopamine receptors. The increased activity seen at the D2 receptors on the functional assay indicates that dendrimer-ketamine will be more effective as an anti-depressant.

GD-ketamine and HD-ketamine show different features. Binding with hydroxyl dendrimer (HD) as compared to glucose dendrimer (GD) appears to change the function of ketamine, which is highly expected. HD-ketamine does not demonstrate NMDAR 1A/2B ion channel blockage while GD ketamine does demonstrate that the type of dendrimer that is bound to ketamine is critical for differences in function between them. GD-ketmaine also shows agonist activity against 5HT1A receptors which is not seen with HD-ketamine.

These results indicate that dendrimer conjugation produces unexpected benefits to free drugs on receptor binding and is dependent on the dendrimer structure. The microglial targeting of hydroxyl dendrimer and the additional neuronal targeting of the glucose dendrimers bring unexpected results with clinical significance.

The conjugates are targeted to specific receptors on whatever cells they may be on. This includes neurons, microglia, macrophages and other cells. These receptors may be anywhere in the brain. Examples include serotonin receptors such as 5HTIA, 5HT2A, NMDA etc. Serotonin receptors can also be found in microglia. The compounds bind to and act on these receptors. The ‘intrinsic cellular targeting’ of these dendrimers (hydroxyl dendrimer-microglia/macrophages, and glucose dendrimers- to neurons) are somewhat secondary to the action on the specific receptors since the compositions target specific receptors. Important receptors include the serotonin receptors [5HT1A, 5HT2A (agonism, antagonism, reverse or inverse agonism)] and NMDA receptors. In many cases, binding to specific receptors and not binding to other receptors using dendrimers can enhance binding efficacy and reduce side effects of these drugs.

When combined with the cellular targeting capability of glucose dendrimers, such as to injured neurons (primary), microglia/macrophages (secondary), or hydroxyl dendrimers (microglia/macrophages), improvement in water solubility of >5-200-fold is shown with dendrimer conjugation, there are clear benefits to these conjugates: modifiable binding, increased selectivity of targeting, increased ease of formulation and delivery, and reduced side effects.

Using Tryptamine as an example, it was shown that binding affinities of drugs conjugated with these dendrimers have unexpected properties.

Both hydroxyl and GD dendrimers have OH surface groups. When tryptamine was conjugated to these dendrimers with the same linking chemistry, very different affinities were seen to serotonin and other receptors. The conjugates showed less affinity than free drug in cell-based binding assays (indicative of in vivo efficacies). The lower affinity may enable less tight binding and may enable us to modulate the undesirably strong effects of the drugs on this receptor. Second, the hydroxyl dendrimer conjugate was not active, but the glucose dendrimer conjugate was active. This was not expected and may be due to the differential internal structure of the glucose and hydroxyl dendrimers. The drug may fold into the hydrophobic core of the hydroxyl dendrimer but may open outwards in the hydrophilic interior of the glucose dendrimer.

### Example 5: Treatment of Rett Syndrome Animal Model with Ketamine and Ketamine-Dendrimer Conjugates

Rett syndrome (RTT) is an inherited neurodevelopmental disorder of females that occurs once in 10,000-15,000 births. Affected females develop normally for 6-18 months, but then lose voluntary movements, including speech and hand skills. Most RTT patients are heterozygous for mutations in the X-linked gene MECP2, encoding a protein that binds to methylated sites in genomic DNA and facilitates gene silencing. The symptoms, progression, and severity of Rett syndrome can vary dramatically from one person to another. A wide range of disability can potentially be associated with Rett syndrome. Symptoms generally appear in stages. RTT is typically characterized by a period of normal development after birth, followed by regression in speech and hand movements, gait abnormalities, erratic hand movements, and deceleration of head growth. Other diagnostic criteria for RTT include irregular breathing, gastrointestinal and musculoskeletal disorders, seizures, poor sleep, reduced response to physical pain, and behavioral issues.

Ketamine is a well established anesthetic drug that results in ‘dissociateive anesthesia’ and exerts both central and peripheral effects including hypnosis, analgesia and sympathomimetic effects leading to hypertension and tachycardia. The main mechanism is felt to be due to its role as an antagonist at the N-methyl-d-aspartate (NMDA) receptor. This can lead to rapid action and response seen with treatment for treatment resistant depression, MDD and suicidal ideations, unlike SSRIs and SNRIs that only show delayed effects in controlling depression. Ketamine is also potent as a therapy in chronic pain, again due to its effects on NMDAR inhibition. Increased dopamine release and its role on However, ketamine also exerts neuroprotective effects by non-NMDAR mediated mechanisms such as increasing BDNF and mTOR. Ketamine also exerts effects on opioid receptors, can increase dopamine. These effects are beneficial for chronic pain, treatment resistant depression, MDD, and treatment resistant epilepsy/seizures.

However, ketamine has several short term and long term side effects. Ketamine can lead to respiratory depression at high doses, can lead to systemic side effects such as increased heart rate, hypertension, hyperthermia, loos of coordination, dizziness, nausea, vomiting, disturbing laternations in sensory perceptions and high incidence of auditory and visual hallucinations. More than half the patients who are treated with ketamine develop and emergence phenomenon when ketamine wears off that is characterized by euphoria, vivid dreams, hallucinations, illusions, distortions in body images and objects and delirium that can be extremely disturbing and may lead to self injury. Some of these symptoms correlate with symptoms of schizophrenia. Long term use of ketamine can lead to memory impairments and decline in executive functioning. Ketamine also leads to tolerance and is addictive leading to withdrawal and dependence. Due to these significant side effects, ketamine can only be administered in a controlled setting.

Ketamine has been shown to be effective in a mouse model of Rett syndrome at a large dose of 8 mg/kg delivered IP every day for 40 days (total dose of 320 mg/kg) and improved survival by 50% at 80 days post natal (Patrizi, et al. 2016)

### Materials and Methods

Dendrimer conjugates were prepared as described above.

As reported by Guy, et al. a mouse Mecp2-null mutation causes neurological symptoms that mimic Rett syndrome. Nat Genet. 2001 March; 27 (3): 322-6. doi: 10.1038/85899. PMID: 11242117 Both Mecp2-null mice and mice in which Mecp2 was deleted in brain show severe neurological symptoms at approximately six weeks of age.

Patrizi A, et al. Chronic Administration of the N-Methyl-D-Aspartate Receptor Antagonist Ketamine Improves Rett Syndrome Phenotype. Biol Psychiatry. 2016 May 1; 79 (9): 755-764. doi: 10.1016/j.biopsych.2015.08.018. Epub 2015 Aug. 24. PMID: 26410354; PMCID: PMC7410367, reported on a systematic, randomized preclinical trial of chronic administration of low-dose (8 mg/kg, intraperitoneal) ketamine, an NMDAR antagonist, starting either early in development or at the onset of RTT phenotype in Mecp2-null mice. Mice were treated from day 15 to day 55 or day 30 to day 55 at 8 mg ketamine/kg/day ip (total dose of 320 mg/kg for 40 days or 200 mg/kg for 25 days). Treatment from day 30 was not effective while treatment from day 15 showed some efficacy at 320 mg/kg total dose.

### Experimental Paradigm for MECP2 Knock Out (“KO”) Mice:

Treatment from 28 days of age (4 weeks of age, symptomatic). Untreated animals die by around 55-60 days.

Mice were treated with ketamine or ketamine conjugated to dendrimer. Controls were wild type treatment with saline, knock out treated with saline.

Animals were administered treatment twice weekly ip in a 2.5 mg/kg/dose. Total dose at time of assessment (60 days of age) is about 22.5 mg/kg.

Dendrimer ketamine was tested in the Rett syndrome mouse model IP at a dose of 2.5 mg/kg (ketamine) biweekly for 8 weeks (total dose of ketamine 40 mg/kg).

4 weeks old Mecp2 Kos (knock out) were randomly divided and treated biweekly for 8 weeks with saline or 2.5 mg/Kg ip of Ketamine or D-Ketamine (WT-saline, KO-saline, KO-Ketamine, KO D-Ketamine groups). The weekly neurobehavior was evaluated by recording their composite neurobehavior score (NBS) based on a scale that includes assessments of mobility, gait, paw clasping, tremors, and respiration on a scale of 0-3 each; the higher the score, the worse the phenotype. D-ketamine treated group showed slowed the progression of disease phenotype with better neurobehavior scores post treatment, whereas the untreated KOs did not.

| | |
|---|---|
| Score | Description |

Mobility

| | |
|---|---|
| 0 | normal |
| 1 | normal speed but not climbing (slower) |
| 2 | Slow/only moves when prodded (little |
| | movement) |
| 3 | No spontaneous movement even when prodded |

Gait

| | |
|---|---|
| 0 | normal speed but not climbing (slower) |
| 1 | waddle |
| 2 | waddle-fee pointed outwad/walking on toes |
| 3 | severe waddle, shuffling; unable to |
| | take clean steps |

Paw Clench

| | |
|---|---|
| 0 | no clench |
| 1 | little/infrequent (1 instance) |
| 2 | intermittent |
| 3 | constant |

Respiration

| | |
|---|---|
| 0 | normal |
| 1 | slow or fast hiccups |
| 2 | hiccups and irregular rate |
| 3 | apneas |

Open Field Test: The long term behavioral changes in Saline (WT), Ketamine and D-Ketamine groups versus KO-saline at 8.5th week of treatment. The motor function was assessed by recording the mice in an open field arena (10.5″×19″×8″). Mice were recorded in the same room where they were housed to avoid the stress and variability in the test procedure. Each mouse was placed in a clean open field arena and was allowed to explore for 10 minutes, and activity was recorded. Animals were placed in same way in the open field 5 cm away facing the longer wall.

### Results

Animals were assessed for survival, neurobehavior score and activity (total distance traveled, speed, and time spent in corners).

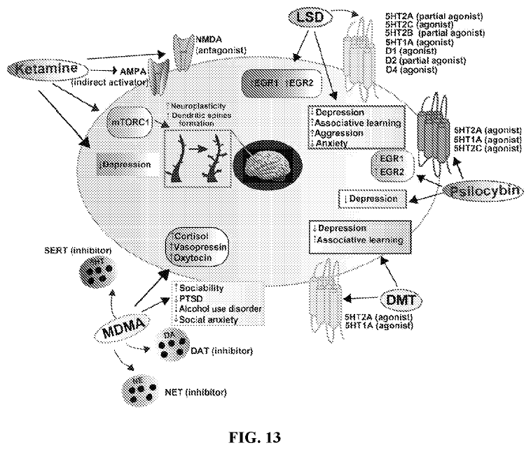

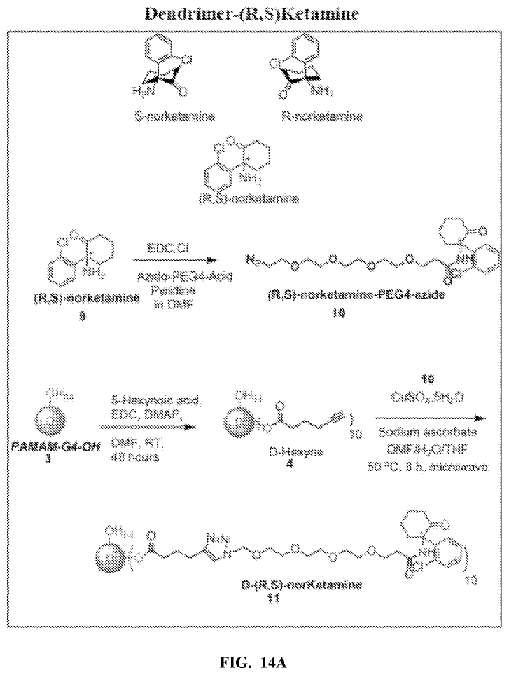

FIGS. 13A and 13B are graphs of wild type, knock out saline (controls) versus knockout mice treated with dendrimer-ketamine conjuate composite neurobehavior score of (FIG. 13A) and probability of survival over post natal day (FIG. 13B). FIG. 13C is a graph of the composite neurobehavioral score; FIG. 13D is a graph of the distance traveled (m).

Dendrimer conjugated with ketamine leads to increased efficacy with increased binding to NMDAR without the associated side effects. The dose used is significantly lower than free ketamine that will reduce the side effects. This is tested in a mouse model of Rett syndrome as a proof of concept since Rett Syndrome is a disease that has increased glutamate production and increased NMDAR expression/activation.

Significant improvement in survival with 100% survival was seen up to 90 days of age with D-ketamine compared to untreated animals and animals treated with free ketamine. Previous published data by others in a similar model shows only 50% survival after treatment with 320 mg/kg of ketamine (8 times higher dose).

Significant improvement in motor function is seen with D-ketamine, with behavior similar to that of normal healthy controls.

Significant differences were seen in the total distance travelled by the WT versus KO mice in the open field for 10 minutes of duration. Significant improvement was also observed in motor function represented as the increment in the total distance travelled by the D-Ketamine treated KOs in open field test. Significant improvement were observed in maximum speed and time spent in corners by D-Ketamine in comparison to the KO Saline group.

Videos show dramatic improvement in phenotype bringing them close to healthy mice.

1. A composition comprising dendrimers, preferably hydroxyl-terminated dendrimers, sugar-terminated dendrimers, and/or sugar-based dendrimers, covalently conjugated to at least one antidepressant and/or antipsychotic compound, wherein the psychedelic and hallucinogenic agents are not ketamine, methoxetamine, a derivative of ketamine, a cannabinoid, ibogaine, or a derivative of a cannabinoid or ibogaine.

2. The compositions of claim 1, where the covalent conjugation is a covalent link between a modified or unmodified surface group or interior groups of the dendrimer, including an amide, ester, disulfide, ether, or phosphate linkage.

3. The composition of claim 1, wherein the covalent link between the dendrimer and antidepressant and/or antipsychotic compounds is cleaved following administration.

4. The composition of claim 1 where the dendrimer is a glucose dendrimer or polyamidoamine (PAMAM) dendrimer of generation 1-Generation 10 (such as generation 1, generation 2, generation 3, generation 4, generation 5, generation 6, generation 7, generation 8, generation 9, or generation 10), preferably with between greater than 40 and 100% of the surface groups being hydroxylated or conjugated to monosaccharides.

5. The composition of claim 1 wherein the dendrimer is a glucose dendrimer, preferably generation 1, generation 2, or generation 3 glucose dendrimers.

6. The composition of claim 1 where the dendrimer is a generation 2-generation 7 PAMAM dendrimer, modified by a sugar moiety, wherein the sugar group is selected from the group consisting of glucose, galactose, mannose, and fructose.

7. The composition of claim 1 where the dendrimer is a dendritic polymer, optionally a hyperbranched polymer.

8. The composition of claim 1 where the dendrimer is a PAMAM dendrimer (such as a hydroxyl-terminated PAMAM dendrimer) of generation 2-Generation 10, preferably with greater than 40% of the functional groups in the form of hydroxyl (OH) or monosaccharide groups.

9. The compositions of claim 1 where the dendrimer is a glucose dendrimer, made of glucose and optionally ethylene glycol building blocks, with greater than 10 surface glucose moieties.

10. The compositions of claim 1 where the dendrimer is a galactose dendrimer, made of galactose and optionally ethylene glycol building blocks, with greater than 10 surface sugar moieties.

11. The compositions of claim 1 where the dendrimer is a sugar dendrimer, made of sugar and optionally ethylene glycol building blocks, with greater than 10 surface sugar moieties, optionally where the sugar building blocks are mannose or galactose.

12. The composition of claim 1 where the compound is a selective serotonin reuptake inhibitors (SSRI), serotonin-norepinephrine reuptake inhibitors (SNRI), antipsychotics, atypical antipsychotics, tricyclic antidepressant (TCA), monoamine oxidase inhibitors (MAOI), benzodiazepines, beta-blockers (BB), and anticonvulsants.

13. The composition of claim 12 where the antidepressant is a serotonin receptor antagonist, preferably selected from the group consisting of citalopram, escitalopram, fluoxetine, paroxetine, paxparoxetine, vilazodone, sertraline, fluvoxamine, an analogue, or a derivative thereof.

14. The composition of claim 12 where the antidepressant is a serotonin and norepinephrine receptor antagonist, preferably selected from the group consisting of imipramine, nortriptyline, amitriptyline, doxepin, desipramine, duloxetine, venlafaxine, esvenlafaxine, levomilnacipran, an analogue, or a derivative thereof.

15. The composition of claim 12 where the antidepressant is a MAO-A and/or MAO-B receptor antagonist, wherein the antidepressant is selected from the group consisting of tranylcypromine, phenelzine, socarboxazid, selegiline.

16. The composition of claim 12 where the antidepressant alters neuronal excitability, preferably wherein the antidepressant is ganaxolone, minaxolone, zuranolone, an analogue, or a derivative thereof.

17. The composition of claim 12 where the antidepressant is a norepinephrine and dopamine receptor antagonist, preferably where the antidepressant is bupropion and/or mirtazapine, an analogue, or a derivative thereof.

18. The composition of claim 12 where the antipsychotic compound is a typical antipsychotic and/or an atypical antipsychotic, preferably where the antipsychotic is quetiapine, haloperidol, risperidone, olanzapine, clozapine, an analogue, or a derivative thereof.

19. The composition of claim 12 wherein the compound is selected from the group consisting of methylphenidate, amphetamine, atomoxetine, clonidine, guanfacine, viloxazine, an analogue, or a derivative thereof.

20. The composition of claim 1 wherein the dendrimer-antidepressant and/or antipsychotic compositions have at least a 10-fold increase in drug solubility in comparison to the free antidepressant and/or antipsychotic or wherein the dendrimer-antidepressant and/or antipsychotic compositions reduce the onset of action in comparison to the free antidepressant and/or antipsychotic.

21. The composition of claim 1, wherein the dendrimer-antidepressant and/or antipsychotic compositions reduce the time to onset of drug action by 50% or better.

22. The composition of claim 1 wherein the dendrimer-antidepressant and/or antipsychotic compositions reduce the side effects of free drugs.

23. The composition of claim 1 where the dendrimer-antidepressant and/or antipsychotic conjugates further comprises pharmaceutically acceptable excipients.

24. The composition of claim 1, comprising higher generation dendrimer, preferably generation 3, 4, 5, or 6 PAMAM dendrimer (such as hydroxyl-terminated PAMAM dendrimer), generation 1, 2, 3, or higher glucose dendrimer, or functionalized with PEG, wherein the conjugate is confined to the peripheral circulation.

25. The composition of claim 1, wherein the at least one antidepressant and/or antipsychotic compound is conjugated to the dendrimers via a spacer comprising a hydrocarbon (such as an alkylene), a diethylene glycol moiety, oligoethylene glycol chain, a triazole moiety, or a combination thereof, preferably wherein the spacer comprises a triazole moiety.

26. A method of administering the dendrimer conjugates of claim 1 to an individual in need thereof, preferably administered via a route selected from the group consisting of mucosal administration, enteral administration or injection, preferably intranasal, intravenous, oral, sublingual, subcutaneous, inhalable, transdermal, intraperitoneal, or intrathecal.

27. The method of claim 26 comprising preventing or treating depression such as major depressive disorder, treatment-resistant depression, and post-partum depression, post-traumatic stress disorder, panic disorder, social anxiety disorder, anorexia nervosa, suicidal ideation, obsessive-compulsive disorder, ADHD or symptoms thereof, premenstrual dysphoric disorder, anorexia, substance abuse disorders, epilepsy, bi-polar disorder, autism spectrum disorders, attention-deficit hyperactivity disorder, schizophrenia, cluster headaches, migraines, seizures, fibromyalgia, narcolepsy, obesity, Alzheimer's disease, Tourette's syndrome, pain such as neuropathic pain and chronic pain, phobias, and cardiovascular diseases,
where the compositions contain the dendrimers conjugated with or formulated with therapeutic agents such as Selective Serotonin Reuptake Inhibitors (SSRIs), Serotonin and Norepinephrine Reuptake Inhibitors (SNRIs), Norepinephrine-Dopamine Reuptake Inhibitors (NDRIs), Tricyclic Antidepressants (TCAs), Monoamine Oxidase Inhibitors (MAOIs), Benzodiazepines, GABA modulators such as neurosteroids, antipsychotics, atypical antipsychotics, or analogues thereof, where the composition is delivered via oral, subcutaneous, transdermal, intranasal, intramuscular, intravenous, or intrathecal routes.

28. The method of claim 27 comprising treating neuroinflammatory mental health disorders by modulating activation of receptors on microglial cells by administering the dendrimer-agent conjugates.

29. The method of claim 27 comprising treating mental health disorders by modulating activation of receptors on neurons by administering the dendrimer-agent conjugates.
